(19) United States
(12) Patent Application Publication
(10) Pub. No.: US 2004/0224939 A1
(43) Pub. Date: Nov. 11, 2004

(54) Antibacterial agents

(76) Inventors: Louis S. Chupak, Old Saybrook, CT (US); Frederick Earl Boyer JR., Canton Township, MI (US); Susan Hagen, Canton, MI (US); Takushi Kaneko, Guilford, CT (US); Manjinder Lall, Ann Arbor, MI (US); Martin Pettersson, Austin, TX (US); Joysula Venkata Nagendra Vara Prasad, Ann Arbor, MI (US)

Correspondence Address: WARNER-LAMBERT COMPANY, 2800 PLYMOUTH RD, ANN ARBOR, MI, 48105, US

(21) Appl. No.: 10/773,588

(22) Filed: Feb. 5, 2004

Related U.S. Application Data
(60) Provisional application No. 60/445,957, filed on Feb. 7, 2003.

(51) Int. Cl. A61K 31/55

(52) U.S. Cl. 514/215; 540/593

(57) ABSTRACT

Compounds of formula I and methods for their preparation are disclosed. Further disclosed are methods of making biologically active compounds of formula I as well as pharmaceutically acceptable compositions comprising compounds of formula I. Compounds of formula I as disclosed herein can be used in a variety of applications including use as antibacterial agents.

This Regular Application claims benefit of U.S. Provisional Appliation No. 60/445,957, filed Feb. 7, 2003.

### FIELD OF THE INVENTION

The invention relates to compounds bearing an oxazolidinone core structure which exhibit antibacterial activity, methods for their preparation, as well as pharmaceutically acceptable compositions comprising such compounds.

### BACKGROUND OF THE INVENTION

The oxazolidinones form a novel class of antibacterial agents with potent activity against a number of human and veterinary pathogens, including gram-positive aerobic bacteria such as multiply-resistant staphylococci and streptococci, anaerobic organisms such as bacteroides and clostridia species, and acid-fast organisms such as Mycobacterium tuberculosis and Mycobacterium. However, oxazolidinones generally do not demonstrate useful activity levels against aerobic gram-negative organisms. As a result, the use of oxazolidinones is limited to infections due to gram-positive bacteria. Accordingly, there is a need for oxazolidinones that have broader antibacterial activity, including activity against gram-negative as well as gram positive organisms.

### SUMMARY OF THE INVENTION

These and other needs are met by the present invention, which is directed to a compound of formula I:

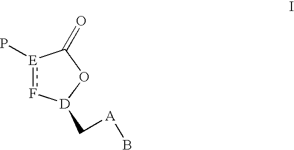

or a pharmaceutically acceptable salt thereof, wherein:

A is O,

NH, or

S;

B is

C(═O)R₁,

C(═S)R₁,

heterocylco,

heteroaryl,

C(═O)-heterocyclo,

C(═N)—CN, or

C(═O)-heteteroaryl;

either D is N, E is C, and F is CH when “------” is a bond, or D is CH, E is N, and F is CH₂ when “------” is absent;

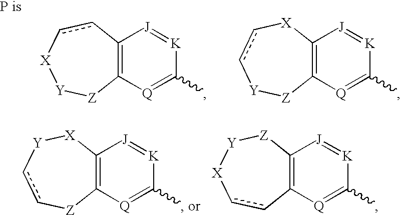

wherein “ [symbol] ” indicates the point of attachment;

J, K, Q independently are CR₂ or N, with the proviso that when any one of J, K, or Q is N, then the other two are CR₂;

“------” is absent; or is a bond; and

X, Y, Z independently are C═C—R₅,

O═C,

CH₂,

CHR₃,

CHR₄,

CR₃R₄,

NR₅,

N(C═O)R₅,

N(C═O)OR₅,

NSO₂R₅,

NSO₂NR₅,

O,

S,

SO, or

SO₂;

R₁ is H,

(C₁-C₈)alkyl,

(C₃-C₆)cycloalkyl,

O—(C₁-C₄)alkyl,

O—(C₃-C₆)cycloalkyl,

S—(C₁-C₄) alkyl,

S—(C₃-C₆)cycloalkyl,

NH₂,

NH(C₁-C₄)alkyl,

N((C₁-C₄)alkyl)₂, or

NH—(C₃-C₆)cycloalkyl;

R₂ is H,

halo,

(C₁-C₈)alkyl,

(C₃-C₆)cycloalkyl,

O—(C₁-C₄)alkyl,

O—(C₃-C₆)cycloalkyl,

S—(C₁-C₄) alkyl,

S—(C₃-C₆)cycloalkyl,

NH₂,

NH(C₁-C₄)alkyl,

N((C₁-C₄)alkyl)₂, or

NH—(C₃-C₆)cycloalkyl;

R₃ and R₄ independently are halo,

(C₁-C₈)alkyl,

(C₃-C₆)cycloalkyl,

O—(C₁-C₄)alkyl,

O—(C₃-C₆)cycloalkyl,

S—(C₁-C₄) alkyl,

S—(C₃-C₆)cycloalkyl,

NH₂,

NH(C₁-C₄)alkyl,

N((C₁-C₄)alkyl)₂,

NH—(C₃-C₆)cycloalkyl;

aryl,

(CH₂)ₙ-aryl,

heterocyclo,

(CH₂)ₙ-heterocyclo,

heteroaryl, or

(CH₂)ₙ-heteroaryl,

wherein n is 0, 1, 2, or 3;

R₅ is H,

(C₁-C₈)alkyl,

(C₃-C₆)cycloalkyl,

aryl,

(CH₂)ₙ-aryl,

heterocyclo,

(CH₂)ₙ-heterocyclo,

heteroaryl, or

(CH₂)ₙ-heteroaryl,

wherein n is as defined above.

What is also provided is a compound of formula II

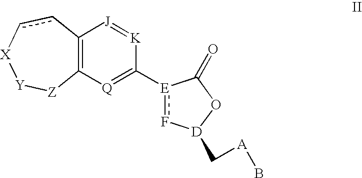

or a pharmaceutically acceptable salt thereof, wherein:

A is O,

NH, or

S;

B is

C(═O)R₁,

C(═S)R₁,

heterocylco,

heteroaryl,

C(═O)-heterocyclo,

C(═N)—CN, or

C(═O)-heteteroaryl;

either D is N, E is C, and F is CH when “------” is a bond, or D is CH, E is N, and F is CH₂ when “------” is absent;

J, K, Q independently are CR₂ or N, with the proviso that when any one of J, K, or Q is N, then the other two are CR₂;

“------” is absent; or is a bond; and

X, Y, Z independently are C═C—R₅,

O═C,

CH₂,

CHR₃,

CHR₄,

CR₃R₄,

NR₅,

N(C═O)R₅,

N(C═O)OR₅,

NSO₂R₅,

NSO₂NR₅,

O,

S,

SO, or

SO₂;

R₁ is H,

(C₁-C₈)alkyl,

(C₃-C₆)cycloalkyl,

O—(C₁-C₄)alkyl,

O—(C₃-C₆)cycloalkyl,

S—(C₁-C₄) alkyl,

S—(C₃-C₆)cycloalkyl,

NH₂,

NH(C₁-C₄)alkyl,

N((C₁-C₄)alkyl)₂, or

NH—(C₃-C₆)cycloalkyl,

R₂ is H,

halo,

(C₁-C₈)alkyl,

(C₃-C₆)cycloalkyl,

O—(C₁-C₄)alkyl,

O—(C₃-C₆)cycloalkyl,

S—(C₁-C₄) alkyl,

S—(C₃-C₆)cycloalkyl,

NH₂,

NH(C₁-C₄)alkyl,

N((C₁-C₄)alkyl)₂, or

NH—(C₃-C₆)cycloalkyl;

R₃ and R₄ independently are halo,

(C₁-C₈)alkyl,

(C₃-C₆)cycloalkyl,

O—(C₁-C₄)alkyl,

O—(C₃-C₆)cycloalkyl,

S—(C₁-C₄) alkyl,

S—(C₃-C₆)cycloalkyl,

NH₂,

NH(C₁-C₄)alkyl,

N((C₁-C₄)alkyl)₂,

NH—(C₃-C₆)cycloalkyl;

aryl,

(CH₂)ₙ-aryl,

heterocyclo,

(CH₂)ₙ-heterocyclo,

heteroaryl, or

(CH₂)ₙ-heteroaryl,

wherein n is 0, 1, 2, or 3;

R₅ is H,

(C₁-C₈)alkyl,

(C₃-C₆)cycloalkyl,

aryl,

(CH₂)ₙ-aryl,

heterocyclo,

(CH₂)ₙ-heterocyclo,

heteroaryl, or

(CH₂)ₙ-heteroaryl,

wherein n is as defined above.

What is also provided is a compound of formula III

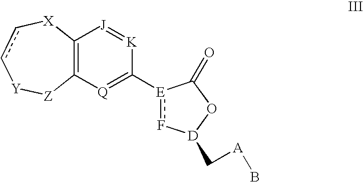

or a pharmaceutically acceptable salt thereof, wherein:

A is O,

NH, or

S;

B is C(═O)R₁,

C(═S)R₁,

heterocylco,

heteroaryl,

C(═O)-heterocyclo,

C(═N)—CN, or

C(═O)-heteteroaryl;

either D is N, E is C, and F is CH when “------” is a bond, or D is CH, E is N, and F is CH₂ when “------” is absent;

J, K, Q independently are CR₂ or N, with the proviso that when any one of J, K, or Q is N, then the other two are CR₂;

“------” is absent or is a bond;

X, Y, Z independently are C═C—R₅,

O═C,

CHR₃

CHR₄,

CR₃R₄,

NR₅,

N(C═O)R₅,

N(C═O)OR₅,

NSO₂R₅,

NSO₂NR₅,

O,

S,

SO, or

SO₂;

R₁ is H,

(C₁-C₈)alkyl,

(C₃-C₆)cycloalkyl,

O—(C₁-C₄)alkyl,

O—(C₃-C₆)cycloalkyl,

S—(C₁-C₄) alkyl,

S—(C₃-C₆)cycloalkyl,

NH₂,

NH(C₁-C₄)alkyl,

N((C₁-C₄)alkyl)₂, or

NH—(C₃-C₆)cycloalkyl;

R₂ is H,

halo,

(C₁-C₈)alkyl,

(C₃-C₆)cycloalkyl,

O—C₁-C₄)alkyl,

O—(C₃-C₆)cycloalkyl,

S—(C₁-C₄) alkyl,

S—(C₃-C₆)cycloalkyl,

NH₂,

NH(C₁-C₄)alkyl,

N((C₁-C₄)alkyl)₂, or

NH—(C₃-C₆)cycloalkyl;

R₃ and R₄ independently are H,

halo,

(C₁-C₈)alkyl,

(C₃-C₆)cycloalkyl,

O—(C₁-C₄)alkyl,

O—(C₃-C₆)cycloalkyl,

S—(C₁-C₄) alkyl,

S—(C₃-C₆)cycloalkyl,

NH₂,

NH(C₁-C₄)alkyl,

N((C₁-C₄)alkyl)₂,

NH—(C₃-C₆)cycloalkyl;

aryl,

(CH₂)ₙ-aryl,

heterocyclo,

(CH₂)ₙ-heterocyclo,

heteroaryl, or

(CH₂)ₙ-heteroaryl,

wherein n is 0, 1, 2, or 3;

R₅ is H,

(C₁-C₈)alkyl,

(C₃-C₆)cycloalkyl,

aryl,

(CH₂)aryl,

heterocyclo,

(CH₂)heterocyclo,

heteroaryl, or

(CH₂)heteroaryl,

wherein n is as defined above.

What is also provided is a compound of formula IV

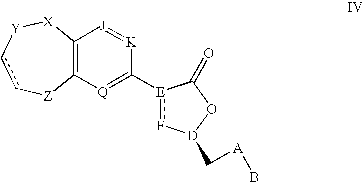

or a pharmaceutically acceptable salt thereof, wherein:

A is O,

NH, or

S;

B is

C(═O)R₁,

C(═S)R₁,

heterocylco,

heteroaryl,

C(═O)-heterocyclo,

C(═N)—CN, or

C(═O)-heteteroaryl;

either D is N, E is C, and F is CH when “------” is a bond, or D is CH, E is N, and F is CH₂ when “------” is absent;

J, K, Q independently are CR₂ or N, with the proviso that when any one of J, K, or Q is N, then the other two are CR₂;

“------” is absent; or is a bond; and

X, Y, Z independently are C═C—R₅,

O═C,

CH₂,

CHR₃,

CHR₄,

CR₃R₄,

NR₅,

N(C═O)R₅,

N(C═O)OR₅,

NSO₂R₅,

NSO₂NR₅,

O,

S,

SO, or

SO₂;

R₁ is H,

(C₁-C₈)alkyl,

(C₃-C₆)cycloalkyl,

O—(C₁-C₄)alkyl,

O—(C₃-C₆)cycloalkyl,

S—(C₁-C₄) alkyl,

S—(C₃-C₆)cycloalkyl,

NH₂,

NH(C₁-C₄)alkyl,

N((C₁-C₄)alkyl)₂, or

NH—(C₃-C₆)cycloalkyl,

R₂ is H,

halo,

(C₁-C₈)alkyl,

(C₃-C₆)cycloalkyl,

O—(C₁-C₄)alkyl,

O—(C₃-C₆)cycloalkyl,

S—(C₁-C₄) alkyl,

S—(C₃-C₆)cycloalkyl,

NH₂,

NH(C₁-C₄)alkyl,

N((C₁-C₄)alkyl)₂, or

NH—(C₃-C₆)cycloalkyl;

R₃ and R₄ independently are halo,

(C₁-C₈)alkyl,

(C₃-C₆)cycloalkyl,

O—(C₁-C₄)alkyl,

O—(C₃-C₆)cycloalkyl,

S—(C₁-C₄) alkyl,

S—(C₃-C₆)cycloalkyl,

NH₂,

NH(C₁-C₄)alkyl,

N((C₁-C₄)alkyl)₂,

NH—(C₃-C₆)cycloalkyl;

aryl,

(CH₂)ₙ-aryl,

heterocyclo,

(CH₂)ₙ-heterocyclo,

heteroaryl, or

(CH₂)ₙ-heteroaryl,

wherein n is 0, 1, 2, or 3;

R₅ is H,

(C₁-C₈)alkyl,

(C₃-C₆)cycloalkyl,

aryl,

(CH₂)ₙ-aryl,

heterocyclo,

(CH₂)ₙ-heterocyclo,

heteroaryl, or

(CH₂)ₙ-heteroaryl,

wherein n is as defined above.

What is also provided is a compound of formula V

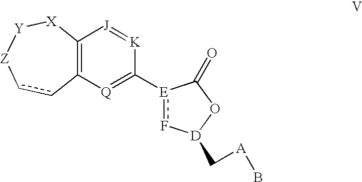

or a pharmaceutically acceptable salt thereof wherein:

A is O,

NH, or

S;

B is

C(═O)R₁,

C(═S)R₁,

heterocylco,

heteroaryl,

C(═O)-heterocyclo,

C(═N)—CN, or

C(═O)-heteteroaryl;

either D is N, E is C, and F is CH when “------” is a bond, or D is CH, E is N, and F is CH₂ when “------” is absent;

J, K, Q independently are CR₂ or N, with the proviso that when any one of J, K, or Q is N, then the other two are CR₂;

“------” is absent; or is a bond; and

X, Y, Z independently are C═C—R₅,

O═C,

CH₂,

CHR₃,

CHR₄,

CR₃R₄,

NR₅,

N(C═O)R₅,

N(C═O)OR₅,

NSO₂R₅,

NSO₂NR₅,

O,

S,

SO, or

SO₂;

R₁ is H,

(C₁-C₈)alkyl,

(C₃-C₆)cycloalkyl,

O—(C₁-C₄)alkyl,

O—(C₃-C₆)cycloalkyl,

S—(C₁-C₄) alkyl,

S—(C₃-C₆)cycloalkyl,

NH₂,

NH(C₁-C₄)alkyl,

N((C₁-C₄)alkyl)₂, or

NH—(C₃-C₆)cycloalkyl,

R₂ is H,

halo,

(C₁-C₈)alkyl,

(C₃-C₆)cycloalkyl,

O—(C₁-C₄)alkyl,

O—(C₃-C₆)cycloalkyl,

S—(C₁-C₄) alkyl,

S—(C₃-C₆)cycloalkyl,

NH₂,

NH(C₁-C₄)alkyl,

N((C₁-C₄)alkyl)₂, or

NH—(C₃-C₆)cycloalkyl;

R₃ and R₄ independently are halo,

(C₁-C₈)alkyl,

(C₃-C₆)cycloalkyl,

O—(C₁-C₄)alkyl,

O—(C₃-C₆)cycloalkyl,

S—(C₁-C₄) alkyl,

S—(C₃-C₆)cycloalkyl,

NH₂,

NH(C₁-C₄)alkyl,

N((C₁-C₄)alkyl)₂,

NH—(C₃-C₆)cycloalkyl;

aryl,

(CH₂)ₙ-aryl,

heterocyclo,

(CH₂)ₙ-heterocyclo,

heteroaryl, or

(CH₂)ₙ-heteroaryl,

wherein n is 0, 1, 2, or 3;

R₅ is H,

(C₁-C₈)alkyl,

(C₃-C₆)cycloalkyl,

aryl,

(CH₂)ₙ-aryl,

heterocyclo,

(CH₂)ₙ-heterocyclo,

heteroaryl, or

(CH₂)ₙ-heteroaryl,

wherein n is as defined above.

What is also provided is a compound which is:

(S)-N-[2-Oxo-3-(6,7,8,9-tetrahydro-5H-benzocyclohepten-2-yl)-oxazolidin-5-ylmethyl]-acetamide;

(S)-N-[2-Oxo-3-(5-oxo-6,7,8,9-tetrahydro-5H-benzocyclohepten-2-yl)-oxazolidin-5-ylmethyl]-acetamide;

(S)-N-[3-(6-Bromo-5-oxo-6,7,8,9-tetrahydro-5H-benzocyclohepten-2-yl)-2-oxo-oxazolidin-5-ylmethyl]-acetamide;

(S)-N-[3-(6-Dimethylaminomethylene-5-oxo-6,7,8,9-tetrahydro-5H-benzocyclohepten-2-yl)-2-oxo-oxazolidin-5-ylmethyl]-acetamide;

(S)-N-[2-Oxo-3-(5-oxo-6-pyridin-4-ylmethylene-6,7,8,9-tetrahydro-5H-benzocyclohepten-2-yl)-oxazolidin-5-ylmethyl]-acetamide;

(S)-N-[3-(6-Benzylidene-5-oxo-6,7,8,9-tetrahydro-5H-benzocyclohepten-2-yl)-2-oxo-oxazolidin-5-ylmethyl]-acetamide;

(S)-N-{3-[6-(4-Fluoro-benzylidene)-5-oxo-6,7,8,9-tetrahydro-5H-benzocyclohepten-2-yl]-2-oxo-oxazolidin-5-ylmethyl}-acetamide;

(S)-N-[2-Oxo-3-(5-oxo-6-thiophen-3-ylmethylene-6,7,8,9-tetrahydro-5H-benzocyclohepten-2-yl)-oxazolidin-5-ylmethyl]-acetamide;

(S)-N-[3-(6-Furan-3-ylmethylene-5-oxo-6,7,8,9-tetrahydro-5H-benzocyclohepten-2-yl)-2-oxo-oxazolidin-5-ylmethyl]-acetamide;

(S)-N-[2-Oxo-3-(6-oxo-6,7,8,9-tetrahydro-5H-benzocyclohepten-2-yl)-oxazolidin-5-ylmethyl]-acetamide;

(S)-N-[2-Oxo-3-(7-oxo-6,7,8,9-tetrahydro-5H-benzocyclohepten-2-yl)-oxazolidin-5-ylmethyl]-acetamide;

(S)-N-[2-Oxo-3-(8-oxo-6,7,8,9-tetrahydro-5H-benzocyclohepten-2-yl)-oxazolidin-5-ylmethyl]-acetamide;

(S)-N-[2-Oxo-3-(9-oxo-6,7,8,9-tetrahydro-5H-benzocyclohepten-2-yl)-oxazolidin-5-ylmethyl]-acetamide;

(S)-N-[3-(8,9-Dihydro-7H-benzocyclohepten-2-yl)-2-oxo-oxazolidin-5-ylmethyl]-acetamide;

(S)-N-[3-(8,9-Dihydro-5H-benzocyclohepten-2-yl)-2-oxo-oxazolidin-5-ylmethyl]-acetamide;

(S)-N-[3-(6,9-Dihydro-5H-benzocyclohepten-2-yl)-2-oxo-oxazolidin-5-ylmethyl]-acetamide;

(S)-N-[3-(6,7-Dihydro-5H-benzocyclohepten-2-yl)-2-oxo-oxazolidin-5-ylmethyl]-acetamide;

(S)-N-[2-Oxo-3-(2,3,4,5-tetrahydro-1H-benzo[b] azepin-7-yl)-oxazolidin-5-ylmethyl]-acetamide;

(S)-N-[2-Oxo-3-(2,3,4,5-tetrahydro-1H-benzo[c]azepin-7-yl)-oxazolidin-5-ylmethyl]-acetamide;

(S)-N-[2-Oxo-3-(2,3,4,5-tetrahydro-1H-benzo[d]azepin-7-yl)-oxazolidin-5-ylmethyl]-acetamide;

(S)-N-[2-Oxo-3-(2,3,4,5-tetrahydro-1H-benzo[c]azepin-8-yl)-oxazolidin-5-ylmethyl]-acetamide;

(S)-N-[2-Oxo-3-(2,3,4,5-tetrahydro-1H-benzo[b]azepin-8-yl)-oxazolidin-5-ylmethyl]-acetamide;

(S)-N-[2-Oxo-3-(2,3,4,5-tetrahydro-benzo[b]oxepin-7-yl)-oxazolidin-5-ylmethyl]-acetamide;

(S)-N-[2-Oxo-3-(1,3,4,5-tetrahydro-benzo[c]oxepin-7-yl)-oxazolidin-5-ylmethyl]-acetamide;

(S)-N-[2-Oxo-3-(5,6,8,9-tetrahydro-7-oxa-benzocyclohepten-2-yl)-oxazolidin-5-ylmethyl]-acetamide;

(S)-N-[2-Oxo-3-(1,3,4,5-tetrahydro-benzo[c]oxepin-8-yl)-oxazolidin-5-ylmethyl]-acetamide;

(S)-N-[2-Oxo-3-(2,3,4,5-tetrahydro-benzo[b]oxepin-8-yl)-oxazolidin-5-ylmethyl]-acetamide;

(S)-N-[2-Oxo-3-(6,7,8,9-tetrahydro-5-oxa-7-aza-benzocyclohepten-2-yl)-oxazolidin-5-ylmethyl]-acetamide;

(S)-N-[2-Oxo-3-(2,3,4,5-tetrahydro-benzo[b]thiepin-7-yl)-oxazolidin-5-ylmethyl]-acetamide;

(S)-N-[2-Oxo-3-(1,3,4,5-tetrahydro-benzo[c]thiepin-7-yl)-oxazolidin-5-ylmethyl]-acetamide;

(S)-N-[2-Oxo-3-(1,2,4,5-tetrahydro-benzo[d]thiepin-7-yl)-oxazolidin-5-ylmethyl]-acetamide;

(S)-N-[2-Oxo-3-(1,3,4,5-tetrahydro-benzo[c]thiepin-8-yl)-oxazolidin-5-ylmethyl]-acetamide;

(S)-N-[2-Oxo-3-(2,3,4,5-tetrahydro-benzo[b]thiepin-8-yl)-oxazolidin-5-ylmethyl]-acetamide;

(S)-N-[2-Oxo-3-(5-oxo-2,3,4,5-tetrahydro-benzo[b]oxepin-8-yl)-oxazolidin-5-ylmethyl]-acetamide;

(S)-N-[2-Oxo-3-(5-oxo-2,3,4,5-tetrahydro-benzo[b]thiepin-8-yl)-oxazolidin-5-ylmethyl]-acetamide;

(S)-N-[2-Oxo-3-(1,1,5-trioxo-2,3,4,5-tetrahydro-1H-116-benzo[b]thiepin-8-yl)-oxazolidin-5-ylmethyl]-acetamide;

(S)-N-[2-Oxo-3-(5-oxo-2,3,4,5-tetrahydro-benzo[b]oxepin-7-yl)-oxazolidin-5-ylmethyl]-acetamide;

(S)-N-[3-(6,6-Difluoro-5-oxo-6,7,8,9-tetrahydro-5H-benzocyclohepten-2-yl)-2-oxo-oxazolidin-5-ylmethyl]-acetamide;

(S)-N-[3-(6-Benzylidene-5-oxo-6,7,8,9-tetrahydro-5H-benzocyclohepten-2-yl)-2-oxo-oxazolidin-5-ylmethyl]-acetamide;

(S)-N-[3-(2-Methyl-2,3,4,5-tetrahydro-1H-benzo[c]azepin-7-yl)-2-oxo-oxazolidin-5-ylmethyl]-acetamide; or

(S)-N-[3-(3-Methyl-2,3,4,5-tetrahydro-1H-benzo[d]azepin-7-yl)-2-oxo-oxazolidin-5-ylmethyl]-acetamide.

What is also provided is a pharmaceutical formulation comprising a compound of one of formulas I-V admixed with a pharmaceutically acceptable diluent, carrier, or excipient.

What is also provided is a method of treating a bacterial infection in a mammal, comprising administering to a mammal in need thereof an effective amount of a compound of one of formulas I-V.

### DETAILED DESCRIPTION OF THE INVENTION

Reference will now be made in detail to presently preferred compositions or embodiments and methods of the invention, which constitute the best modes of practicing the invention presently known to the inventors.

The term “alkyl” as used herein refers to a straight or branched hydrocarbon of from 1 to 8 carbon atoms and includes, for example, methyl, ethyl, n-propyl, isopropyl, n-butyl, sec-butyl, isobutyl, tert-butyl, n-pentyl, n-hexyl, and the like. The alkyl group can also be substituted with one or more of the substituents selected from lower alkoxy, lower thioalkoxy, halogen, nitro, cyano, oxo, thio, —OH, —SH, —F, —CF₃, —OCF₃, —NO₂, —CO₂H, —CO₂C₁-C₆ alkyl, —NH₂, —NHC₁-C₆ alkyl,

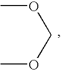

CONR⁸R⁹, or —N(C₁-C₆alkyl)₂. Preferred alkyl groups have from 1 to 6 carbon atoms (C₁-C₆ alkyl).

The terms “(C₁-C₈)alkyl”, “(C₁-C₆)alkyl”, and “(C₁-C₄)alkyl” as used herein refer to subsets of alkyl which mean a straight or branched hydrocarbon radical having from 1 to 8, 1 to 6, or 1 to 4 carbon atoms respectivly, and include, for example, methyl, ethyl, n-propyl, isopropyl, n-butyl, sec-butyl, isobutyl, tert-butyl, n-pentyl, n-hexyl, n-heptyl, and n-octyl and the like.

The term “(C₃-C₆)cycloalkyl” means a hydrocarbon ring containing from 3 to 6 carbon atoms, for example, cyclopropyl, cyclobutyl, cyclopentyl, or cyclohexyl. Where possible, the cycloalkyl group may contain double bonds, for example, 3-cyclohexen-1-yl. The cycloalkyl ring may be unsubstituted or substituted by one or more substituents selected from alkyl, alkoxy, thioalkoxy, hydroxy, thiol, nitro, halogen, amino, alkyl and dialkylamino, formyl, carboxyl, CN, —NH—CO—R, —CO—NHR, —CO₂R, —COR, wherein R is defined as above, aryl, heteroaryl, wherein alkyl, aryl, and heteroaryl are as defined herein, or as otherwise indicated above for alkyl, alkenyl, and alkynyl substitutents. Examples of substituted cycloalkyl groups include fluorocyclopropyl, 2-iodocyclobutyl, 2,3-dimethylcyclopentyl, 2,2-dimethoxycyclohexyl, and 3-phenylcyclopentyl.

The term “halo” includes chlorine, fluorine, bromine, and iodine.

The term “aryl” means a cyclic or polycyclic aromatic ring having from 5 to 12 carbon atoms, and being unsubstituted or substituted with one or more of the substituent groups recited above for alkyl groups including, halogen, nitro, cyano —OH, —SH, —F, —CF₃, —OCF₃, —NO₂,

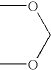

CO₂H, —CO₂C₁-C₆ alkyl, —NH₂, —NHC₁-C₆ alkyl, —CONR^{a}R^{b}, wherein R^{a} and R^{b} are H or (C₁-C₆)alkyl or (C₃-C₆)cycloalkyl, SO₂alkyl, —SO₂NH₂, or —N(C₁-C₆alkyl)₂. Examples include, but are not limited to phenyl, 2-chlorophenyl, 3-chlorophenyl, 4-chlorophenyl, 2-methylphenyl, 3-methylphenyl, 4-methylphenyl, 2-methoxyphenyl, 3-methoxyphenyl, 4-methoxyphenyl, 2-chloro-3-methylphenyl, 2-chloro-4-methylphenyl, 2-chloro-5-methylphenyl, 3-chloro-2-methylphenyl, 3-chloro-4-methylphenyl, 4-chloro-2-methylphenyl, 4-chloro-3-methylphenyl, 5-chloro-2-methylphenyl, 2,3-dichlorophenyl, 2,5-dichlorophenyl, 3,4-dichlorophenyl, 2,3-dimethylphenyl, 3,4-dimethylphenyl, thienyl, naphthyl, 4-thionaphthyl, tetralinyl, anthracinyl, phenanthrenyl, benzonaphthenyl, fluorenyl, 2-acetamidofluoren-9-yl, and 4′-bromobiphenyl.

The term “heteroaryl” means an aromatic cyclic or polycyclic ring system having from 1 to 4 heteroatoms selected from N, O, and S. Typical heteroaryl groups include 2- or 3-thienyl, 2- or 3-furanyl, 2- or 3-pyrrolyl, 2-, 4-, or 5-imidazolyl, 3-, 4-, or 5-pyrazolyl, 2-, 4-, or 5-thiazolyl, 3-, 4-, or 5-isothiazolyl, 2-, 4-, or 5-oxazolyl, 3-, 4-, or 5-isoxazolyl, 3- or 5-1,2,4-triazolyl, 4- or 5-1,2,3-triazolyl, tetrazolyl, 2-, 3-, or 4-pyridinyl, 3-, 4-, or 5-pyridazinyl, 2-pyrazinyl, 2-, 4-, or 5-pyrimidinyl, 2-, 3-, 4-, 5-, 6-, 7-, or 8-quinolinyl, 1-, 3-, 4-, 5-, 6-, 7-, or 8-isoquinolinyl, 2-, 3-, 4-, 5-, 6-, or 7-indolyl, 2-, 3-, 4-, 5-, 6-, or 7-benzo[b]thienyl, 2-, 4-, 5-, 6-, or 7-benzoxazolyl, 2-, 4-, 5-, 6-, or 7-benzimidazolyl, 2-, 4-, 5-, 6-, or 7-benzothiazolyl. The heteroaryl groups may be unsubstituted or substituted by 1 to 3 substituents selected from those described above for alkyl, alkenyl, and alkynyl, for example, cyanothienyl and formylpyrrolyl. Preferred aromatic fused heterocyclic rings of from 8 to 10 atoms include but are not limited to 2-, 3-, 4-, 5-, 6-, 7-, or 8-quinolinyl, 1-, 3-, 4-, 5-, 6-, 7-, or 8-isoquinolinyl-, 2-, 3-, 4-, 5-, 6-, or 7-indolyl, 2-, 3-, 4-, 5-, 6-, or 7-benzo[b]thienyl, 2-, 4-, 5-, 6-, or 7-benzoxazolyl, 2-, 4-, 5-, 6-, or 7-benzimidazolyl, 2-, 4-, 5-, 6-, or 7-benzothiazolyl. Heteroaryl also includes 2- and 3-aminomethylfuran, 2- and 3-aminomethylthiophene and the like.

The term “heterocyclic” means a monocyclic, fused, bridged, or spiro bicyclic heterocyclic ring systems. Monocyclic heterocyclic rings contain from about 3 to 12 ring atoms, with from 1 to 5 heteroatoms selected from N, O, and S, and preferably from 3 to 7 member atoms, in the ring. Bicyclic heterocyclics contain from about 5 to about 17 ring atoms, preferably from 5 to 12 ring atoms. Bicyclic heterocyclic rings may be fused, spiro, or bridged ring systems. Examples of heterocyclic groups include cyclic ethers (oxiranes) such as ethyleneoxide, tetrahydrofuran, dioxane, and substituted cyclic ethers, wherein the substituents are those described above for the alkyl and cycloalkyl groups. Typical substituted cyclic ethers include propyleneoxide, phenyloxirane (styrene oxide), cis-2-butene-oxide (2,3-dimethyloxirane), 3-chlorotetrahydrofuran, 2,6-dimethyl-1,4-dioxane, and the like. Heterocycles containing nitrogen are groups such as pyrrolidine, piperidine, piperazine, tetrahydrotriazine, tetrahydropyrazole, and substituted groups such as 3-aminopyrrolidine, 4-methylpiperazin-1-yl, and the like. Typical sulfur containing heterocycles include tetrahydrothiophene, dihydro-1,3-dithiol-2-yl, and hexahydrothiophen-4-yl and substituted groups such as aminomethyl thiophene. Other commonly employed heterocycles include dihydro-oxathiol-4-yl, dihydro-1H-isoindole, tetrahydro-oxazolyl, tetrahydro-oxadiazolyl, tetrahydrodioxazolyl, tetrahydrooxathiazolyl, hexahydrotriazinyl, tetrahydro-oxazinyl, morpholinyl, thiomorpholinyl, tetrahydropyrimidinyl, dioxolinyl, octahydrobenzofuranyl, octahydrobenzimidazolyl, and octahydrobenzothiazolyl. For heterocycles containing sulfur, the oxidized sulfur heterocycles containing SO or SO₂ groups are also included. Examples include the sulfoxide and sulfone forms of tetrahydrothiophene.

When a bond is represented by a line such as “------” this is meant to represent that the bond may be absent or present, provided that the resultant compound is stable and of satisfactory valency.

The term “patient” means all mammals, including humans. Other examples of patients include cows, dogs, cats, goats, sheep, pigs, and rabbits.

A “therapeutically effective amount” is an amount of a compound of the present invention that when administered to a patient, elicits the desired therapeutic outcome; i.e., inhibits bacterial infection.

It will be appreciated by those skilled in the art that compounds of the invention having one or more chiral centers may exist in and be isolated in optically active and racemic forms. Some compounds may exhibit polymorphism. It is to be understood that the present invention encompasses any racemic, optically-active, polymorphic, geometric, or stereoisomeric form, or mixtures thereof, of a compound of the invention, which possess the useful properties described herein, it being well known in the art how to prepare optically active forms (for example, by resolution of the racemic form by recrystallization techniques, by synthesis from optically-active starting materials, by chiral synthesis, or by chromatographic separation using a chiral stationary phase) and how to determine activity or cytotoxicity using the standard tests described herein, or using other similar tests which are well known in the art.

A “prodrug” is an inactive derivative of a drug molecule that requires a chemical or an enzymatic biotransformation in order to release the active parent drug in the body.

Specific and preferred values for compounds of Formula I are listed below for radicals, substituents, and ranges are for illustration purposes only, and they do not exclude other defined values or other values within defined ranges for the radicals and substituents.

Thus, turning now to a compound of formula I, a specific value for A is NH, as designated in formula IA.

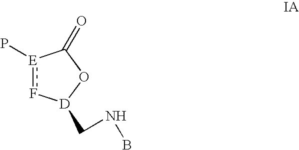

A specific value for B is acetyl as designated in formula IB.

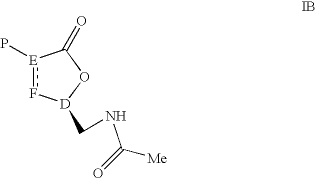

Specific values for D, E, and F are as designated in formula IC.

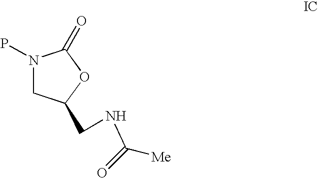

In one embodiment, a specific values for P is

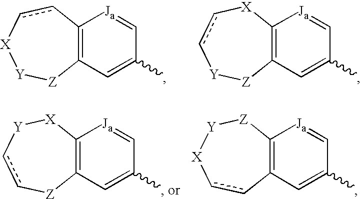

wherein “ [symbol] ” indicates the point of attachment, and Jₐ is N or CR′, wherein R′ is H or F

In another embodiment, a specific value for P is

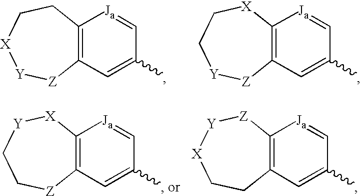

In another embodiment, two of X, Y, or Z in P is C═C—R₅,

O═C,

NR₅,

N(C═O)R₅,

N(C═O)OR₅,

NSO₂R₅,

NSO₂NR₅,

O,

S,

SO, or

SO₂NR₅,

and the other of X, Y, or Z is CH₂ or CR₃R₄.

In another embodiment, P is

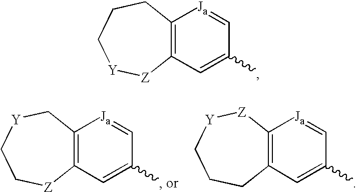

In another embodiment, P is

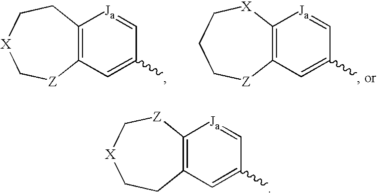

In another embodiment, P is

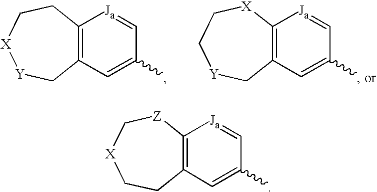

In still another embodiment, one of X, Y, or Z is C═C—R₅,

O═C,

NR₅,

N(C═O)R₅,

N(C═O)OR₅,

NSO₂R₅,

NSO₂NR₅,

O,

S,

SO, or

SO₂NR₅,

and the others of X, Y, or Z is CH₂.

In yet another embodiment, P is

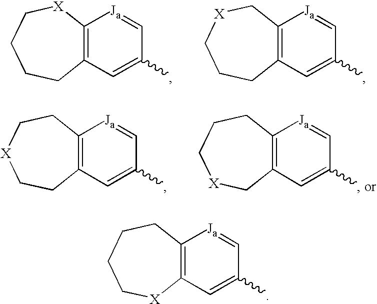

Turning now to a compound of formula II., a specific value for A is NH, as designated in formula IIA.

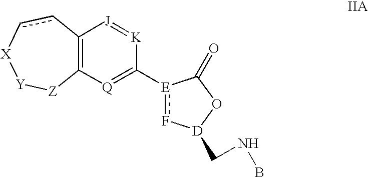

A specific value for B is acetyl, as designated in formula IIB.

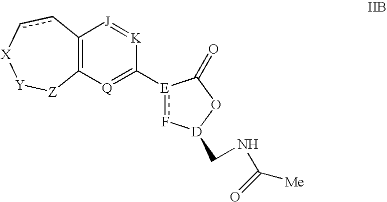

Specific values for D, E, and F, are CH, N, and CH₂, respectively, as designated in formula IIC.

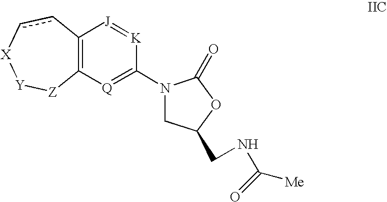

As designated in formula IID, a specific value for J is Ja, wherein Jₐ is N or CR′, and wherein R′ is H or F.

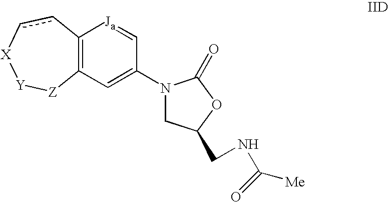

As designated in formula IIE, “------” is absent.

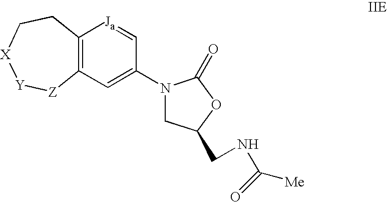

In one embodiment, in a compound of formula IIE, two of X, Y, or Z is

C═C—R₅,

O═C,

NR₅,

N(C═O)R₅,

N(C═O)OR₅,

NSO₂R₅,

NSO₂NR₅,

O,

S,

SO, or

SO₂NR₅,

and the other of X, Y, or Z is CH₂ or CR₃R₄.

In another embodiment, specific values for X, Y, and Z are as designated in formula IIF.

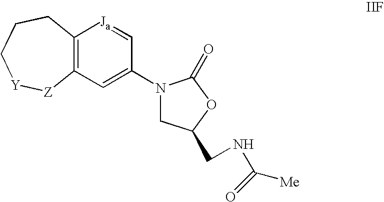

In another embodiment, specific values for X, Y, and Z are as designated in formula IIG.

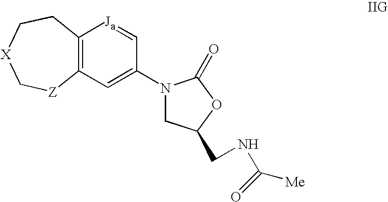

In another embodiment, specific values for X, Y, and Z are as designated in formula IIH.

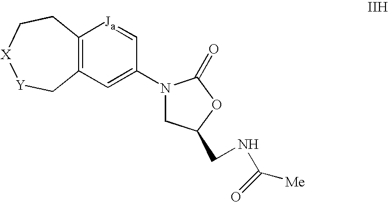

In another embodiment, one of X, Y, or Z is C═C—R₅,

O═C,

NR₅,

N(C═O)R₅,

N(C═O)OR₅,

NSO₂R₅,

NSO₂NR₅,

O,

SO, or

SO₂NR₅,

and the others of X, Y, or Z is CH₂.

Turning now to a compound of formula III, a specific value for A is NH, as designated in formula IIIA.

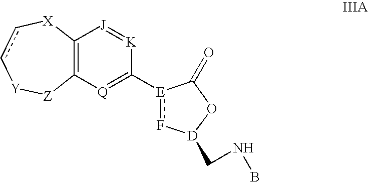

A specific value for B is acetyl, as designated in formula IIIB.

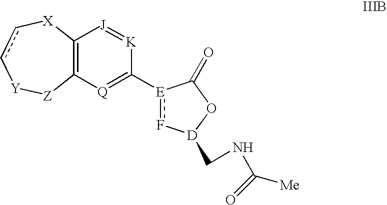

Specific values for D, E, and F, are CH, N, and CH₂, respectively, as designated in formula IIIC.

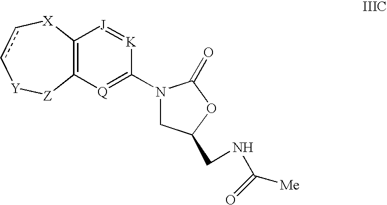

As designated in formula IIID, a specific value for J is Ja, wherein Jₐ is N or CR′, and wherein R′ is H or F.

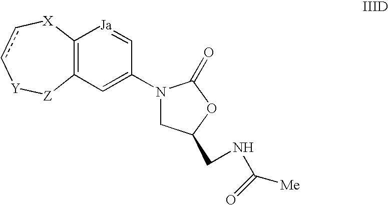

As designated in formula IIIE, “------” is absent.

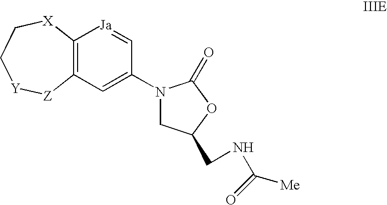

In one embodiment, in a compound of formula IIIE, two of X, Y, or Z is

C═C—R₅,

O═C,

NR₅,

N(C═O)R₅,

N(C═O)OR₅,

NSO₂R₅,

NSO₂NR₅,

O,

S,

SO, or

SO₂NR₅,

and the other of X, Y, or Z is CH₂ or CR₃R₄.

In another embodiment, specific values for X, Y, and Z are as designated in formula IIIF.

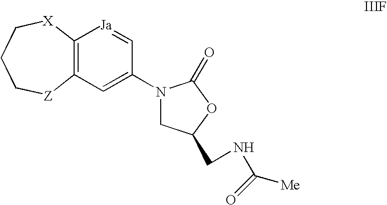

In another embodiment, specific values for X, Y, and Z are as designated in formula IIH.

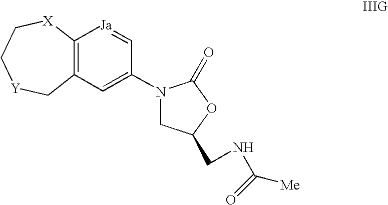

In another embodiment, one of X, Y, or Z is C═C—R₅,

O═C,

NR₅,

N(C═O)R₅,

N(C═O)OR₅,

NSO₂R₅,

NSO₂NR₅,

O,

S,

SO, or

SO₂NR₅,

and the other of X, Y, or Z is CH₂.

Turning now to a compound of formula IV, a specific value for A is NH as designated in formula IVA.

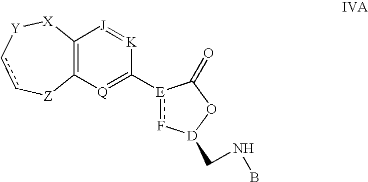

A specific value for B is acetyl as designated in formula IVB.

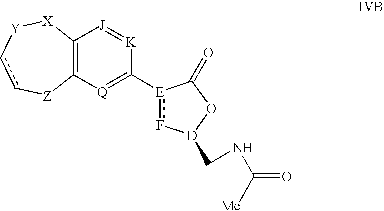

Specific values for D, E, and F, are CH, N, and CH₂, respectively, as designated in formula IVC.

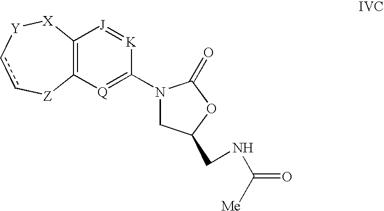

As designated in formula IVD, a specific value for J is Ja, wherein Jₐ is N or CR′, and wherein R′ is H or F.

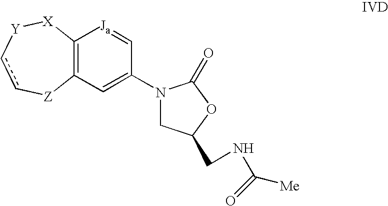

As designated in formula IVE, “------” is absent.

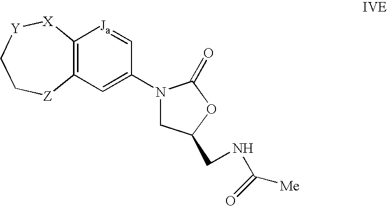

In one embodiment, in a compound of formula IVE, two of X, Y, or Z is

C═C—R₅,

O═C,

NR₅,

N(C═O)R₅,

N(C═O)OR₅,

NSO₂R₅,

NSO₂NR₅,

O,

S,

SO, or

SO₂NR₅,

and the other of X, Y, or Z is CR₃R₄.

In another embodiment, specific values for X, Y, and Z are as designated in formula IVF.

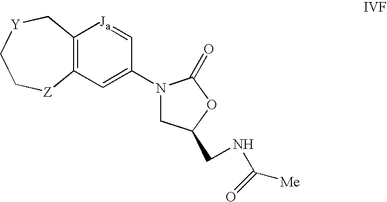

In another embodiment, specific values for X, Y, and Z are as designated in formula IVG.

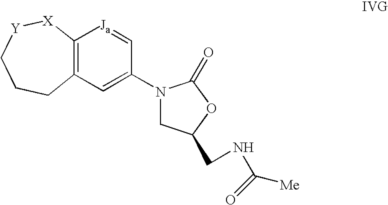

In another embodiment, one of X, Y, or Z is C═C—R₅,

O═C,

NR₅,

N(C═O)R₅,

N(C═O)OR₅,

NSO₂R₅,

NSO₂NR₅,

O,

S,

SO, or

SO₂NR₅,

and the other of X, Y, or Z is CH₂.

Turning now to a compound of formula V, a specific value for A is NH as designated in formula VA.

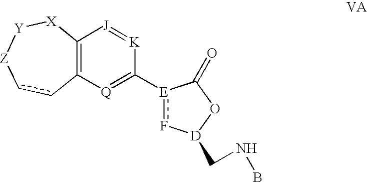

A specific value for B is acetyl as designated in formula VB.

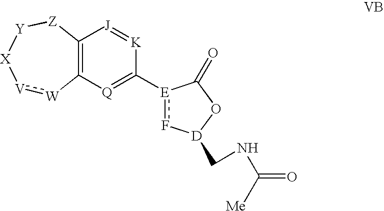

Specific values for D, E, and F, are CH, N, and CH₂, respectively, as designated in formula VC.

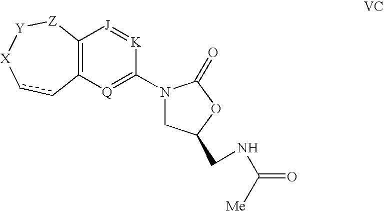

As designated in formula VD, a specific value for J is Ja, wherein Jₐ is N or CR′, and wherein R′ is H or F.

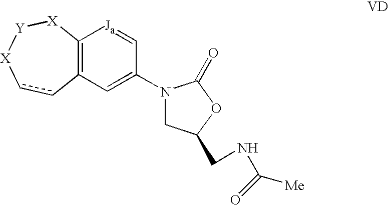

As designated in formula VE, “------” is absent.

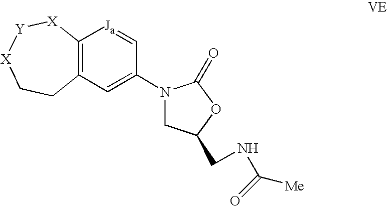

In one embodiment, in a compound of formula VE, two of X, Y, or Z is

C═C—R₅,

O═C,

NR₅,

N(C═O)R₅,

N(C═O)OR₅,

NSO₂R₅,

NSO₂NR₅,

O,

S,

SO, or

SO₂NR₅,

and the other of X, Y, or Z is CR₃R₄.

In another embodiment, specific values for X, Y, and Z are as designated in formula IVF.

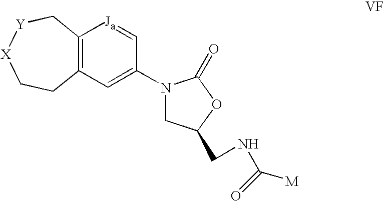

In another embodiment, specific values for X, Y, and Z are as designated in formula IVG.

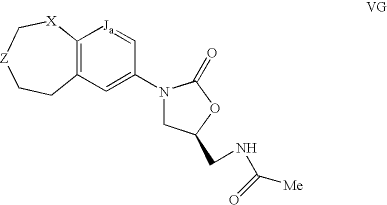

In another embodiment, specific values for X, Y, and Z are as designated in formula VH.

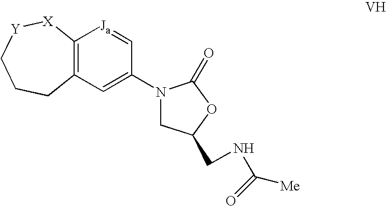

### Preparation of Invention Compounds

As is readily apparent from this disclosure, compounds of the present invention are characterized by a fused bicyclic subunit, covalently attached to a oxazolidinyl subunit. As retrosynthetically depicted in Scheme I, the invention compounds can be prepared from the corresponding benzocycloheptane via coupling procedures (D) available to the skilled artisan employing the oxazolidinone subunit itself or a synthon thereof. The requisite benzocyclohptyl compounds can be accessed via (A) annelation; (B) elaboration of a commercially available benzocycloheptane (B); or (C) ring expansion of a substituted di-tetrahydro naphthalene.

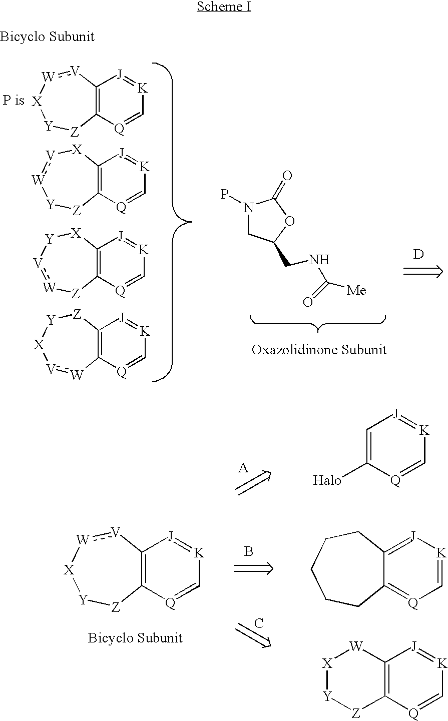

Reflecting the synthetic strategies summarized in Scheme I, the following section describing the preparation of the invention compounds has two sections. The first section summarizes the preparation of common intermediates (for instance, the oxazolidinone core). The second section summarizes the preparation and attachment of bicyclo subunits to the oxazolidinyl core to from the bicyclo oxazolidinone intermediates.

1. Preparation of Common Intermediates

The following compounds which were used in the synthesis of the compounds of the invention, were prepared as follows.

(R)-5-Hydroxymethyl-oxazolidin-2-one

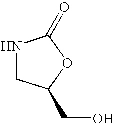

The title compound was prepared according to the procedure described by K. Danielmeier and E. Steckhan in Tetrahedron Assymetry 1995, 6(5), 1181-1190.

N-(2,4-Dimethoxy-benzyl)-N-(2-oxo-oxazolidin-5-ylmethyl)-acetamide

The title compound was prepared as described in Tetrahedron Letters, 2001, 42, 3681.

(S)-N-Oxiranylmethyl-acetamide

To a solution of (S)-N-acetyl-3-bromo-2-acetoxypropylamine (5 g, 0.021 mmol) in acetonitrile (20 mL) and methanol (20 mL) was added potassium carbonate (0.021 mmol) portion-wise. The reaction mixture was stirred at 0° C. for 1 hour and then warmed to room temperature slowly and stirred overnight. To it 50 mL of ethyl acetate was added and the precipitate was removed by filtration. Organic solvents were removed and the residue was dissolved in 60 mL of ethyl acetate and remaining precipitate was filtered and organic solution was concentration under reduced pressure to yield 1.6 g (90% yield) to obtain the title compound.

2. Preparation of Bicyclo-containing Oxazolidinones

A. Compounds with Ketone-Containing Benzocycloheptyl Cores

Strategies for the preparation of the bicyclo-containing oxazolidinones are depicted in the following section. Schemes 1A-D summarize the preparation of ketone-containing benzocycloheptyl cores. Thus, in Scheme 1A, nitration of bicyclo cycloheptanone 1A-1 (step I) provides nitro compound 1A-2, which is subsequently reduced to the amine 1A-3 (step II). Protection of the amine moiety in 1A-3 (step III), followed by treatment with (R)-gycidol butyrate provides oxazolidinone 1A-5 (step IV). Mesylation of the alcohol moiety in 1A-5 (step V), followed by treatment with sodium azide, provides azide 1A-7 (step VI). Hydrogenation (step VII) and acetylation (step VII) provides the target compound 1A-9

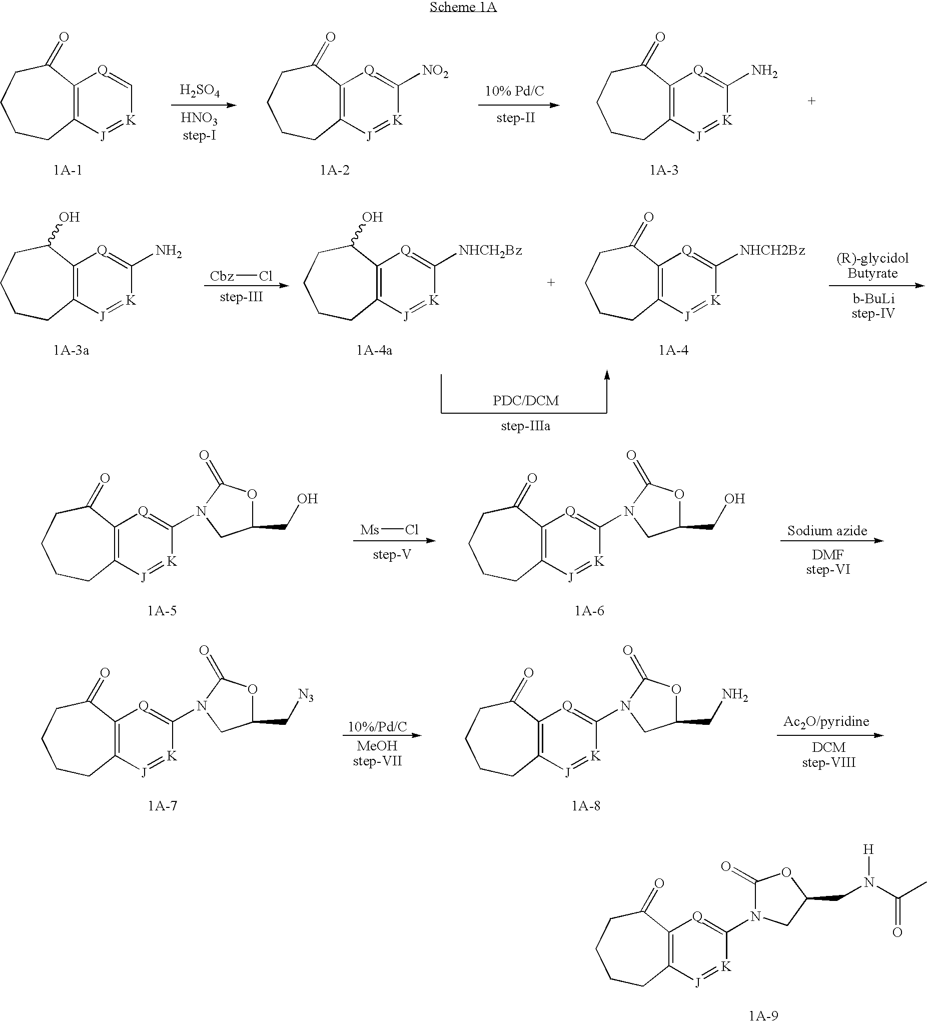

Scheme 1B provides a variant of the Scheme 1A approach, wherein the keto moiety is “walked” around the ring. Nitration of ketone 1B-1 (step I) provides nitro compound 1B-2, which is reduced to the corresponding amine 1B-3 (step II) under conditions known to the skilled artisan. Protection of the amine moiety (step III), followed by attachment of the oxazolidinone core using reagfents known to the skilled artisan provides 1B-5. Elaboration of the acetamide sidechain of the oxazolidinone subunit in 1B-5 commences with formation of the mesylate or an equivalent (step VI), followed by displacmement with azide, reduction (step VII) and acetylation (step VIII) to provide the target compound 1B-9.

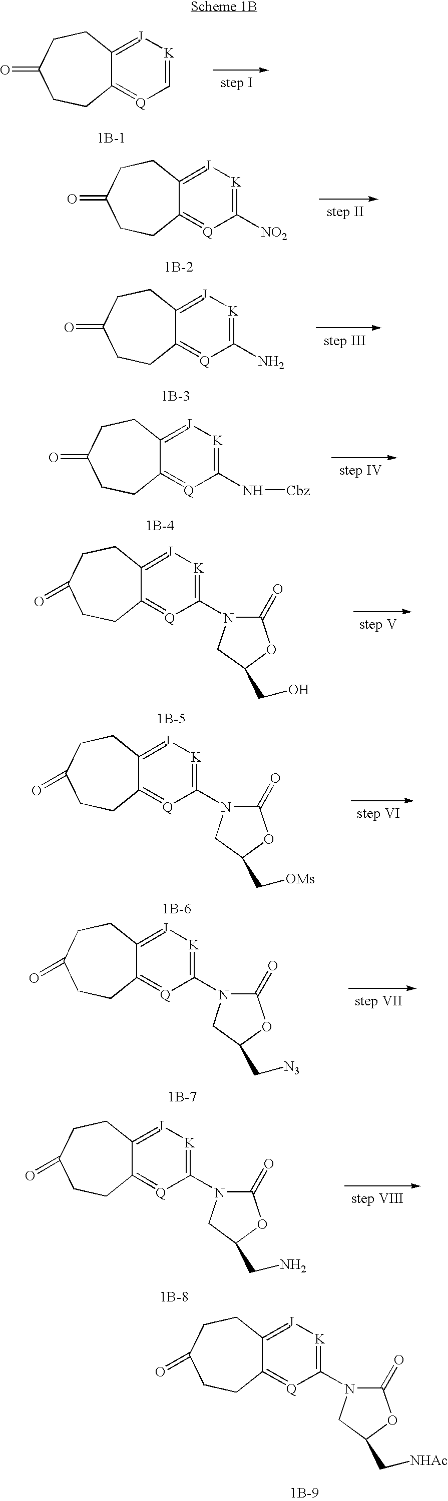

Scheme 1C provides another variant of the Scheme 1A approach. Thus, the keto moiety in compound 1C 1 is converted to the exo methylene compound 1C-2 (step I). Epoxidation and ring enlargement of 1C-2 affords ketone 1C-3. Coupling of compound 1C-2 to the oxazolidinone subunit (step III) provides 1C-4. Elaboration of the acetamide sidechain of the oxazolidinone subunit is as provided in Scheme 1B.

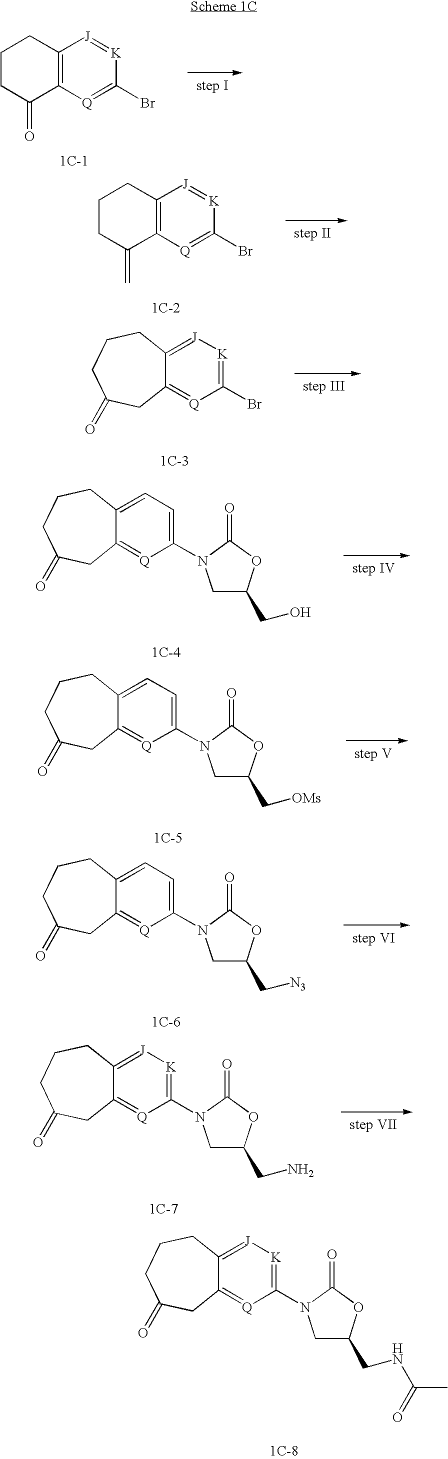

Scheme 1D provides a variant of the Scheme 1C approach. Thus, deprotection and bromination of ID-1 (step I) provides compound 1D-2. Steps II and III are similar to steps II and III in Scheme 1C. Coupling (step IV) and deprotection (step V) provide the target compound 1D-6.

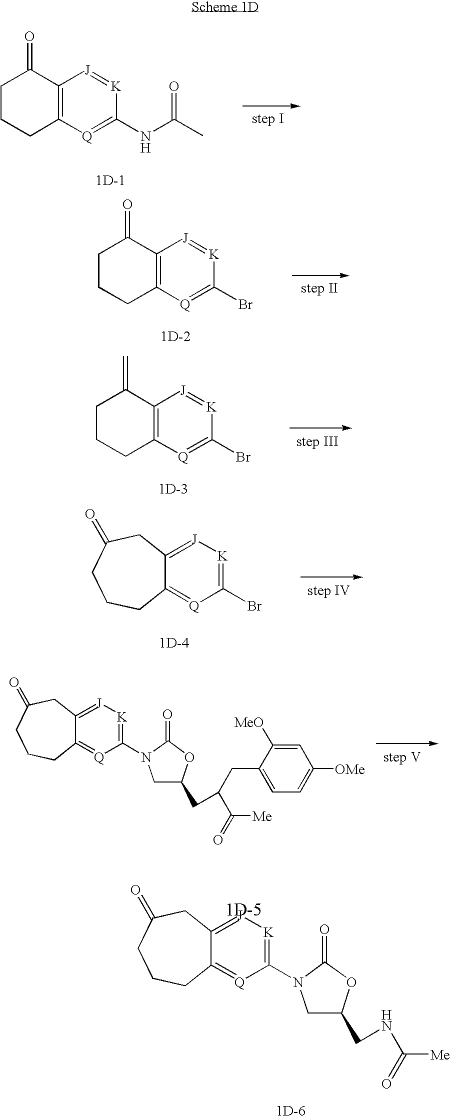

Schemes 2 A-C provide alternative approaches to the attachment of the oxazolidinone subunit of the invention compounds to the fused bicyclo ketone subunit. Method A commences with bromination of 2A-1 to provide 2A-2 (step I), followed by reduction of the ketone moiety (step II) to provide alocohol 2A-3. The alcohol moiety in 2A-3 is removed by techniques known to the skilled artisan (step III), for instance, via conversion to a leaving group such as a mesylate or tosylate or the like, followed by reduction using a trialkyl tin hydride, to provide bromide 2A-4. A variety of coupling procedures may be used to couple bromide 2A-4 to the requisite N-protected acetamide 2-4a (step 1V) to provide the protected core 2A-5. Deprotection and oxidation provides the target compound.

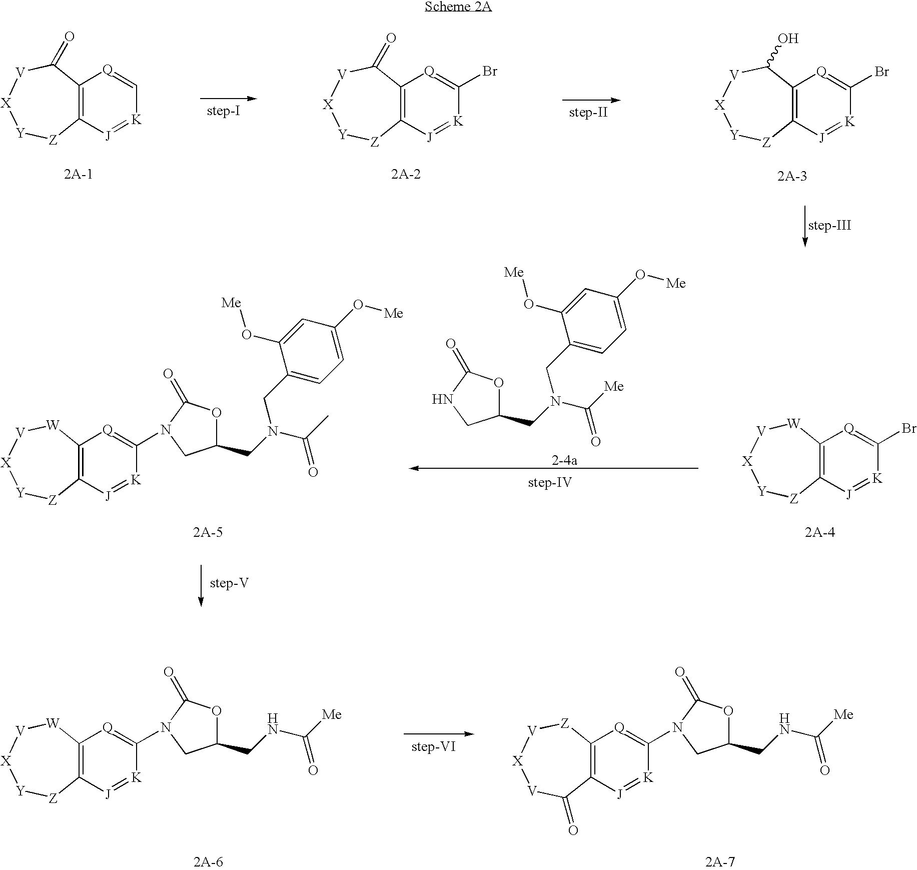

Method B of Scheme 2 provides another variant of the general approach. Thus, iodonitro compound 2B-1-1 is combined with methyl 4-pentynoate 2B-1-2 under conditions known to the skilled artisan (step 1) to provide the coupled product 2B-2. Reduction of the triple bond and nitro groups in 2B-2 (step II) provides methyl ester 2B-3. Acetylation of the amine moiety in 2B-3 (step III) and saponification of the methyl ester (step iv) yiels the acid 2B-5. Intramolecular cyclization of 2B-5 (step V), followed by elaboration of the oxazolidinone subunit (steps VI-X) provides the compound material 2B-11.

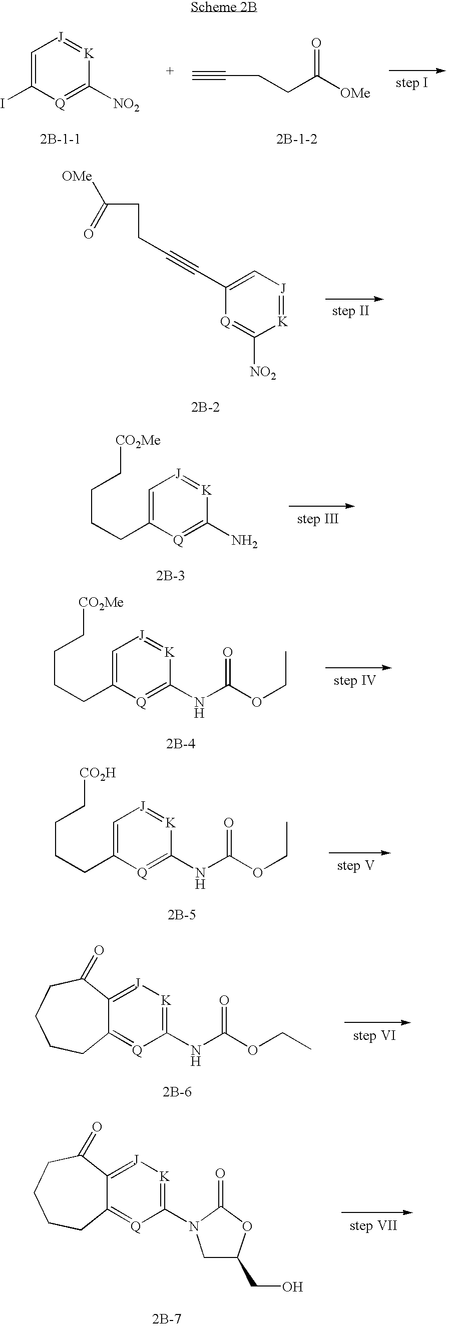

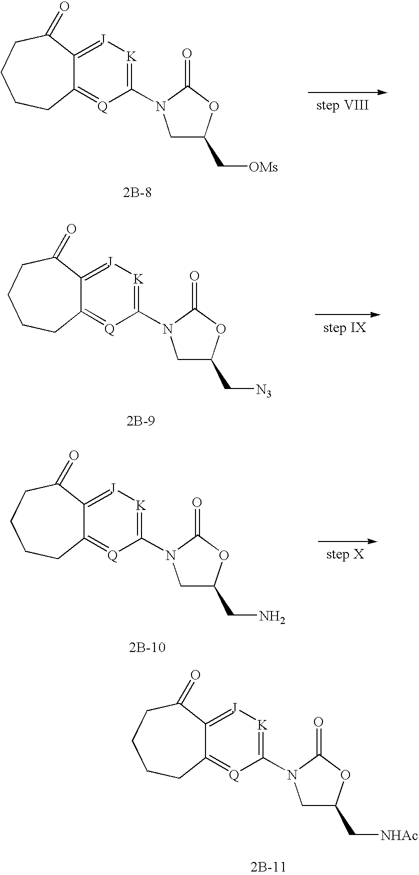

Scheme 2C provides an alternative strategy for the elaboration of the oxazolidinone subunit, compared to steps VI-X in Scheme 2B. Thus compound 2B-6 is treated with N-oxiranyl acetamide in the presence of base to provide 2-11.

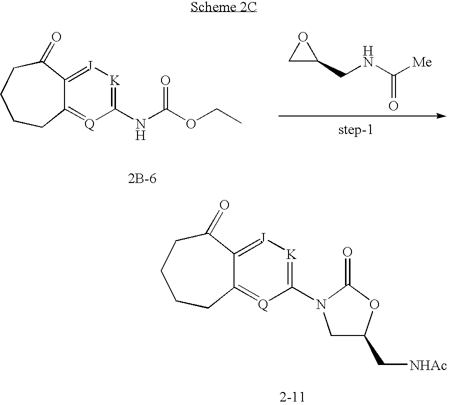

Schemes 3A and 3B provide an approach to unsaturated bicyclo saturated subunits. Thus, in Scheme 3A, reduction of the ketone moiety in 2B-7 (step 1), followed by conversion of the resulting alcohol moiety to a leaving group, and base mediated elimination (step II), provides the target compound 3A-2.

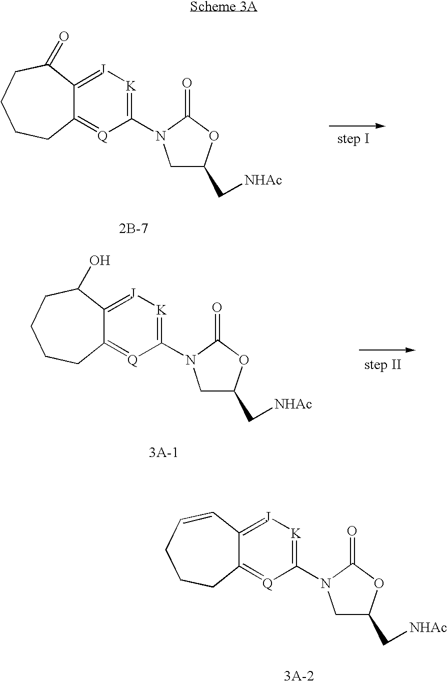

In Scheme 3B, ketone 1-9 is reduced (step 1) to provide alcohol 3B-1. Conversion of the alcohol moiety in 3B-1 to leaving group such as a mesylate or tosylate or the like, followed by base-mediated elimination (step II) provides the target compound 3B-2.

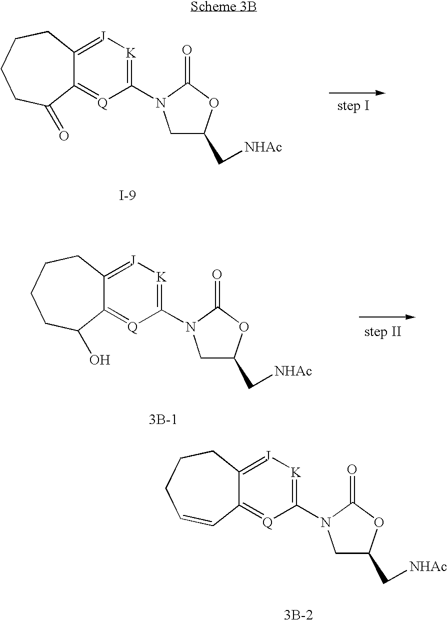

B. Compounds with Heteroatom-Containing Benzocycloheptyl Cores

Schemes 4-10 provide approaches to heteroatom containing bicyclo subunits. In particular, Scheme 4-5 provide approaches to sulfur-containing systems. Thus, ethyl 4-bromo-butanoate is coupled with bromo thiol 4-1 (step 1) to provide thioether 4-2. Following the approach outlined in Scheme 2 Method B for the conversion of 2-5B to 2-11, saponification of 4-2, followed by cyclization and elaboration of the oxazolidinone subunit provides the target compound 2-6.

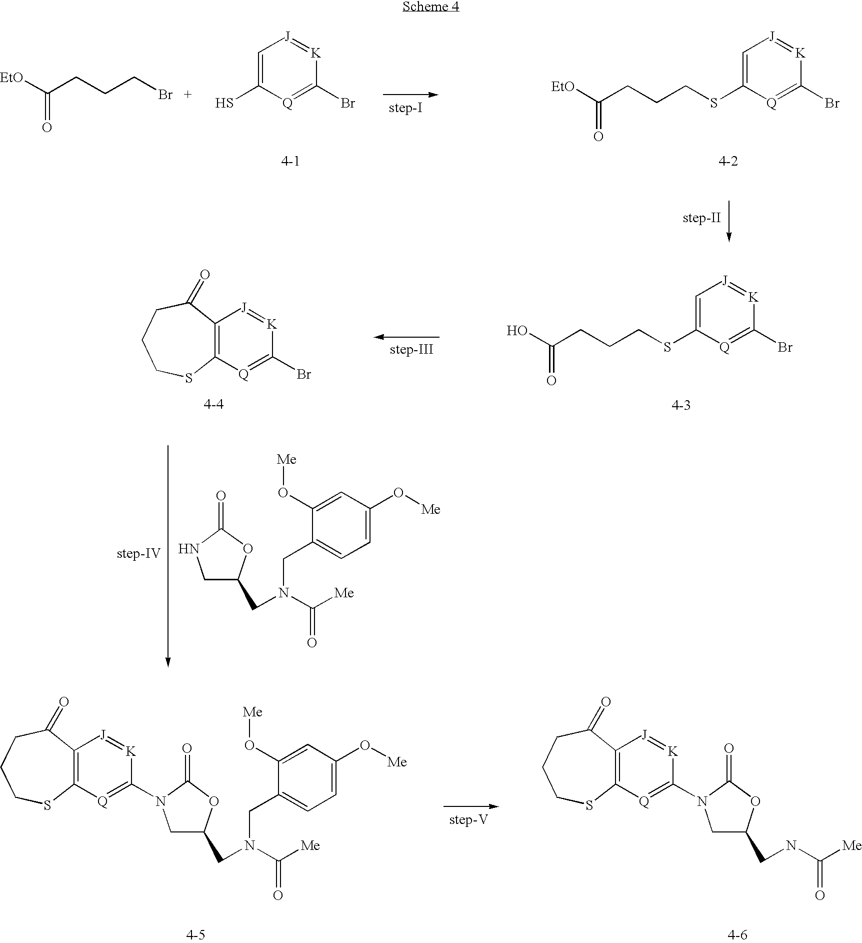

Scheme 5 provides a route for the preparation of sulfone-containing bicyclo oxazolidinone cores from the corresponding thioethers (e.g., compound 4-5). Thus, oxidation of the thioether moiety in 2-6 (step 1), followed by deprotection (step 2), provides sulfone 5-2.

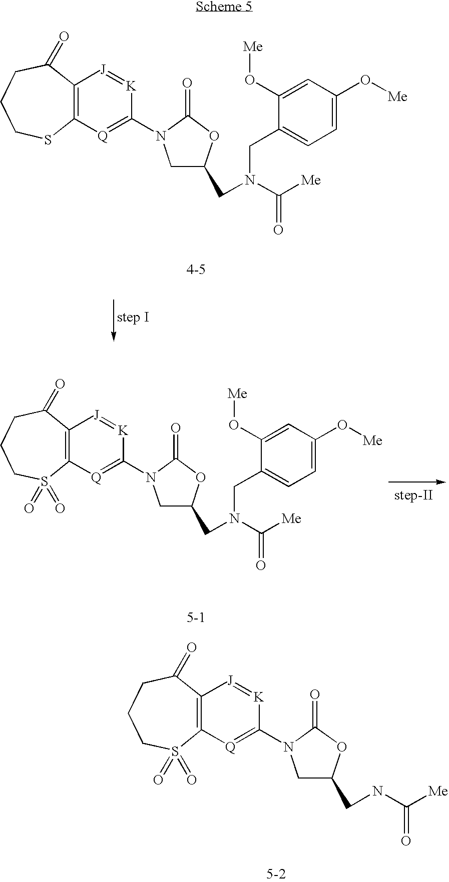

Schemes 6-8 provide approaches to oxygen-containing systems. Thus, ethyl 4-bromo-butanoate is coupled with bromo phenol 6-1 (step 1) to provide ether 4-2. Following the approach outlined in Scheme 2, Method B for the conversion of 2-5B to 2-11, saponification of compound 4-2, followed by cyclization and elaboration of the oxazolidinone subunit provides the target compound 2-6.

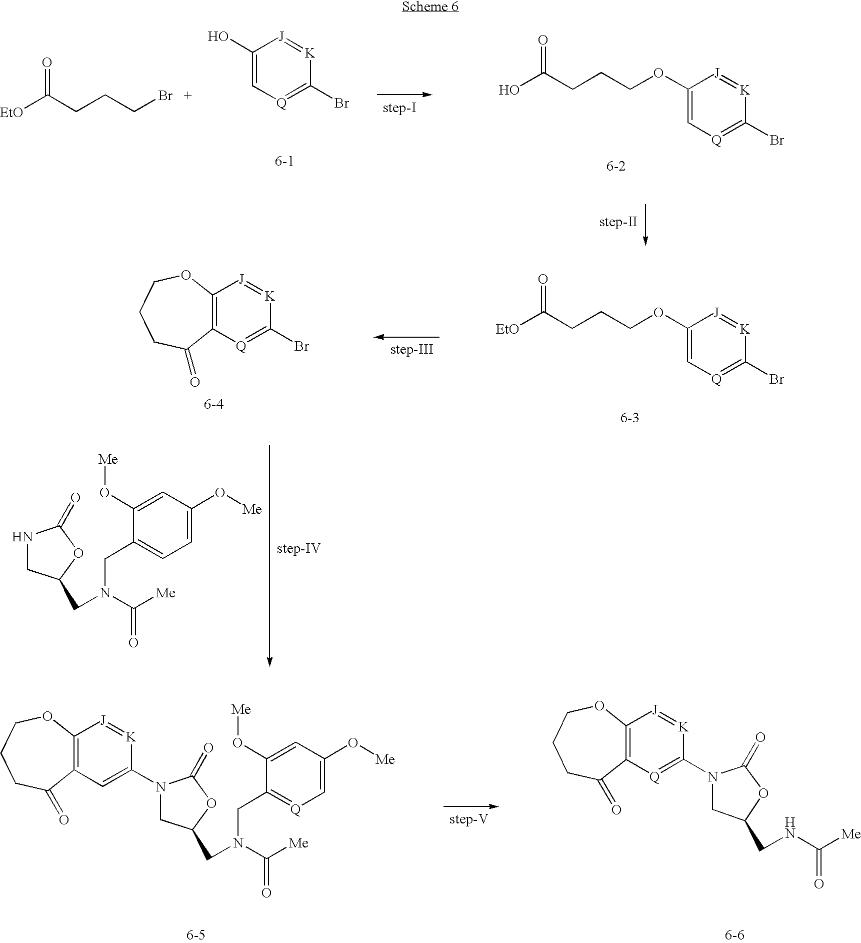

Scheme 7 depicts a variant of the Scheme 6 chemistry, wherein the ether linkage and ketone moiety are transposed in the target compound 7-6.

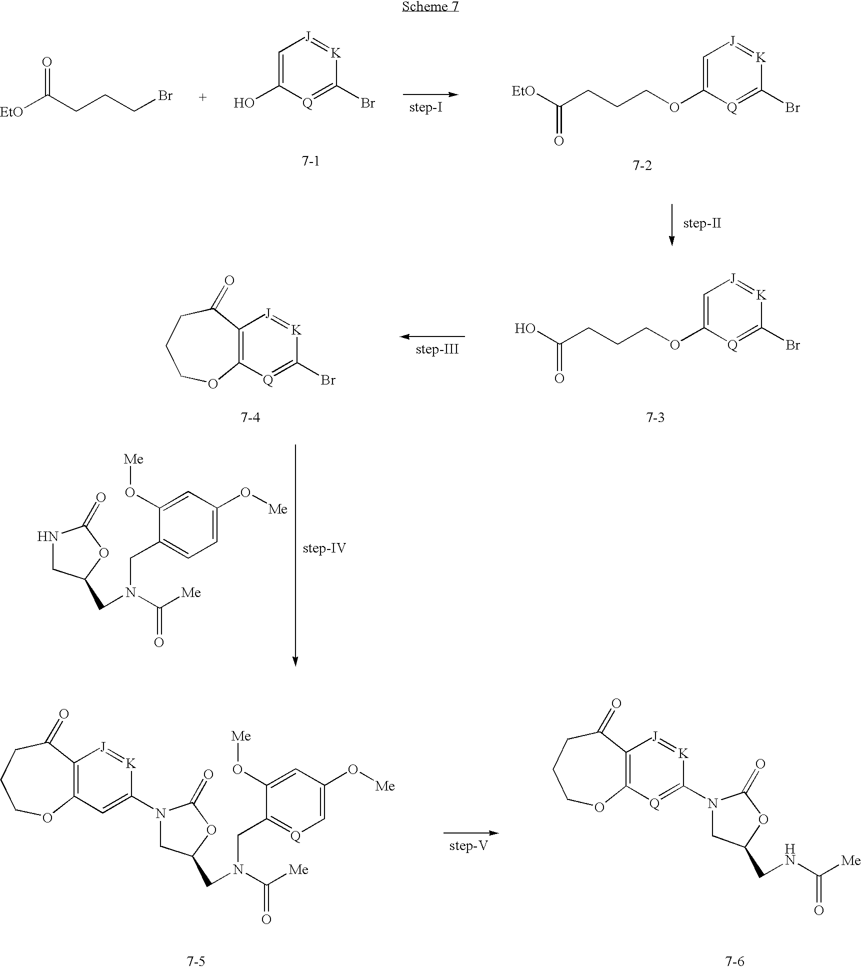

Schemes 8-11 provide approaches to oxygen-containing systems. Scheme 8 summarizes an approach to bicyclo subunits containing two heteroatoms (N and O). Thus, chroman-4-one analogue 8-1 undergoes bromination (step I) to provide 8-2. Hofmann-type ring enlargement of 8-2 (step II) provides amide 8-3, which is readily reduced (step IV) to give the amine 8-4. Protectino of the amine moiety in 8-4 (step V), followed by coupling of the oxazolidinone core (step VI) provides the intermediate 8-6. The acetamide side chain of the oxazolidinone is then elaborated (steps VII-IX), using the chemistry described for Scheme 2 Method B to provide the target compound 8-10.

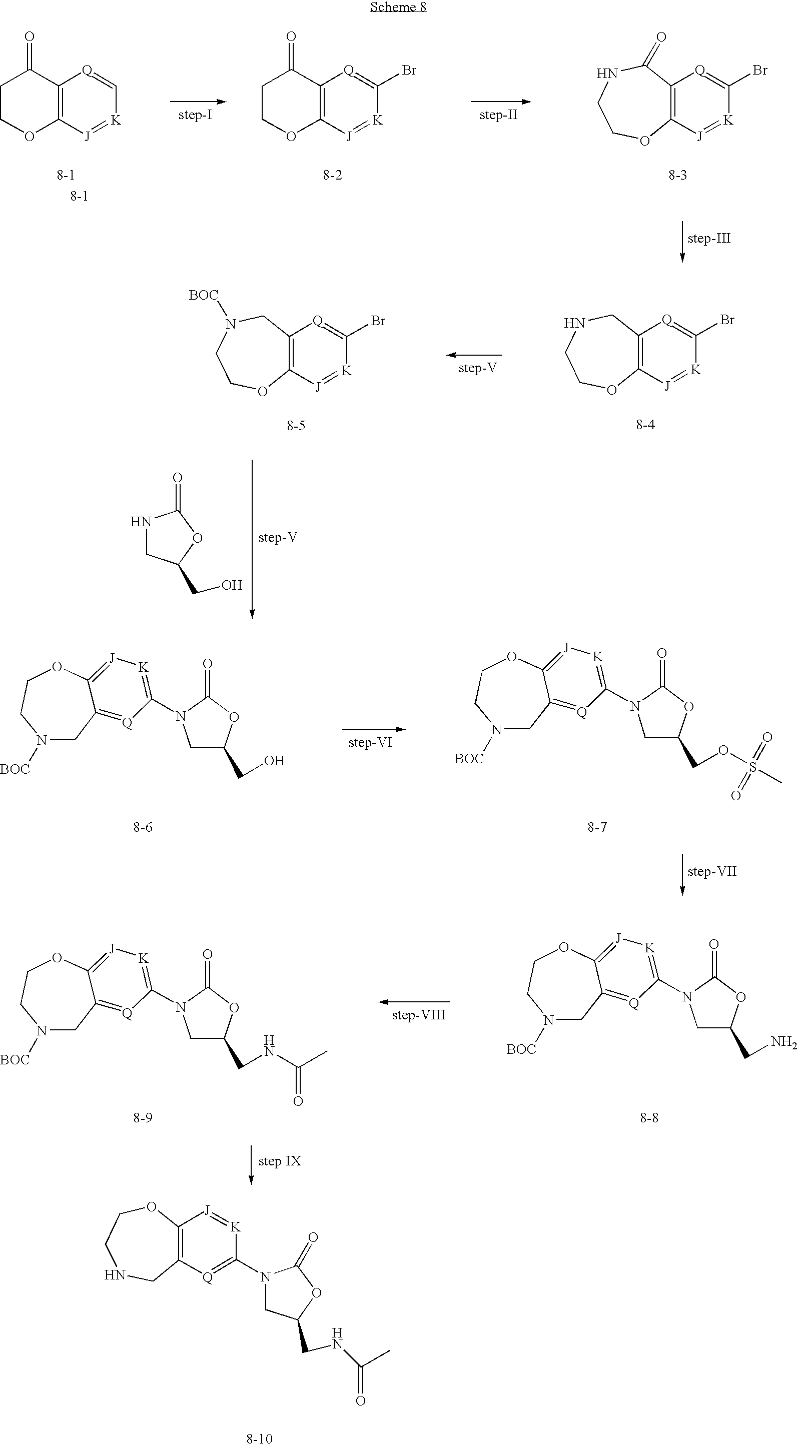

Schemes 9A and 9B provide a variant of the Scheme 8 approach that also affords bicyclo subunits containing an N linkage. Hofmann-type ring enlargement of from chroman-4-one analogue 9-1A (step I) provides amide 9-2A. Nitration (step II), and sequential reduction of the amide (step III), and nitro moieties (step IV) affords amine 9-5A. Protection of the amine moieties in 9-5A (step V), followed by coupling to the oxazolidinone core following the chemistry described above for Scheme I (step VI) provides the intermediate 9-6A. The acetamide side chain of the oxazolidinone is then elaborated (steps VII-X), using the chemistry described for Scheme 2 Method B to provide compound 9-11A, which is deprotected (step XI) to provide the target compound 9-12A.

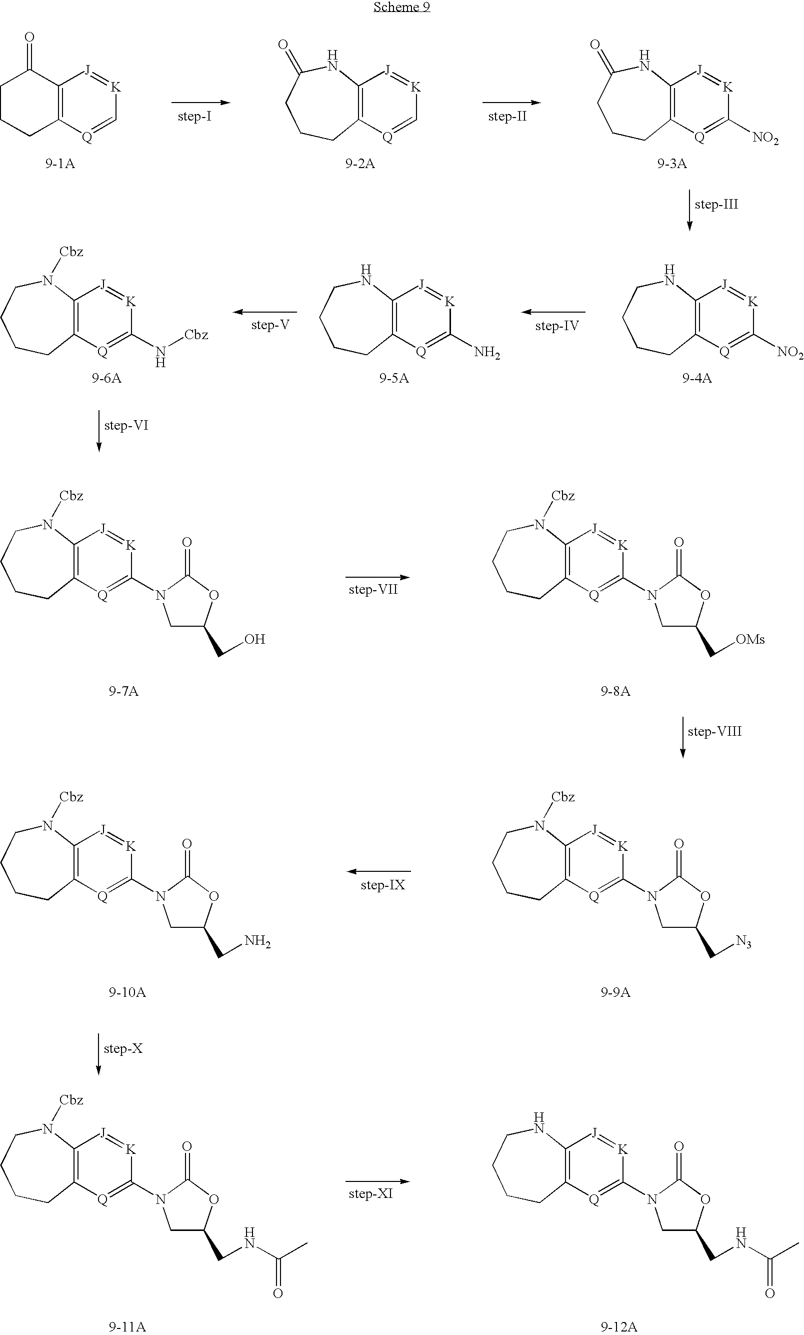

Scheme 9B provides an approach to amide 9B-3 in a minimum of steps. Thus, the brominated analogue is coupled to the oxazolidinone core (step I) as outlined in Scheme 2 Method A. Deprotection (step II) provides the target compound 9B-3.

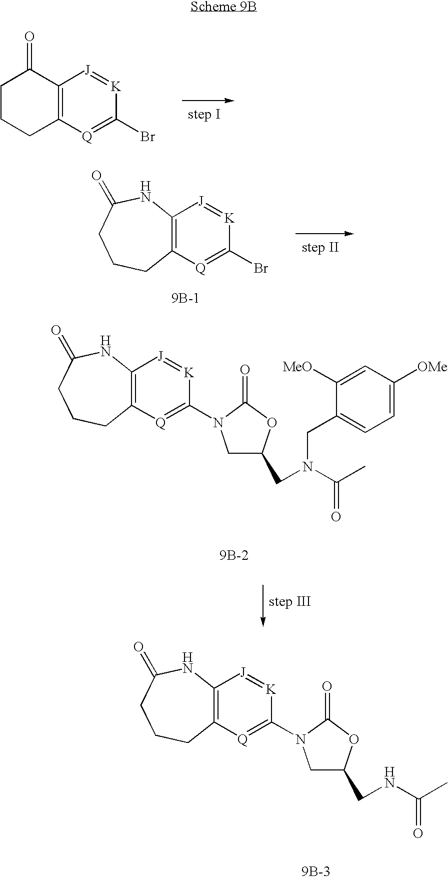

Scheme 10 provides approaches to bicyclo subunits containing N-linkages, wherein the N is “walked” around the ring. Hofmann-type ring enlargement of chromanone analogue 10-1 (step I) provides a mixture of amide products 10-2A and 10-2B. Upon separation, 10-2A and 10-2B are converted to target compounds 10-10A and 10-10B via a multistep sequence commencing with reduction of the amide moiety (step II); protection (step III); and attachment of the oxazolidinone subunit (step IV) to provide compounds 10-SA and 10-5B. The acetamide side chain of the oxazolidinone subunit is then elaborated (steps V/V′-VIII/VIII′), using the chemistry described for Scheme 2 Method B to provide the target compounds—10-9A and 10-9B, which are deprotected (step IX) to provide the target compound 10-10A and 10-10B.

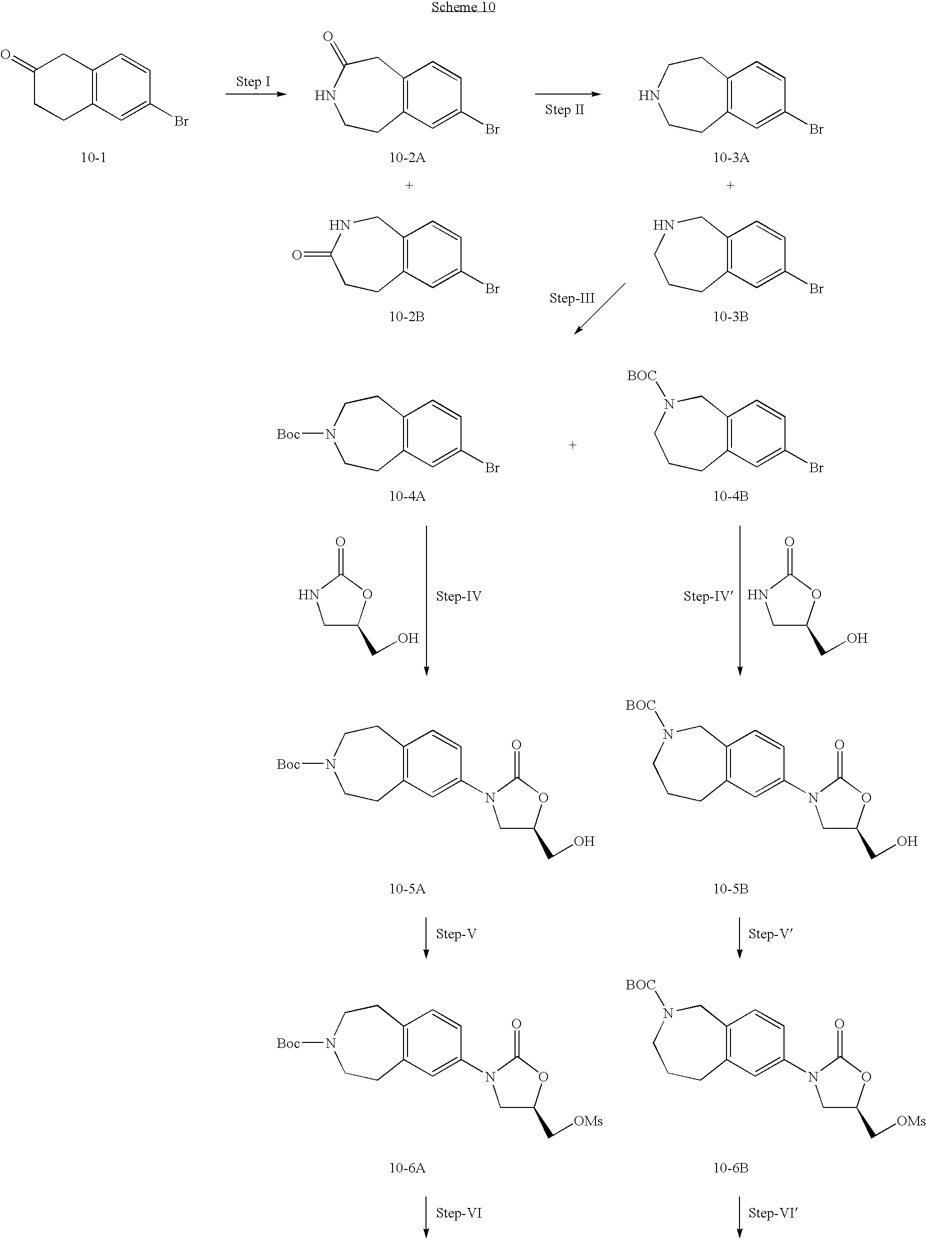

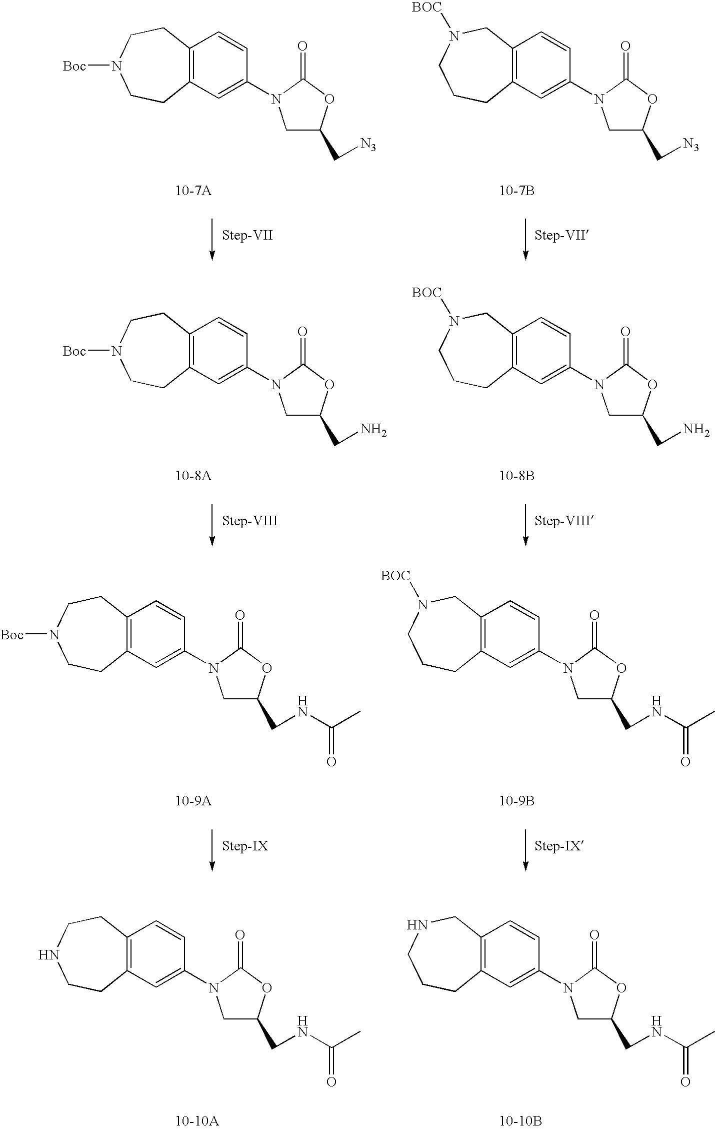

Pharmaceutical Formulations

The present invention also provides pharmaceutical compositions which comprise a bioactive invention compound or a salt such as a pharmaceutically acceptable salt thereof and optionally a pharmaceutically acceptable carrier. The compositions include those in a form adapted for oral, topical or parenteral use and can be used for the treatment of bacterial infection in mammals including humans.

The compounds, such as antibiotic compounds, also referred to herein as antimicrobial compounds, according to the invention can be formulated for administration in any convenient way for use in human or veterinary medicine, by analogy with other bioactive agents such as antibiotics. Such methods are known in the art and are not described in detail herein.

The composition can be formulated for administration by any route known in the art, such as subdermal, by inhalation, oral, topical or parenteral. The compositions may be in any form known in the art, including but not limited to tablets, capsules, powders, granules, lozenges, creams or liquid preparations, such as oral or sterile parenteral solutions or suspensions.

The topical formulations of the present invention can be presented as, for instance, ointments, creams or lotions, eye ointments and eye or ear drops, impregnated dressings and aerosols, and may contain appropriate conventional additives such as preservatives, solvents to assist drug penetration and emollients in ointments and creams.

The formulations may also contain compatible conventional carriers, such as cream or ointment bases and ethanol or oleyl alcohol for lotions. Such carriers may be present, for example, from about 1% up to about 98% of the formulation. For example, they may form up to about 80% of the formulation.

Tablets and capsules for oral administration may be in unit dose presentation form, and may contain conventional excipients such as binding agents, for example syrup, acacia, gelatin, sorbitol, tragacanth, or polyvinylpyrollidone; fillers, for example lactose, sugar, maize-starch, calcium phosphate, sorbitol or glycine; tabletting lubricants, for example magnesium stearate, talc, polyethylene glycol or silica; disintegrants, for example potato starch; or acceptable wetting agents such as sodium lauryl sulphate. The tablets may be coated according to methods will known in normal pharmaceutical practice.

Oral liquid preparations may be in the form of, for example, aqueous or oily suspensions, solutions, emulsions, syrups or elixirs, or may be presented as a dry product for reconstitution with water or other suitable vehicle before use. Such liquid preparations may contain conventional additives, such as suspending agents, for example sorbitol, methyl cellulose, glucose syrup, gelatin, hydroxyethyl cellulose, carboxymethyl cellulose, aluminium stearate gel or hydrogenated edible fats, emulsifying agents, for example lecithin, sorbitan monooleate, or acacia; non-aqueous vehicles (which may include edible oils), for example almond oil, oily esters such as glycerine, propylene glycol, or ethyl alcohol; preservatives, for example methyl or propyl p-hydroxybenzoate or sorbic acid, and, if desired, conventional flavoring or coloring agents.

For parenteral administration, fluid unit dosage forms are prepared utilizing the compound and a sterile vehicle, water being preferred. The compound, depending on the vehicle and concentration used, can be either suspended or dissolved in the vehicle or other suitable solvent. In preparing solutions, the compound can be dissolved in water for injection and filter sterilized before filling into a suitable vial or ampoule and sealing. Advantageously, agents such as a local anesthetic preservative and buffering agents can be dissolved in the vehicle. To enhance the stability, the composition can be frozen after filling into the vial and the water removed under vacuum. The dry lyophilized powder is then sealed in the vial and an accompanying vial of water for injection may be supplied to reconstitute the liquid prior to use. Parenteral suspensions are prepared in substantially the same manner except that the compound is suspended in the vehicle instead of being dissolved and sterilization cannot be accomplished by filtration. The compound can be sterilized by exposure to ethylene oxide before suspending in the sterile vehicle. Advantageously, a surfactant or wetting agent is included in the composition to facilitate uniform distribution of the compound.

The compositions may contain, for example, from about 0.1% by weight, e.g., from about 10-60% by weight, of the active material, depending on the method of administration. Where the compositions comprise dosage units, each unit will contain, for example, from about 50-500 mg of the active ingredient. The dosage as employed for adult human treatment will range, for example, from about 100 to 3000 mg per day, for instance 1500 mg per day depending on the route and frequency of administration. Such a dosage corresponds to about 1.5 to 50 mg/kg per day. Suitably the dosage is, for example, from about 5 to 20 mg/kg per day.

Biological Activity

The invention compounds can be screened to identify bioactive molecules with different biological activities using methods available in the art. The bioactive molecules, for example, can possess activity against a cellular target, including but not limited to enzymes and receptors, or a microorganism. A target cellular ligand or microorganism is one that is known or believed to be of importance in the etiology or progression of a disease. Examples of disease states for which compounds can be screened for biological activity include, but are not limited to, inflammation, infection, hypertension, central nervous system disorders, and cardiovascular disorders.

In one embodiment, the invention provides methods of treating or preventing an infectious disorder in a subject, such as a human or other animal subject, are provided, by administering an effective amount of an invention compound as disclosed herein to the subject. In one embodiment, the compound is administered in a pharmaceutically acceptable form optionally in a pharmaceutically acceptable carrier. As used herein, an “infectious disorder” is any disorder characterized by the presence of a microbial infection, such as bacterial infections. Such infectious disorders include, for example central nervous system infections, external ear infections, infections of the middle ear, such as acute otitis media, infections of the cranial sinuses, eye infections, infections of the oral cavity, such as infections of the teeth, gums and mucosa, upper respiratory tract infections, lower respiratory tract infections, genitourinary infections, gastrointestinal infections, gynecological infections, septicemia, bone and joint infections, skin and skin structure infections, bacterial endocarditis, burns, antibacterial prophylaxis of surgery, and antibacterial prophylaxis in immunosuppressed patients, such as patients receiving cancer chemotherapy, or organ transplant patients. The compounds and compositions comprising the compounds can be administered by routes such as topically, locally or systemically. Systemic application includes any method of introducing the compound into the tissues of the body, e.g., intrathecal, epidural, intramuscular, transdermal, intravenous, intraperitoneal, subcutaneous, sublingual, rectal, and oral administration. The specific dosage of antimicrobial to be administered, as well as the duration of treatment, may be adjusted as needed.

The compounds of the invention may be used for the treatment or prevention of infectious disorders caused by a variety of bacterial organisms. Examples include Gram positive and Gram negative aerobic and anaerobic bacteria, including Staphylococci, for example S. aureus; Enterococci, for example E. faecalis; Streptococci, for example S. pneumoniae; Haemophilus, for example H. influenza; Moraxella, for example M. catarrhalis; and Escherichia, for example E. coli. Other examples include Mycobacteria, for example M. tuberculosis; intercellular microbes, for example Chlamydia and Rickettsiae; and Mycoplasma, for example M. pneumoniae.

The ability of a compound of the invention to inhibit bacterial growth, demonstrate in vivo activity, and enhanced pharmacokinetics are demonstrated using pharmacological models that are well known to the art, for example, using models such as the tests described below.

Test A—Antibacterial Assays

The compounds of the present invention were tested against an assortment of Gram-negative and Gram-positive organisms using standard microtitration techniques (Cohen et. al., Antimicrob., 1985;28:766; Heifetz, et. al., Antimicrob., 1974;6:124). The results of the evaluation are shown in Tables 1A and B.

TABLE 1A

| | Minimum Inhibitory Concentrations μg/mL | | |
|---|---|---|---|
| | Gram Negative Bacteria | | |
| | H. influenzae | M. catarrhalis | E. coli |
| Compound No. or Structure | H13542 | BC3534 | Tol C |
| 2 | 8 | 4 | >64 |
| 4 | 64 | 32 | >64 |
| 5 | 64 | 64 | >64 |
| 6 | >64 | 32 | >64 |
| 7 | 32 | 32 | >64 |
| 12 | 32 | 16 | >64 |
| 14 | 64 | >64 | >64 |
| 15 | >64 | >64 | >64 |
| 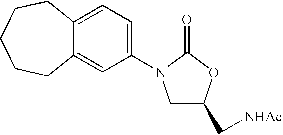 | >64 | 16 | >64 |
| 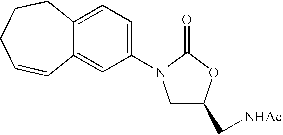 | >64 | 32 | >64 |
| 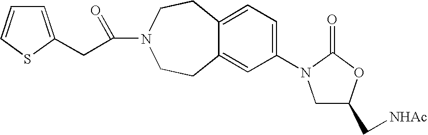 | >64 | 16 | >64 |
| 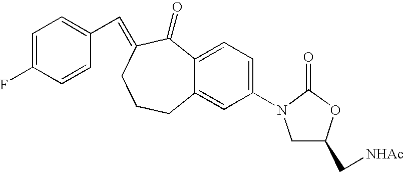 | 2 | 1 | >64 |
| 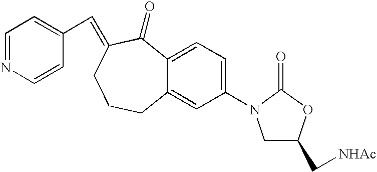 | 16 | 8 | >64 |
| 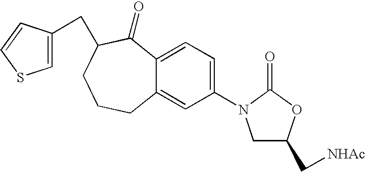 | 2 | 1 | >64 |
| 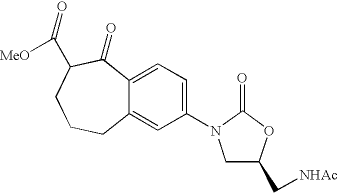 | 32 | 16 | >64 |

TABLE 1B

| | Minimum Inhibitory Concentrations μg/mL | | |
|---|---|---|---|
| | Gram Positive Bacteria | | |
| Compound Structure or | E. faecalis | S. aureus | S pyogenes |
| Example No. | MGH-2 | UC-76 | C203 |
| 2 | >64 | >64 | >64 |
| 4 | 8 | 4 | 4 |
| 5 | 16 | 8 | 8 |
| 6 | 4 | 4 | 2 |
| 7 | 4 | 4 | 2 |
| 12 | 4 | 8 | 2 |
| 14 | 64 | 32 | 16 |
| 15 | 64 | 64 | 64 |
| 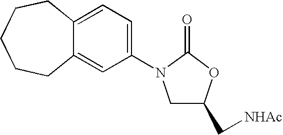 | 4 | 2 | 2 |
| 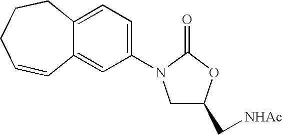 | 4 | 4 | 2 |
| 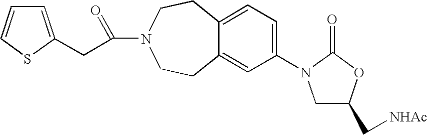 | 0.25 | 0.25 | 0.125 |
| 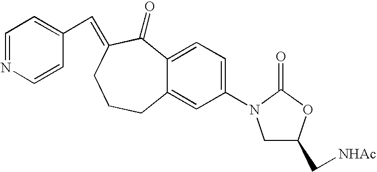 | 1 | 1 | 1 |
| 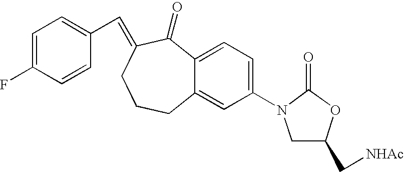 | 0.5 | 0.5 | 0.5 |
| 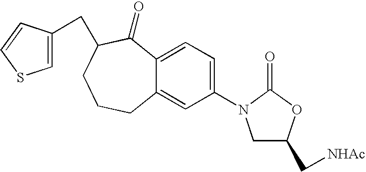 | 0.5 | 0.25 | 0.25 |
| 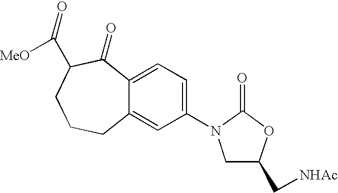 | 2 | 4 | 4 |

The compounds of the present invention were tested against E. coli transcription and translation (TnT) assay. The TnT assay is a cell free system that utilizes an E. coli S30 fraction and a “premix” to transcribe and translate the firefly luciferase gene from an exogenously supplied plasmid DNA. The amount of luciferase produced is measured by observing the luminescence produced after addition of a luciferase assay reagent. The TnT assay reagents, including the luciferase reporter plasmid pBESTluc, were purchased from Promega Corporation. The protocol was based upon the manufacturer's instructions (Promega Technical Bulletin number 92 “E. coli S30 Extract System for Circular DNA”). Luciferase assay reagent (LucLite Plus) was purchased from Packard Biosciences.

The assay was conducted in white, flat-bottomed, polystyrene 96-well plates. Each well contained S30, premix, amino acids, compound and DNA in a total volume of 35 microliters. The reactions were allowed to incubate at room temperature for 20 minutes, then quenched with 35 microliters of LucLite Plus. The plate was then sealed with an aluminum foil lid and allowed to mix on a plate shaker for five minutes. The plate was then uncovered and read on the LJL Analyst using the standard luminescence protocol. The assay can also be read with a Perkin-Elmer Microbeta Trilux using a 1450-105 96 well plate cassette utilizing a protocol with a 10 second counting time, no background correction, and upper PMT usage. The results of the evaluation are shown in Table 1C.

TABLE 2C

| Compound Structure or | Minimum Inhibitory Concentrations μg/mL |
|---|---|
| Example No. | E. coli TnT Assay |
| 2 | 3.9 |
| 4 | 8.2 |
| 5 | 2.3 |
| 6 | 15 |
| 7 | 12 |
| 12 | 1.1 |
| 14 | No data |
| 15 | 11 |
| 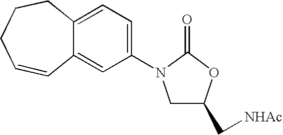 | 15 |
| 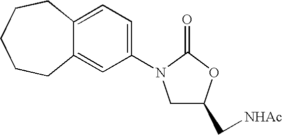 | 6.6 |
| 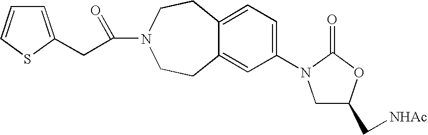 | 2.3 |
| 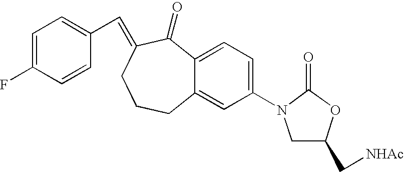 | 1.4 |
| 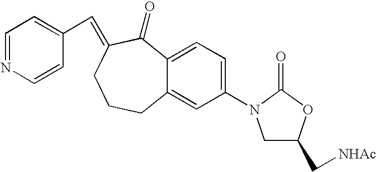 | 2.7 |
| 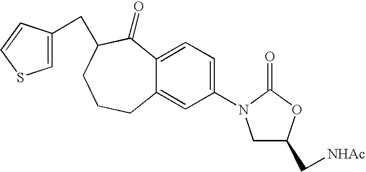 | 0.84 |
| 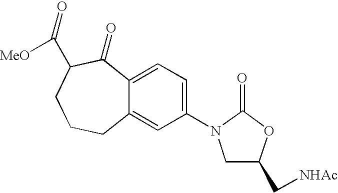 | 4.7 |

The following examples are provided to illustrate but not limit the claimed invention.

### EXAMPLE 1

### N-[2-oxo-3-(9-oxo-6, 7, 8, 9-tetrahydro-5H-benzocyclohepten-2-yl)-oxazolidin-5-yl-methyl]acetamide (I-9)

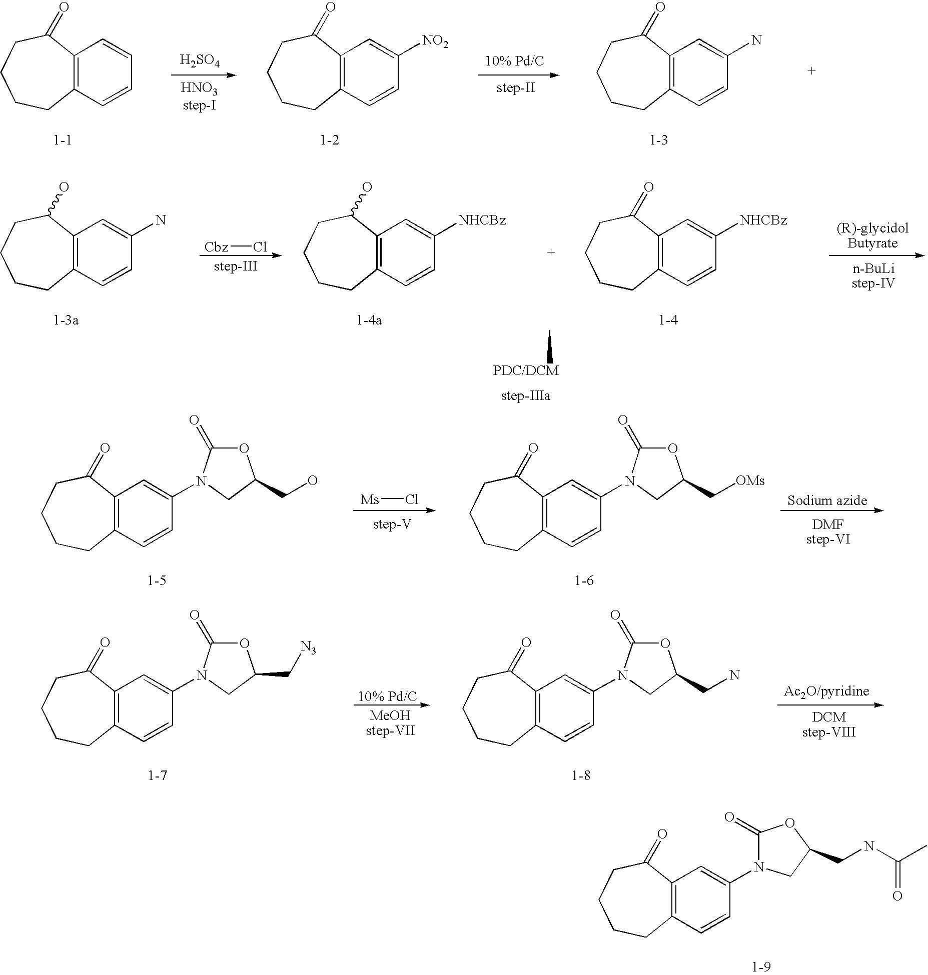

A. Preparation of 3-nitro-6, 7, 8, 9-tetrahydro-benzocyclohepten-5-one (Scheme I, Step I, I-2):

1-Benzosuberone (175.0 g, 1.09 mol) was dissolved in concentrated sulfuric acid (3.5 L) and cooled to 0° C. To this mixture was added drop wise, a solution of fuming nitric acid (65.03 ml, 1.13 mol) in sulfuric acid (425 ml) and after the addition was complete, the reaction mixture was stirred at 0° C. for 30 min. The reaction mixture was poured into ice water and extracted with diethyl ether (3×2.0 L). The organic extracts are pooled, washed with brine, dried over anhydrous Na₂SO₄, filtered and evaporated under vacuum. The residue obtained was triturated with hexane (3×1.0 L), filtered and dried to give the title compound. Yield: 125.0 g (55.8%), mp. 88-89° C.

B. Preparation of 3-amino-6, 7, 8, 9-tetrahydro-benzocycloheptene-5-one (Scheme I, Step II, I-3):

A solution of 3-nitro-6, 7, 8, 9-tetrahydro-benzocyclohepten-5-one (I-2) in methanol (1.5 L) was hydrogenated in presence of 10% Pd/C (15.2 g) at 50 psi for 1 h and filtered through Celite. The filtrate is evaporated under vacuum to give a solid (105 g). After concluding that, it is a mixture of desired amino compound and an over reduced product, (I-3a, 3-amino-5-hydroxy-6, 7, 8, 9-tetrahydro-benzocycloheptane) the mixture has been directly subjected to the next step, without further purification. Yield: 105 g (99%).

C. Preparation of (9-oxo-6, 7, 8, 9-tetrahydro-5H-benzocyclohepten-2-yl)carbamic acid benzyl ester (Scheme I, step III, I-4):

A solution of the mixture obtained from step-3 (3-amino-6, 7, 8, 9-tetrahydro-benzocyclohepten-5-one (I-3) and 3-amino-5-hydroxy-6, 7, 8, 9-tetrahydro-benzocycloheptane (I-3a, 105.0 g, 0.6 mol) in a mixture of acetone/water, 2:½L: 1 L) was added sodium bicarbonate (189.0 g, mol) and cooled to 0° C. The reaction mixture was treated with Cbz-chloride (189 ml, 1.32 mol), stirred over night at room temperature and the acetone is removed in vacuo. The aqueous residue is extracted with ethyl acetate (3×1.0 L) and the organic extracts are pooled, washed with brine, dried over anhydrous Na₂SO₄ and concentrated. The residue was purified by silica gel column chromatography (TLC-EtOAc:Hex/3:7) to give both the title compound along with [N-5-hydroxy(1, 2, 3, 4, 5-pentahydrobenzo[a][7]annulene-7-yl) (phenylmethoxy) carboxamide], I-4a. Yield of 4:39.0 g. Yield of 4a: 105.0 g

A solution of [N-5-hydroxy(1, 2, 3, 4, 5-pentahydrobenzo[a][7]annulene-7-yl)(phenylmethoxy)carboxamide] (I-4a) in DCM (1.5 L) was treated with PDC (120 g) and stirred over night at room temperature. The reaction mixture is filtered through a bed of Celite and the filtrate is evaporated under vacuum to give the title compound. Total Yield: 114.0 g (62%), mp. 120-121° C.

D. Preparation of 5-hydroxymethyl-3-(9-oxo-6, 7, 8, 9-tetrahydro-5H-benzocyclohepten-2-yl)-oxazolidin-2-one (Scheme I, step IV, I-5):

To a flame-dried flask charged with diisopropyl amine (19.5 ml, 0.139 mol) and THF (400 ml) was added n-butyl lithium (68.55 ml, 2.5 M in heaxanes) drop wise at −78° C. The reaction mixture was allowed to warm to 0° C. and then transferred by a canula in to a separate flask containing (9-oxo-6, 7, 8, 9-tetrahydro-5H-benzocyclohepten-2-yl)carbamic acid benzyl ester (I-4, 39.0 g, 0.109 mol) in THF (800 ml) at −78° C. The reaction mixture was stirred at −78° C. for 30 min. and was added R-glycidol butyrate (19.5 g, 0.135 mol). The reaction mixture was warmed to room temperature, then heated at 70° C. for 12 h and quenched by diluting with saturated solution of ammonium chloride (500 ml). The aqueous mixture was extracted with ethyl acetate (3×1 L) and the combined organic fractions were washed with brine, dried over anhydrous Na₂SO₄, filtered and evaporated under vacuum. The residue obtained was triturated with ether to give the title compound, which was used in the next step without further purification. Yield: 34.2 g, mp. 144-146° C.

E. Preparation of methanesulfonic acid -2-oxo-3-(9-oxo-6, 7, 8, 9-tetrahydro-5H-benzocyclohepten-2-yl)oxazolidin-5-yl methyl ester (Scheme I, step V, I-6):

To a solution of 5-hydroxymethyl-3-(9-oxo-6, 7, 8, 9-tetrahydro-5H-benzocyclohepten-2-yl)-oxazolidin-2-one (I-5, 34.2 g, 0.130 mol) in methylene chloride (1.0 L), at 0° C. was added triethyl amine (36.8 ml, mol) followed by methane sulfonyl chloride (13.5 ml, 0.174 mol). The reaction mixture was let to warm to room temperature, stirred for 2 h, diluted with ethyl acetate. The ethyl acetate solution was washed with brine (3×300 ml), dried over anhydrous Na₂SO₄, filtered and evaporated under vacuum to give the title compound. Yield: 42.0 g (93%).

F. Preparation of 5-azidomethyl-3-(9-oxo-6, 7, 8, 9-tetrahydro-5H-benzocyclohepten-2-yl)oxazolidin-2-one (Scheme I, step VI, I-7):

To a solution of methanesulfonic acid-2-oxo-3-(9-oxo-6, 7, 8, 9-tetrahydro-5H-benzocyclohepten-2-yl)oxazolidin-5-yl methyl ester (I-6, 42.0 g, 0.123 mol) in DMF (300 ml) was added sodium azide (29.4 g, 0.452 mol, and heated over night at 70° C. The reaction mixture was diluted with ethyl acetate (1.0 L), washed with water (3×300 ml), brine (1×500 ml), dried over anhydrous Na₂SO₄, filtered and evaporated under vacuum to give the title compound, which was directly used in the next step. Yield: 34.5 g (93%).

G. Preparation of 5-aminomethyl-3-(9-oxo-6, 8, 9-tetrahydro-5H-benzocyclohepten-2-yl)-oxazolidin-2-one (Scheme I, step VII, I-8):

A solution of 5-azidomethyl-3-(9-oxo-6, 7, 8, 9-tetrahydro-5H-benzocyclo-hepten-2-yl)oxazolidin-2-one (I-7, 34.5 g, 0.115 mol) in methanol (1.0 L) was hydrogenated in presence of 10% Pd/C (11.71 g) at 35 psi for 1 h and filtered through a short bed of celite. Evaporation of the filtrate under vacuum gave the title compound, which was used in the proceeding step without further purification. Yield: 27.4 g (85%)

H. Preparation of N-[2-oxo-3-(9-oxo-6, 7, 8, 9-tetrahydro-5H-benzocyclohepten-2-yl)-oxazolidin-5-yl-methyl]acetamide (Scheme I, step VIII, I-9):

To a flame dried flask was charged 5-aminomethyl-3-(9-oxo-6, 8, 9-tetrahydro-5H-benzocyclohepten-2-yl)-oxazolidin-2-one (1-8, 27.4 g, 0.1 mol) and pyridine (25.0 ml, mol) in methylene chloride (1.0 L) at 0° C., then was added acetic anhydride (13.12 ml). The reaction mixture allowed to come to room temperature and stirred over night and evaporated under vacuum. The residue obtained was purified by silica gel column chromatography (50% EtOAc in hexanes to 100% EtOAc) to give the title compound. Yield: 12.5 g (39.5%), mp. 122-123° C.

### EXAMPLE 2

### 5 N-[2-Oxo-3-(5-oxo-6,7,8,9-tetrahydro-5H-benzocyclohepten-2-yl)-oxazolidin-5-ylmethyl]-acetamide (II-7)

Three methods are provided for the synthesis of the title compound.

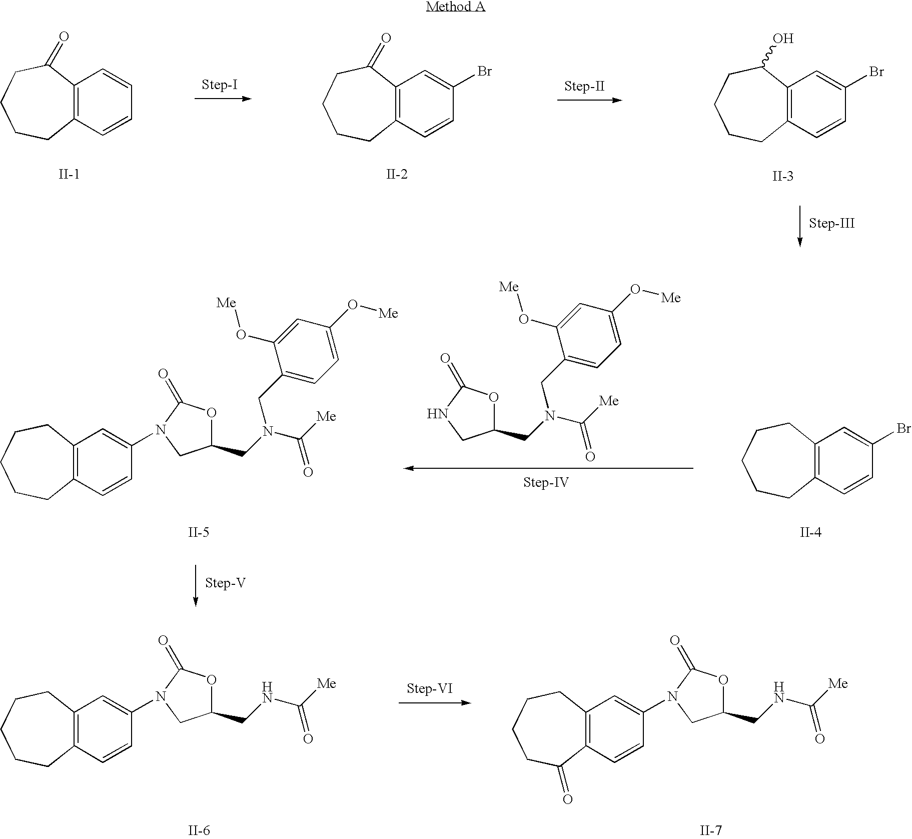

A. 3-Bromo-6,7,8,9-tetrahydro-benzocyclohepten-5-one (Step-I, II-2)

To the vigorously stirred aluminum chloride was added 1-benzosuberone. After 10 minutes, bromine was added slowly and the reaction mixture was stirred at room temperature for 5 minutes and then heated to 80° C. for 5 minutes. The reaction was then quenched by slowly adding a mixture of crushed ice and 1N hydrochloric acid. After the addition, the reaction mixture was slowly cooled to room temperature and ether was added. The resulting mixture was stirred until the residue had dissolved. The layers were then separated and the aqueous layer extracted once with diethyl ether. The combined organic extracts were washed with brine, dried over sodium sulfate and concentrated to a residue, which was purified by column chromatography. (21% yield). H¹NMR (400 MHz, CDCl₃): δ 1.83 (m, 4H), 2.71 (apparent triplet, 2H), 2.87 (apparent triplet, 1H), 7.07 (d, J=7.9 Hz, 1 H), 7.51 (d,d, J=8.3, 2.5 Hz, 1 H), 7.82, (d, J=2.8 Hz, 1H).

B. 3-Bromo-6,7,8,9-tetrahydro-5H-benzocyclohepten-5-ol (Step-II, II-3)

To a solution of 3-Bromo-6,7,8,9-tetrahydro-5H-benzocyclohepten-5-ol (II-2, 1.01 g, 4.22 mmol) in dichloromethane (21 mL) cooled to 0° C. was added sodium borohydride (175 mg, 4.64 mmol). The ice bath was removed and the reaction stirred one hour. No product was observed by LCMS so DMF (5.0 mL) and methanol (5.0 mL) was added followed by an additional 1.5 equivalents of sodium borohydride (239 mg, 6.33 mmol). The reaction was stirred at room temperature over night and was then diluted with ethyl acetate and washed with water 4×, brine, dried over sodium sulfate and concentrated. Chromatographed on an Isco 10 g column eluting with 0-20% ethyl acetate in hexanes over 30 minutes gave the title compound as a white solid (880 mg, 86%). H¹NMR (400 MHz, CDCl₃): δ 1.77 (m, 4H), 1.99 (m, 2H), 2.64 (m, 1H), 2.81 (m, 1H), 4.88 (d, J=9.6 Hz, 1H), 6.94 (d, J=7.9 Hz, 1H), 7.25 (d,d, J=7.9, 2.1 Hz, 1H), 7.61 (d, J=1.7 Hz, 1H). MS (CI) 223.1 (M−17 (loss of OH)).

C. 2-Bromo-6,7,8,9-tetrahydro-5H-benzocycloheptene (Step-III, III-4)

To a solution of 3-Bromo-6,7,8,9-tetrahydro-5H-benzocyclohepten-5-ol (1-3, 520 mg, 2.16 mmol) in dichloromethane (7.3 mL) was added triethylsilane (0.63 mL, 3.94 mmol) followed by drop wise addition of trifluoroacetic acid (1.56 m/L, 20.2 mmol). The reaction stirred at room temperature over night. The solvent was removed on the rotary evaporator and the resulting residue was dissolved in ethyl acetate and a saturated solution of sodium bicarbonate. The mixture was stirred vigorously for several minutes and then the layers were separated. The organic layer was washed twice with sat. sodium bicarbonate, brine, dried over sodium sulfate, and concentrated. Chromatographed on a Biotage Flash 40S column eluting with 100% hexanes gave the title compound (309 mg, 63% Yield). H¹NMR (400 MHz, CDCl₃): δ 1.61 (m, 4H), 1.81 (m, 2H), 2.71 (m, 4H), 6.94 (d, J=7.9 Hz, 1H), 7.17 (d,d, J=7.9, 2.1 Hz, 1H), 7.22 (d, J=2.1 Hz, 1H).

D. N-(2,4-Dimethoxy-benzyl)-N-[2-oxo-3-(6,7,8,9-tetrahydro-5H-benzocyclohepten-2-yl)-oxazolidin-5-ylmethyl]-acetaniide (Step-IV, III-5)

The title compound was prepared from 2-bromo-6,7,8,9-tetrahydro-5H-benzocycloheptene (II-4) and N-(2,4-dimethoxy-benzyl)-N-(2-oxo-oxazolidin-5-ylmethyl)-acetamide according to procedure as described in Scheme IV, step IV (Yield: 260 mg, 42%). MS (CI) m/z 453.4 (M+1), 497.4 (M−1+46).

E. N-[2-Oxo-3-(6,7,8,9-tetrahydro-5H-benzocyclohepten-2-yl)-oxazolidin-5-ylmethyl]-acetamide (Step-V, III-6)

The title compound was prepared from N-(2,4-dimethoxy-benzyl)-N-[2-oxo-3-(6,7,8,9-tetrahydro-5H-benzocyclohepten-2-yl)-oxazolidin-5-ylmethyl-acetamide (II-5) according to procedure as described in Scheme IV, step V (Yield: 124 mg, 71%). H¹NMR (400 MHz, CDCl₃): δ 1.61 (m, 4H), 1.81 (m, 2H), 2.76 (m, 4H), 2.75 (m, 2H), 3.58 (dt, J=14.5, 6.2 Hz, 1H), 3.74 (m, 2H), 4.03 (t, J=9.1 Hz, 1H), 4.74 (m, 1H), 5.99 (broad singlet, 1H), 7.07 (d, J=8.3 Hz, 1H), 7.17 (dd, J=7.9, 2.5 Hz, 1H), 7.25 (s, 1H). MS (CI) m/z 303.3 (M+1).

F. N-[2-Oxo-3-(5-oxo-6,7,8,9-tetrahydro-5H-benzocyclohepten-2-yl)-oxazolidin-5-ylmethyl]-acetamide (Step-VI, II-7)

To a solution of N-[2-Oxo-3-(6,7,8,9-tetrahydro-5H-benzocyclohepten-2-yl)-oxazolidin-5-ylmethyl]-acetamide (II-6, 50 mg, 0.165 mmol) in acetic acid (0.5 mL) and acetic anhydride (0.068 mL) was added a solution of chromium trioxide (67 mg, 0.23 mmol) in acetic acid (0.3 mL) and water (0.063 mL). The reaction stirred at room temperature over night followed by addition of more chromium trioxide (30 mg, 0.30 mmol). The reaction was stirred over night and additional water (0.06 mL) was added. The reaction stirred open to the air for three hours and was then diluted with water and extracted with twice with EtOAc. The combined organic layers were washed with brine, dried over sodium sulfate and concentrated in vacuo. Purification using silica gel chromatography gave the title compound (7.5 mg, 14% Yield). H¹NMR (400 MHz, CDCl₃): □ 1.79 (m, 2H), 1.85 (m, 2H), 2.01 (s, 3H), 2.71 (m, 2H), 2.93 (m, 2H) 3.64 (m, 1H), 3.69 (m, 1H), 4.08 (m,1H), 4.78 (m, 1H), 6.15 (s, 1H), 7.39 (d, J=7.9 Hz, 1H), 7.45 (s, 1H), 7.77 (d, J=7.9 Hz, 1H). MS (CI) m/z 317.3 (M+1).

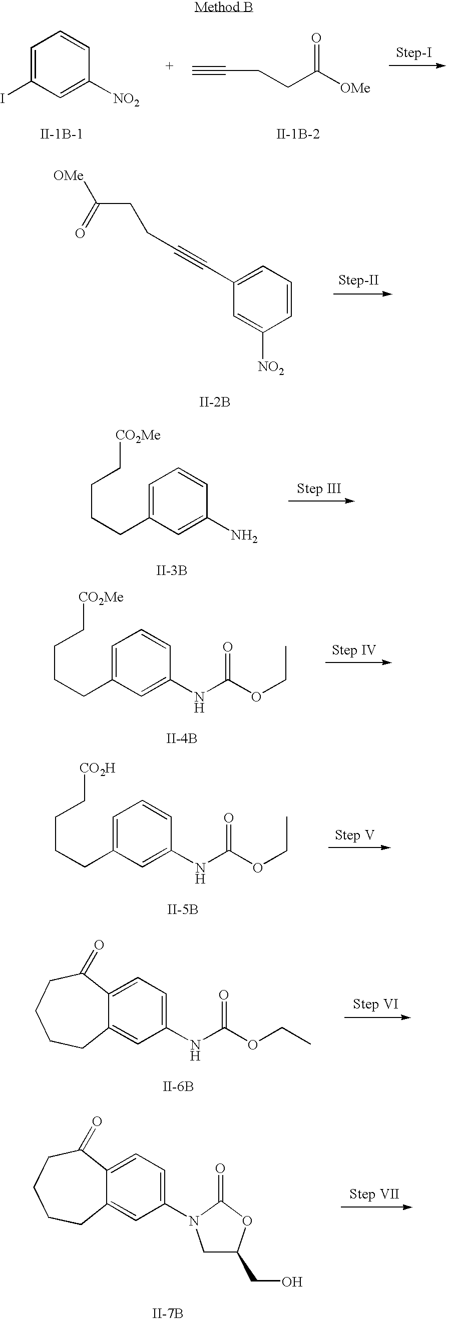

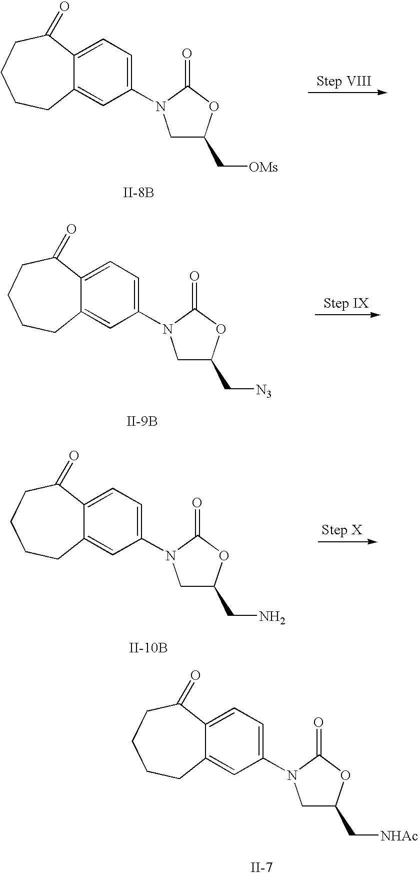

A. Pent-4-ynoic acid methyl ester (II-1B-2)

Pent-4-ynoic acid (10 g. 96.8 mmol) was dissolved in 500 mL of anhydrous methanol, and the solution was cooled to 0° C. before thionyl chloride (8.9 mL, 119 mmol) was added dropwise. The resulting reaction solution was warmed to room temperature and stirred under nitrogen overnight. Reaction was stopped and the solution was diluted with 1.5 L of dichloromethane and washed with 1 L of water. Organic solvents were removed using rotarvapor at 25° C. to afford the title compound (14.2, 100% crude yield). The crude product was taken into the next step without further purification.

B. 5-(3-Nitro-phenyl)-pent-4-ynoic acid methyl ester (Step I, II-2B)

1-Iodo-3-nitro-benzene (II-1B-1, 23.6 g, 94.8 mmol) and pent-4-ynoic acid methyl ester (II-1B-2, 14 g) were dissolved in 125 mL of anhydrous DMF. To this reaction solution were added triphenyl phosphine (1.99 g, 7.59 mmol), followed by palladium (II) acetate (0.85 g, 3.79 mmol), and copper (I) iodide (1.45 g, 7.61 mmol), and finally triethylamine (50 mL, 360 mmol) at 0° C. This resulting black reaction mixture was warmed to room temperature and stirred under nitrogen for 24 hours. The reaction was stopped by pouring it into 200 mL of ice water and 150 mL 3N HCl. The forming brown precipitate was filtered out and the mother liquid was extracted with 50 mL of ethyl acetate. Ethyl acetate solution was concentrated to dryness and the resulting solid was combined with the above-mentioned brown-slid, mixed with 450 mL of ethanol. The undissolved solid was removed via suction filtration, and the solution was concentrated, which was further purified by silica gel column chromatography using hexanes/ethyl acetate (15.8 g, 72.4% yield for two steps).

C. 5-(3-Aminophenyl)-pentanoic acid methyl ester (Step II, II-3B)

A reaction flask containing 5-(3-nitrophenyl)pent-4-ynoic acid methyl ester (II-2B, 15.2 g, 65.2 mmol) and Pd/C 10% wet (3.0 g) in 200 mL of methanol was shaken under hydrogen (45 psi) atmosphere at room temperature. After four hours, the reaction mixture was filtered through celite, and the methanol solution was concentrated to dryness. The resulting yellow residue was purified using silica gel column chromatography to afford the final compound (6.27 g, 46.4 yield).

D. 5-(3-Ethoxycarbonylamino-phenyl)-pentanoic acid methyl ester (Step III, II-4B)

5-(3-Amino-phenyl)-pentanoic acid methyl ester (II-3B, 5.1 g, 24.6 mmol) was dissolved in 50 mL of anhydrous dichloromethane and the solution was cooled by ice-water bath. To it was added ethyl diisopropyl amine (5.46 mL, 31,3 mmol), followed by ethyl chloroformate (2.77 mL, 29.0 mmol). This reaction solution was then warmed to room temperature and the stirred under nitrogen overnight. Reaction was stopped by removing dichloromethane. The residue was purified using silica gel column chromatography to yield the title compound (6.02 g, 87.6% yield).

E. 5-(3-Ethoxycarbonylamino-phenyl)-pentanoic acid (Step-IV, II-5B)

5-(3-Ethoxycarbonylamino-phenyl)-pentanoic acid methyl ester (II-4B, 5.47 g, 19.6 mmol) was dissolved in 65 mL of THF and 10 mL of water. To this clear yellow solution was added lithium hydroxide, and the resulting reaction mixture was heated to 55° C. Reaction completed after three hours. Heating was removed and the mixture was carefully neutralized, then further acidified with 3N HCl till pH=4-5. The mixture was separated into two layers. Aqueous phase was separated and extracted with 30 mL of dichloromethane. Organic phases were combined and solvents were totally removed to afford the title compound (4.98 g, 95.8% yield).

F. (5-Oxo-6,7,8,9-tetrahydro-5H-benzocyclohepten-2-yl)-carbamic acid ethyl ester (Step-V, II-6B)

Polyphosphoric acid (22 g) was taken in 75 mLof toluene and to it 25 g. of celite was added. While stirring. 5-(3-Ethoxycarbonylamino-phenyl)-pentanoic acid (II-5B,m 3.65 g, 13.76 mmol) was added and the reaction was kept under reflux. After two hours, the reaction mixture was cooled to room temperature, water was added while stirring vigorously. It was diluted with ethyl acetate and celite was filtered off and washed with ethyl acetate. Organic layer was separated and the aquoues layer was extracted with ethyl acetate (2×); drided over sodium sulfate. The solvents were evaporated and the residue was purified by flash silica gel chromatography to afford the title compound (3 g, 89% yield). MS-CI m/z: 248 (M+H).

G. 5-Hydroxymethyl-3-(5-oxo-6,7,8,9-tetrahydro-5H-benzocyclohepten-2-yl)-oxazolidin-2-one (Step-VI, II-7B)

The title compound was prepared according to the procedure described in Example 1, step IV using (5-Oxo-6,7,8,9-tetrahydro-5H-benzocyclohepten-2-yl)-carbamic acid ethyl ester. MS m/z: 276 (M+H).

H. Methanesulfonic acid 2-oxo-3-(5-oxo-6,7,8,9-tetrahydro-5H-benzocyclohepten-2-yl)-oxazolidin-5-ylmethyl ester (Example 3, Method B, Step VII). (Step-VII, II-8B)

The title compound was prepared according to the procedure described in Example 1, step V using 5-Hydroxymethyl-3-(5-oxo-6,7,8,9-tetrahydro-5H-benzocyclohepten-2-yl)-oxazolidin-2-one. MS m/z: 354 (M+H).

I. 5-Azidomethyl-3-(5-oxo-6,7,8,9-tetrahydro-5H-benzocyclohepten-2-yl)-oxazolidin-2-one (Example 3, Method B, Step VIII). (Step-VIII, II-9B)

The title compound was prepared according to the procedure described in Example 1, step VI using methanesulfonic acid 2-oxo-3-(5-oxo-6,7,8,9-tetrahydro-5H-benzocyclohepten-2-yl)-oxazolidin-5-ylmethyl ester. MS m/z: 301 (M+H).

J. 5-Aminomethyl-3-(5-oxo-6,7,8,9-tetrahydro-5H-benzocyclohepten-2-yl)-oxazolidin-2-one (Example 3, Method B, Step IX) (Step-1X, II-10B)

The title compound was prepared according to the procedure described in Example 1, step VII using 5-azidomethyl-3-(5-oxo-6,7,8,9-tetrahydro-5H-benzocyclohepten-2-yl)-oxazolidin-2-one. MS m/z: 275 (M+H).

K. N-[2-Oxo-3-(5-oxo-6,7,8,9-tetrahydro-5H-benzocyclohepten-2-yl)-oxazolidin-5-ylmethyl]-acetamide (Example 3, Method B) (Step-X, II-7)

The title compound was prepared according to the procedure described in Example 1, step VII using 5-aminomethyl-3-(5-oxo-6,7,8,9-tetrahydro-5H-benzocyclohepten-2-yl)-oxazolidin-2-one. MS m/z: 317 (M+H).

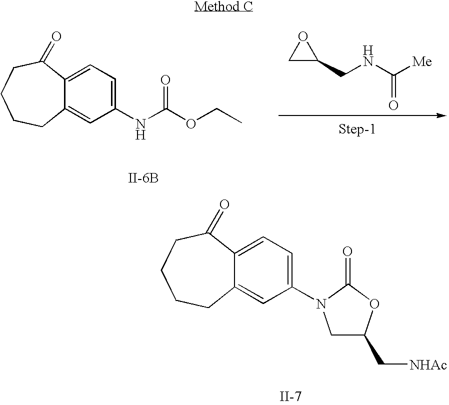

A. N-[2-Oxo-3-(5-oxo-6,7,8,9-tetrahydro-5H-benzocyclohepten-2-yl)-oxazolidin-5-ylmethyl]-acetamide

(5-Oxo-6,7,8,9-tetrahydro-5H-benzocyclohepten-2-yl)-carbamic acid ethyl ester (II-6B, 0.4 g, 1.62 mmol) was taken in 12 mL of tetrahydrofuran. To it n-butyllithium (1.6 M, 1.78 mmol) was added by dropwise at −78° C. After stirring the reaction mixture for 90 minutes at −78° C., (S)-N-oxiranylmethyl-acetamide (0.37 g, 3.24 mmol) taken in 2 Ml of tetrahydrofuran was added. The reaction mixture was slowly allowed to warm to room temperature. After stirring at room temperature for 30 minutes, it was heated to 60° C. for 2 hours. The reaction was cooled to room temperature, quenched with saturated ammonium chloride. Diluted with ethyl acetate, washed with saturated sodium bicarbonate solution, brine; dried over magnesium sulfate. The solvents were evaporated and the residue was purified by flash column silica gel chromatography to afford the title compound (0.25 g, 49% yield). MS-CI m/z: 317 (M+H).

### EXAMPLE 3

### N-[3-(8,9-Dihydro-7H-benzocyclohepten-2-yl)-2-oxo-oxazolidin-5-ylmethyl]-acetamide (III-2)

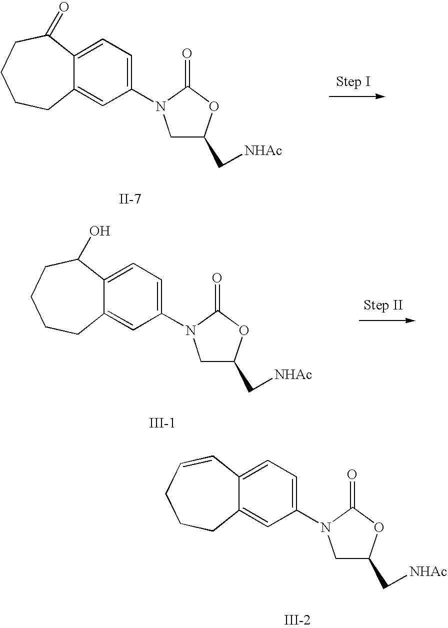

A. N-[3-(5-Hydroxy-6,7,8,9-tetrahydro-5H-benzocyclohepten-2-yl)-2-oxo-oxazolidin-5-ylmethyl]-acetamide (Step-I, III-1)

Sodium borohydride (107 mg, 2.84 mmol) was added at 0° C. to a solution of N-[2-Oxo-3-(5-oxo-6,7,8,9-tetrahydro-5H-benzocyclohepten-2-yl)-oxazolidin-5-ylmethyl]-acetamide (II-7, 450 mg, 1.42 mmol) in 10 mL of EtOH. The solution was stirred at 0° C. for 4 hrs. Saturated NaHCO₃ solution (10 mL) was added and the product was extracted with EtOAc (2×15 mL). The organic layer was dried over MgSO₄ and evaporated to give 420 mg of the title compound (93% Yield); MS m/e 300 (M—H₂O).

B. N-[3-(8,9-Dihydro-7H-benzocyclohepten-2-yl)-2-oxo-oxazolidin-5-ylmethyl]-acetamide (Step-II, III-2)

p-Toluenesulfonic acid monohydrate (960 mg, 5.03 mmol) was added to a solution of N-[3-(5-Hydroxy-6,7,8,9-tetrahydro-5H-benzocyclohepten-2-yl)-2-oxo-oxazolidin-5-ylmethyl]-acetamide (III-2) in 3 mL of DMF and 10 mL of toluene. The solution was refluxed overnight. The solvents were removed under reduced pressure, and the residue was re-dissolved in 15 mL of EtOAc. This was washed with 10 mL of saturated NaHCO₃ solution. The organic layer was washed with brine and dried over MgSO₄. Evaporation of the solvent gave 270 mg (72% Yield) title compound. MS m/e 300 (M).

### EXAMPLE 4

### N-[2-Oxo-3-(7-oxo-6,7,8,9-tetrahydro-5H-benzocyclohepten-2-yl)-oxazolidin-5-ylmethyl]-acetamide (IV-9)

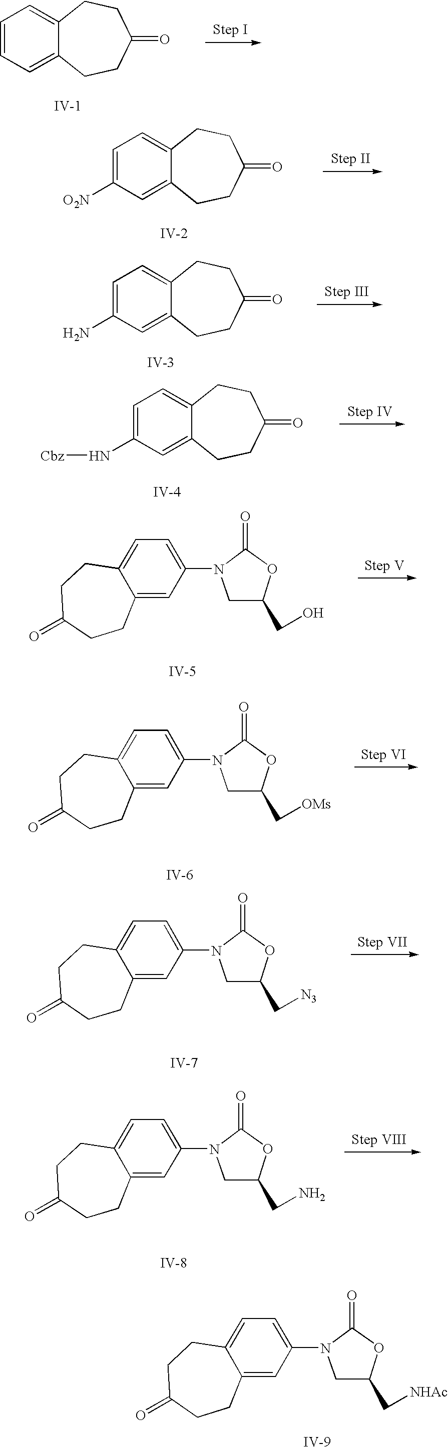

A. 5,6,8,9-Tetrahydro-benzocyclohepten-7-one (IV-1)

The title compound was prepared according to the procedure described in Bull. Chem. Soc. Japan 1979, 52, 273-274.

B. 2-Nitro-5,6,8,9-tetrahydro-benzocyclohepten-7-one (IV-2)

5,6,8,9-Tetrahydro-benzocyclohepten-7-one (8.0 g, 50 mmol) was dissolved in 200 mL of concentrated sulfuric acid and cooled in an ice-bath. A mixture of nitric acid (9 mL) and sulfuric acid (20 mL) was added dropwise and the resulting solution was stirred for 2 hours at 0° C. The resulting solution was poured into 500 mL of ice and extracted with dichloromethane (3×200 mL). The organic layer was washed with brine and dried over magnesium sulfate. Evaporation of the solvent gave 9.0 g of the product (89% yield). MS m/z: 206 (M+).

C. 2-Amino-5,6,8,9-tetrahydro-benzocyclohepten-7-one (IV-3)

2-Nitro-5,6,8,9-tetrahydro-benzocyclohepten-7-one (4.0 g, 19.5 mL) was dissolved in 40 mL of methanol. The solution was treated with 40 psi of hydrogen in the presence of 400 mg. of 10% Pd/C for 4 hours. The resulting solution was filtered through celite and the residue was washed with 50 mL of methanol and 50 mL of ethyl acetate. The solvent was removed to give 3.3 g (97% yield) of the product. MS m/z: 100 (M+).

D. (7-Oxo-6,7,8,9-tetrahydro-5H-benzocyclohepten-2-yl)-carbamic acid benzyl ester (IV-4)

2-Amino-5,6,8,9-tetrahydro-benzocyclohepten-7-one (2.44 g, 13.9 mmol) was dissolved in 40 mL of acetone and 20 mL of water. Sodium carbonate (2.34 g) was added and the solution was cooled in an ice-bath. Benzyl chloroformate (2.89 g, 16.7 mmol) was added dropwise. The resulting solution stirred at 0° C. for 2 hours and stirred at room temperature overnight. The mixture was poured into 150 mL of ice water and the resulting precipitation was collected by filtration. The solid was recrystallized from acetone and water to give 3.o g (70% yield) of the title product. MS m/z: 309.

E. 5-Hydroxymethyl-3-(7-oxo-6,7,8,9-tetrahydro-5H-benzocyclohepten-2-yl)-oxazolidin-2-one (IV-5)

(7-Oxo-6,7,8,9-tetrahydro-5H-benzocyclohepten-2-yl)-carbamic acid benzyl ester (4.0 g, 12.9 mmol) was dissolved in 50 mL of tetrahydrofuran and cooled to −78° C. A solution of lithium diisopropylamine (1.5 M in cyclohexane, 8.6 mL) was added and the resulting solution was stirred for 40 minutes. R-glycidylbutyrate (2.23 g in 15.5 mL of tetrahydrofuran) was added by dropwise. The resulting solution was stirred at −78° C. for 1 hour and then at room temperature overnight. Saturated ammonium chloride solution (25 mL), ethyl acetate (25 mL) and water (5 mL) were added. The organic layer was separated and the aqueous layer was extracted with ethyl acetate (3×25 mL). The organic layer was washed with brine and dried over magnesium sulfate. Evaporation of the solvent gave the title compound (5.2 g). MS m/z 276.

F. Methanesulfonic Acid 2-oxo-3-(7-oxo-6,7,8,9-tetrahydro-5H-benzocyclohepten-2-yl)-oxazolidin-5-ylmethyl ester (IV-6)

5-Hydroxymethyl-3-(7-oxo-6,7,8,9-tetrahydro-5H-benzocyclohepten-2-yl)-oxazolidin-2-one (4.0 g, 14.6 mmol) was dissolved in 80 mL of 1:1 mixture of dichloromethane and tetrahydrofuran and cooled in ince-bath. Triethylamine (2.93 g, 29 mmol) and mesityl chloride (2.5 g, 21.8 mmol) were added, and the resulting solution was stirred at 0° C. for 1 hour. Water (30 mL) was added and the aqueous layer was extracted with dichloromethane (3×50 m/L). The organic layer was washed with brine and dried over magnesium sulfate. The residue obtained after evaporation of the solvent was purified by flash silica gel chromatography to give 2.0 g (44% yield) of the title compound. MS m/z 354.

G. 5-Azidomethyl-3-(7-oxo-6,7,8,9-tetrahydro-5H-benzocyclohepten-2-yl)-oxazolidin-2-one (IV-7)

A mixture of methanesulfonic acid 2-oxo-3-(7-oxo-6,7,8,9-tetrahydro-5H-benzocyclohepten-2-yl)-oxazolidin-5-ylmethyl ester (2.0 g, 5.67 mmol) and sodium azide (1.84 g, 28.3 mmol) in 40 mL of dimethylformamide was heated at 70° C. for 16 hours. After cooling, water (80 mL) and ethyl acetate (40 mL) were added. The organic phase was washed with brine and dried over magnesium sulfate. Evaporation of the solvent gave 1.6 g (94% yield) of the title compound. MS m/z 300.

H. 5-Aminomethyl-3-(7-oxo-6,7,8,9-tetrahydro-5H-benzocyclohepten-2-yl)-oxazolidin-2-one (IV-8)

5-Azidomethyl-3-(7-oxo-6,7,8,9-tetrahydro-5H-benzocyclohepten-2-yl)-oxazolidin-2-one in 50 mL of ethyl acetate was treated with 40 psi of hydrogen overnight in the presence of 200 mg of 10% Pd/C. Fresh catalyst (200 mg) was added and hydrogenation was continued for another 24 hours. The reaction mixture was filtered through celite and the residue was washed with ethyl acetate and methanol (100 mL each). Evaporation of the solvent gave 1.6 g (87% yield) of the title compound. MS m/z 276.

I. N-[2-Oxo-3-(7-oxo-6,7,8,9-tetrahydro-5H-benzocyclohepten-2-yl)-oxazolidin-5-ylmethyl]-acetamide (IV-9)

5-Aminomethyl-3-(7-oxo-6,7,8,9-tetrahydro-5H-benzocyclohepten-2-yl)-oxazolidin-2-one (1.6 g, 5.8 mmol) was dissolved in pyridine (8 m]L) and cooled in an ice-bath. Acetic anhydride (2.0 mL, 21.5 mmol) was added and the mixture was stirred at 0° C. for 1 hour and at room temperature overnight. Excess reagents were removed under reduced pressure, and the residue was chromatographed using silica gel to give 800 mg (44% yield) of the title compound. MS m/z 317.

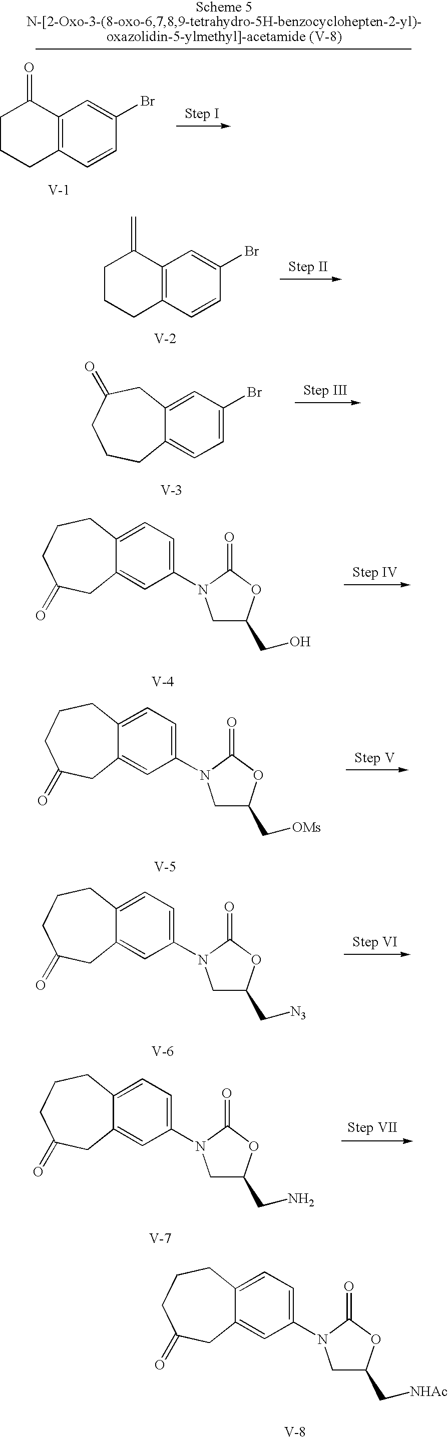

A. 7-Bromo-1-methylene-1,2,3,4-tetrahydro-naphthalene (Step-I, V-2)

7-Bromo-3,4-dihydro-2H-naphthalen-1-one (1.62 g, 7.2 mmol) was dissolved in tetrahydrofuran (18 mL) and cooled to 0° C. To it Tebbe reagent (0.5 M in toluene, 18 mL, 9 mmol) was added slowly and the reaction was stirred for 3 hours at 0° C. The reaction was quenched with 0.1 N sodium hydroxide by drop wise addition. Diluted with ethyl acetate, organic layer was separated and dried over sodium sulfate. The crude material was purified by flash silica gel chromatography to afford the title compound (Yield: 54%). LC-MS m/z: 225 (M+H).

B. 3-Bromo-5,7,8,9-tetrahydro-benzocyclohepten-6-one (Step-II, V-3)

Silver nitrate was dissolved in methanol (36 mL) and refluxed for 1 hour until all the material had dissolved. 7-bromo-1-methylene-1,2,3,4-tetrahydro-naphthalene (0.87 g, 3.91 mmol) in methanol (24 mL) and iodine (0.992 g, 3.91 mmol) taken in a separate flash was added to the silver nitrate solution. The reaction was kept under reflux for 2 hours. The reaction mixture was cooled to room temperature, filtered through celite. To it 1 N hydrochloric acid was added and methanol was removed under vacuum. The aqueous residue was dissolved in ether and washed with 10% sodium thiosulfate, brine and dried over sodium sulfate and concentrated. The residue was purified by flash silica gel chromatography to obtain the title compound (0.48 g, 52% yield). LC-MS m/z: 239 (M+H).

C. 5-Hydroxymethyl-3-(8-oxo-6,7,8,9-tetrahydro-5H-benzocyclohepten-2-yl)-oxazolidin-2-one (Step-III, V-4)

3-Bromo-5,7,8,9-tetrahydro-benzocyclohepten-6-one (0.070 g, 0.29 mmol), 5-hydroxymethyl-oxazolidin-2-one (0.034 g, 0.29 mmol), copper (I) iodide (0.011 g, 0.059 mmol), trans-1,2-diaminocyclohexane (7 μL, 0.059 mmol), potassium carbonate (0.084 g, 0.61 mmol) and dimethylformamide (0.5 mL) were combined. The mixture was purged with stirring another four times and heated to 105° C. overnight under nitrogen. The reaction mixture was diluted with ethyl acetate and water, the organic layer was washed with 1 M hydrochloric acid (2×), brine, dried over sodium sulfate and concentrated in vacuo to give Yield: 51%; LC-MS m/z: 276 (M+H).

D. Methanesulfonic acid 2-oxo-3-(8-oxo-6,7,8,9-tetrahydro-5H-benzocyclohepten-2-yl)-oxazolidin-5-ylmethyl ester (Step-IV, V-5)

5-Hydroxymethyl-3-(8-oxo-6,7,8,9-tetrahydro-5H-benzocyclohepten-2-yl)-oxazolidin-2-one (1.72 mmol), mesityl chloride (200 μL, 2.58 mmol), triethyl amine (0.44 mL, 3.44 mmol) and dichloromethane (8.6 mL) were combined and stirred at room temperature overnight. The solution was diluted with ethyl acetate, washed with 1 M hydrochloric acid (2×), brine, dried over sodium sulfate and concentrated. The residue was triturated with diethyl ether (2×) and dried in vacuo to give. Yield: 72% over two steps. LC-MS m/z: 354 (M+H).

E. 5-Azidomethyl-3-(8-oxo-6,7,8,9-tetrahydro-5H-benzocyclohepten-2-yl)-oxazolidin-2-one (Step-V, V-6)

Methanesulfonic acid 2-oxo-3-(8-oxo-6,7,8,9-tetrahydro-5H-benzocyclohepten-2-yl)-oxazolidin-5-ylmethyl ester (0.44 g, 1.25 mmol), sodium azide (0.31 g, 4.7 mmol) and dimethyl formamide (6.25 mL) were combined and and heated at 80° C. for 2 hours. The solution was cooled to room temperature and diluted with ethyl acetate. The organic phase was washed with water (2×), brine and transferred to a hydrogenation bottle. 10% Palladium on carbon (50 mg) was added and the mixture was hydrogenated at 50 psi for 2.5 hours. The solution was diluted with methanol, filtered through a pad of celite and concentrated in vacuo to give Yield: 40%; LC-MS: 301 (M+H).

F. 5-Aminomethyl-3-(8-oxo-6,7,8,9-tetrahydro-5H-benzocyclohepten-2-yl)-oxazolidin-2-one (Step-VI, V-7)

5-Azidomethyl-3-(8-oxo-6,7,8,9-tetrahydro-5H-benzocyclohepten-2-yl)-oxazolidin-2-one (0.12 g, 0.4 mmol), Pd/C (50 mg), and methanol (5 mL) were combined in a hydrogenation bottle. The mixture was hydrogenated at 50 psi for 2.5 hours. The solution was diluted with methanol, filtered through a pad of celite and concentrated in vacuo to give. Yield: 71%; LC-MS m/z: 275 (M+H).

G. N-[2-Oxo-3-(8-oxo-6,7,8,9-tetrahydro-5H-benzocyclohepten-2-yl)-oxazolidin-5-ylmethyl]-acetamide (Step-VII, V-8)

5-Aminomethyl-3-(8-oxo-6,7,8,9-tetrahydro-5H-benzocyclohepten-2-yl)-oxazolidin-2-one (0.088 g, 0.28 mmol), acetic anhydride (0.37 μL, 0.39 mmol), pyridine (66 μL, 0.84 mmol) and dichloromethane (2 mL) were combined at room temperature and stirred for 20 minutes. The solution was diluted with ethyl acetate, washed with 1 M hydrochloric acid (2×), brine, dried over sodium sulfate and concentrated. The residue was dissolved in methanol and filtered through a DOWEX 1×4-100 ion exchange resin plug (strongly basic anion, 4% cross-linking) and concentrated in vacuo to give the acetylated compound. A solution of the acetylated compound in 4 N hydrochloric acid in dioxane was then stirred at room temperature under nitrogen for 20 minutes. The solvent was decanted and the residue was dried under high vacuum to give the title compound. Yield: 71%; LC-MS m/a: 317 (M+H).

### EXAMPLE 6

### N-[3-(6,7-dihydro-5H-benzocyclohepten-2-yl)-2-oxo-oxazolidin-5-ylmethyl]acetamide (XIV-2)

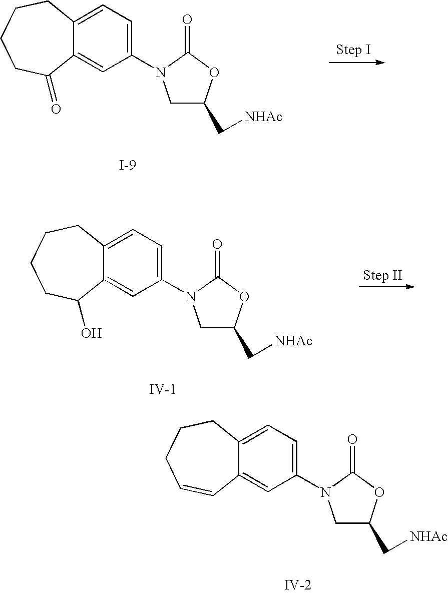

A. N-[3-(9-Hydroxy-6,7,8,9-tetrahydro-5H-benzocyclohepten-2-yl)-2-oxo-oxalidin-5-ylmethyl]acetamide (Step I, VI-4)

To N-[2-Oxo-3-(9-oxo-6,7,8,9-tetrahydro-5H-benzocyclohepten-2-yl)oxalidin-5-ylmethyl]acetamide (I-9, 0.50 grams, 1.58 mmol) in ethanol (12 mL) cooled to 0° C. was added sodium borohydride (0.12 g, 3.16 mmol, 2.0 eq.) portionwise and the resulting mixture stirred to 0° C. After 2 hours the reaction was quenched with sat. sodium bicarbonate and aqueous extracted with ethyl acetate (2×), organics combined and washed with brine, dried over magnesium sulfate, filtered and concentrated. The isolated residue was subjected to chromatography using Combiflash system, eluting with MeOH/CH₂Cl₂ gradient (0-6% MeOH over 1 hour) to afford the title compound. (Yield: 0.32 g, 64%). MS-APCI (m/z+): 301.

B. N-[3-(6,7-dihydro-5H-benzocyclohepten-2-yl)-2-oxo-oxalidin-5-ylmethyl]acetamide (Step II, VI-2)

N-[3-(9-Hydroxy-6,7,8,9-tetrahydro-5H-benzocyclohepten-2-yl)-2-oxo-oxalidin-5-ylmethyl]acetamide (0.32 g, 1.01 mmol) was dissolved in DMF (2.5 mL) and toluene (7.5 mL) and treated with p-TSA (0.75 g, 9.92 mmol, 3.9 eq.) and heated at reflux overnight. Heat was then removed and majority of solvent removed on rotary evaporator. Ethyl acetate was added, organics were washed with saturated sodium bicarbonate, followed by brine, dried over magnesium sulfate, filtered and concentrated. The isolated residue was subjected to chromatography using Combiflash system, eluting with MeOH/EtOAc gradient (0-4% MeOH over 1 hour) to afford the title compound. (Yield: 0.170 g, 56%). MS-APCI (m/z+): 301MS-APCI (m/z+): 257,301 (M+H).

### EXAMPLE 7

### N-[2-Oxo-3-(6-oxo-6,7,8,9-tetrahydro-5H-benzocyclohepten-2-yl)-oxazolidin-5-ylmethyl]-acetamide (VII-6)

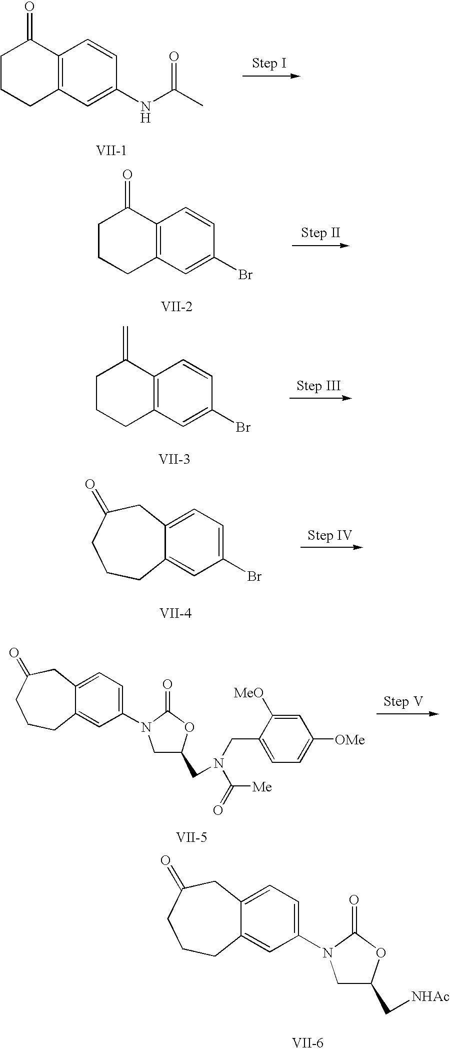

A. 6-Bromo-3,4-dihydro-2H-naphthalen-1-one (Step I, VII-2)

The title compound was prepared according to the procedure described in the literture. (References: J. Medicinal Chemistry, 1994, 37, 3485 and J. Organic Chemistry 1962, 27, 70-76).

B. 6-Bromo-1-methylene-1,2,3,4-tetrahydro-naphthalene (Step II, VII-3)

To a suspension of methyltriphenylphosphonium iodide (2.92 g, 7.23 mmol) in tetrahydrofuran (10 mL) was added potassium hexamethyldisilazide and the mixture was stirred for 20 minutes at 0° C. To it 6-Bromo-3,4-dihydro-2H-naphthalen-1-one (XV-2, 1.48 g, 6.57 mmol) was added all at once, and the misture was allowed to warm slowly to room temperature over 4 hours. Saturated ammonium chloride was added, and the mixture was extracted with ethyl acetate (2×100 mL). The combined extracts were washed with brine, dried over magnesium sulfate, and concentrated. The residue was chromatographed over silica gel, eluting with 3:1 hexane:ethyl acetate, to give the title compound (1.18 g, 81% yield). H¹NMR (400 MHz, CDCl₃): δ 1.84-1.91 (m, 2H), 2.52-2.58 (m, 2H), 2.83 (m, 2H), 4.99 (s, 1H), 5.47 (s, 1H), 7.25-7.29 (m, 2H), 7.50-7.52 (m, 1H); MS m/z: 225 (M+H).

C. 2-Bromo-5,7,8,9-tetrahydro-benzocyclohepten-6-one (Step III, VII-4)

Silver nitrate (2.4 g, 14.1 mmol) was dissolved in methanol (50 mL) and refluxed for 1 hour until the solids had dissolved. A solution of 6-Bromo-1-methylene-1,2,3,4-tetrahydro-naphthalene (1.58 g, 7.08 mmol) in methanol (30 mL) was treated with iodine and then added all at once to the hot silver nitrate solution. The suspension was refluxed for 4.5 hours and cooled to room temperature. The mixture was filtered through a fiberglass pad; the filtratrate was treated with 3 N hydrochloric acid (10 mL) and concentrated. The residue was partitioned between water and ethyl acetate and the organic layer was separated, washed with brine, 10% sodium bisulfite and dried over magnesium sulfate and concentrated. The crude product was chromatographed over silica gel, eluting with 3:1 hexane:ethyl acetate, to give the title compound (1.3 g, 77% yield). H¹NMR (400 MHz, CDCl₃): δ 1.97-2.04 (m, 2H), 2.53-2.57 (m, 2H), 2.89-2.92 (m, 2H), 3.66 (s, 2H), 7.01 (d, 1H), 7.29-7.32 (m, 2H); MS m/z 241 (M+H).

D. N-(2,4-Dimethoxy-benzyl)-N-[2-oxo-3-(6-oxo-6,7,8,9-tetrahydro-5H-benzocyclohepten-2-yl)-oxazolidin-5-ylmethyl]-acetamide (Step IV, VII-5)

A solution of 2-Bromo-5,7,8,9-tetrahydro-benzocyclohepten-6-one (0.63 g, 2.63 mmol), N-(2,4-Dimethoxy-benzyl)-N-(2-oxo-oxazolidin-5-ylmethyl)-acetamide (0.81 g, 2.63 mmol), potassium carbonate (0.76 g, 5.5 mmol), and dimethylformamide (5 mL) was purged with nitrogen and evacuated several times. The 1,2-diaminocyclohexane (0.06 mL, 0.5 mmol) and cuprous iodide (0.1 g, 0.52 mmol) were added, and the purge/evacuation process was repeated several more times. The mixture was then heated to 110° C. under nitrogen for 20 hours. The suspension was cooled to room temperature and diluted with ethyl acetate:methanol: 1 N hydrochloric acid. The organic layer was separated, washed with 1 N hydrochloric acid and brine, and dried over magnesium sulfate. Concentration gave a residue, which was chromatographed over silica gel, eluting with ethyl acetate:dichloromethane:methanol (15:2:1), to give the title compound (0.18 g). MS m/z 467 (M+H).

E. N-[2-Oxo-3-(6-oxo-6,7,8,9-tetrahydro-5H-benzocyclohepten-2-yl)-oxazolidin-5-ylmethyl]-acetaminde (Step V, VII-6)

A mixture of N-(2,4-Dimethoxy-benzyl)-N-[2-oxo-3-(6-oxo-6,7,8,9-tetrahydro-5H-benzocyclohepten-2-yl)-oxazolidin-5-ylmethyl]-acetamide (0.2 g, 0.43 mmol) in trifluoroacetic acid (4 mL) was stirred at room temperature for 2.5 hours. The solvent was evaporated, and the residue was chromatographed over silica gel to yield title compound. MS m/z 317 (M+H).

### EXAMPLE 8

### N-[3-(6-Bromo-5-oxo-6,7,8,9-tetrahydro-5H-benzocyclohepten-2-yl)-2-oxo-oxazolidin-5-ylmethyl]-acetamide

To a solution of N-[2-Oxo-3-(5-oxo-6,7,8,9-tetrahydro-5H-benzocyclohepten-2-yl)-oxazolidin-5-ylmethyl]-acetamide (Example 3, step G-7, 1.0 g, 31.65 mmol) in 30 mL of chloroform was added 0.162 mL (31.65 mmol) of bromine. This was stirred at room temperature overnight. Saturated aqueous sodium bicarbonate (15 mL) was added followed by 20 mL of methylene chloride. The layers were separated and the aqueous layer was extracted with methylene chloride (3×20 mL). The combined organic layers were dried (MgSO4) and concentrated under reduced pressure to provide 1.3 g product as an off white foam.

### EXAMPLE 9

### N-[2-Oxo-3-(5-oxo-2,3,4,5-tetrahydro-benzo[b]thiepin-8-yl)-oxazolidin-5-ylmethyl]-acetamide (IX-6)

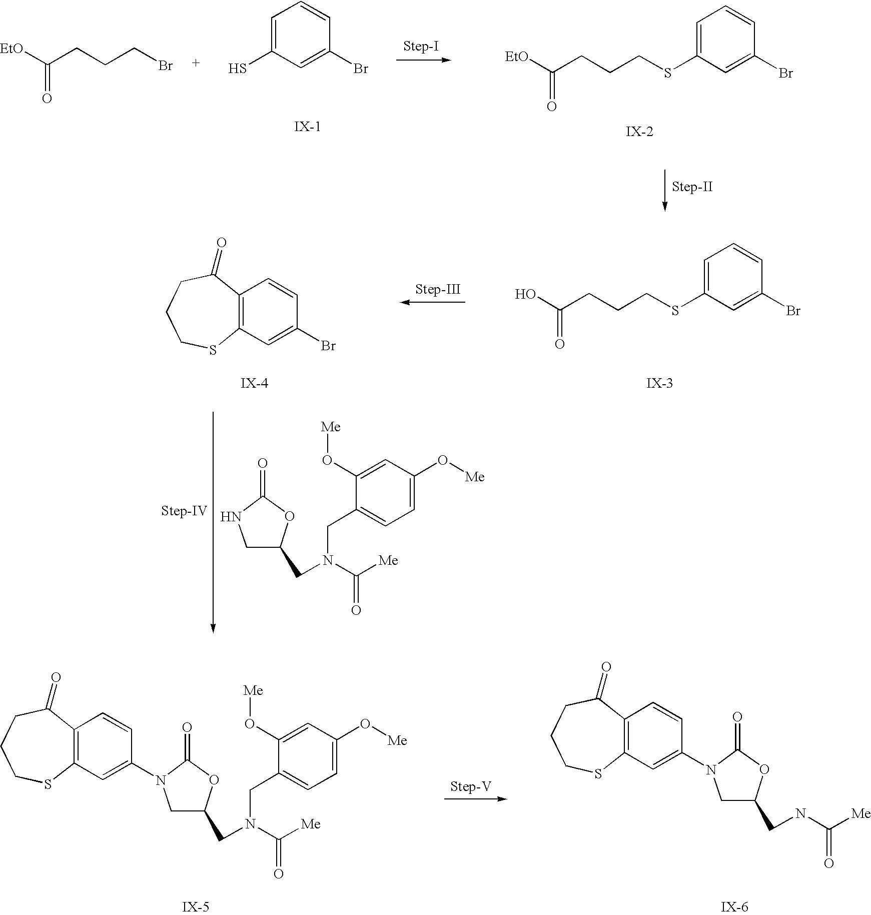

A. 4-(3-Bromo-phenylsulfanyl)-butyric acid ethyl ester. (Step I, IX-2)

A solution of 3-bromothiol (IV-1, 500 mg, 2.64 mmol), ethyl 4-bromobutyrate (378 μl, 2.64 mmol), and cesium carbonate (2.58 g, 7.92 mmol) in dimethylformamide (5 ml) was stirred at room temperature under nitrogen for 1 hour. The reaction was then diluted with diethyl ether, washed with 1 M hydrochloric acid (2×50 mL), brine (50 mL), dried over sodium sulfate and concentrated. GC-MS: 302/304 (M+). The crude product was used without any further purification.

B. 4-(3-Bromo-phenylsulfanyl)-butyric acid. (Step II, IX-3)

A solution of 4-(3-bromo-phenylsulfanyl)-butyric acid ethyl ester (IV-2, crude from previous step), 2 M aqueous lithium hydroxide (5 mL) in tetrahydrofuran (5 mL) was stirred at 50° C. for 1.5 hours. The reaction was quenched with 1 M hydrochloric acid and diluted with ether. The organic layer was washed with 1 M hydrochloric acid, brine, dried over sodium sulfate and concentrated in vacuo to give the title compound (581 mg, 98% over two steps). H¹NMR (400 MHz, CDCl₃): δ 1.96 (quintet, J=7 Hz, 2H), 2.52 (t, J=8 Hz, 2H), 2.97 (t, J=8 Hz, 2H), 7.13 (t, J=8 Hz, 1H), 7.23 (dt, J=1.7, 8 Hz, 1H), 7.29 (dt, J=1.7, 8 Hz, 1H) 7.45 (d, J=2 Hz, 1H). LC-MS: m/z 273.5 (M−1), 321.4 (M-1+HCOOH).

C. 8-Bromo-3,4-dihydro-2H-benzo[b]thiepin-5-one. (Step-III, IX-4)

A mixture of 4-(3-bromo-phenylsulfanyl)-butyric acid (IV-3, 555 mg) and polyphosphoric acid (approximately 2 mL) was stirred at 100° C. under nitrogen overnight. The mixture was diluted with ether/water, washed with water (2×), dried over sodium sulfate and concentrated to give a cloudy oil that was purified on a 40S Biotage column (0 to 100% ethyl acetate/hexanes over 25 minutes) to give 348 mg (67% Yield) of the title compound. H¹NMR (400 MHz, CDCl₃): δ 2.26 (quintet, J=7 Hz, 2H), 2.97 (t, J=7 Hz, 2H), 3.02 (t, J=6.6 Hz, 2H), 7.36 (dd, J=8.7, 2 Hz, 1H), 7.63 (d, J=1.7 Hz, 1H), 7.68 (d, J=8.7 Hz, 1H).

D. N-(2,4-Dimethoxy-benzyl)-N-[2-oxo-3-(5-oxo-2,3,4,5-tetrahydro-benzo[b]thiepin-8-yl)-oxazolidin-5-ylmethyl]-acetamide (Step 1V, IX-5)

N-(2,4-Dimethoxy-benzyl)-N-(2-oxo-oxazolidin-5-ylmethyl)-acetamide (410 mg, 1.33 mmol), 8-bromo-3,4-dihydro-2H-benzo[b]thiepin-5-one(IV-4, 342 mg, 1.33 mmol), racemic trans-1,2 diaminocyclohexane (32 □1, 0.27 mmol), and potassium carbonate (386 mg, 2.79 mmol) were dissolved in dioxane (1.5 mL). The mixture was purged with nitrogen six times. Copper (I) iodide (51 mg, 0.27 mmol) was added; the mixture was purged another six times and stirred at 110° C. for 20 hours. It was cooled to room temperature and diluted with ethyl acetate/methanol/1 M hydrochloric acid. The organic layer was washed with 1 M hydrochloric acid (3×), dried over sodium sulfate and concentrated in vacuo to give the title compound (586 mg, 91% Yield). H¹NMR (400 MHz, CDCl₃): δ 2.21 (s, 3H), 2.25 (quintet, 2H), 2.98 (t, J=6.7 Hz, 2H), 3.03 (t, J=7.5 Hz, 2H), 3.40 (dd, J=6.5, 14.5 Hz, 1H), 3.65-3.9 (m, 2H), 3.78 (s, 3H), 3.79 (s, 3H), 4.03 (t, J=9 Hz, 1H), 4.5-4.65 (q, J=16.6 Hz, 2H, benzylic), 4.85 (m, 1H), 6.44 (m, 2H), 7.95 (d, J=8.7 Hz, 1H), 7.46 (dd, J=2.5, 8.7 Hz, 1H), 7.59 (d, J=2.5 Hz, 1H), 7.87 (d, J=8.7 Hz, 1H). LC-MS: m/z 486.1 (M+1).

E. N-[2-Oxo-3-(5-oxo-2,3,4,5-tetrahydro-benzo[b]thiepin-8-yl)-oxazolidin-5-ylmethyl]-acetamide (Step V, IX-6)

N-(2,4-Dimethoxy-benzyl)-N-[2-oxo-3-(5-oxo-2,3,4,5-tetrahydro-benzo[b]thiepin-8-yl)-oxazolidin-5-ylmethyl]-acetamide (IV-5, 125 mg, 0.26 mmol) was stirred in trifluoroacetic acid (3 mL) for 3.5 hours. The solution was concentrated in vacuo and the residue was triturated with pentane (3×). The residue was recrystallized from methanol/ethyl acetate/pentane to give (23 mg, 27% Yield) the title compound. H¹NMR (400 MHz, CDCl₃): δ 2.06 (s, 3H), 2.23 (quintet, J=6.6 Hz, 2H), 2.94 (t, J=7 Hz, 2H), 2.99 (t, J=7 Hz, 2H), 3.67 (m, 2H), 3.80 (m, 2H), 4.81 (m, 1H), 7.12 (broad triplet, J=5.8 Hz, 1H, NH), 7.35 (dd, J=2.0, 8.7 Hz, 1H), 7.57 (d, J=2.5 Hz, 1H), 7.80 (d, J=8.7 Hz, 1H). LC-MS: m/z 335.4 (M+1), 379.5 (M−1+HCOOH).

### EXAMPLE 10

### N-[2-Oxo-3-(1,1,5-trioxo-2,3,4,5-tetrahydro-1H-1λ⁶-benzo[b]thiepin-8-yl)-oxazolidin-5-ylmethyl]-acetamide (V-2)

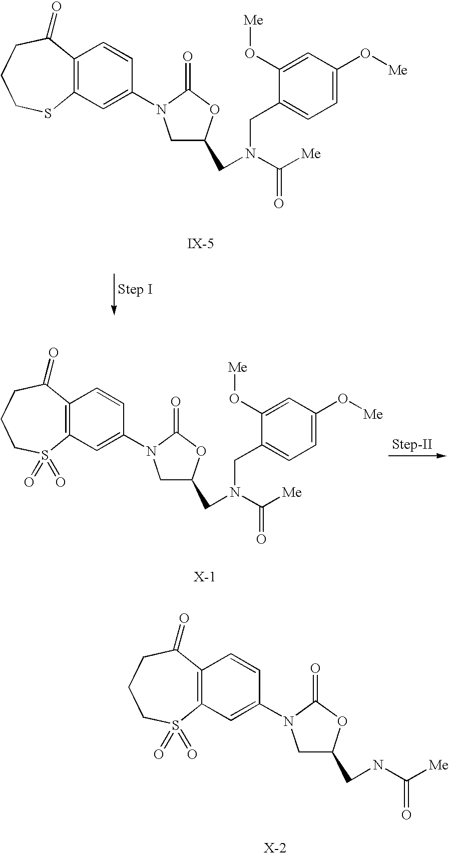

A. N-(2,4-Dimethoxy-benzyl)-N-[2-oxo-3-(1,1,5-trioxo-2,3,4,5-tetrahydro-1H-1λ⁶-benzo[b]thiepin-8-yl)-oxazolidin-5-ylmethyl]-acetamide (Step I, X-1)

To a dichloromethane solution (5 mL) of N-(2,4-dimethoxy-benzyl)-N-[2-oxo-3-(5-oxo-2,3,4,5-tetrahydro-benzo[b]thiepin-8-yl)-oxazolidin-5-ylmethyl]-acetamide (IX-5, 122 mg, 0.25 mmol) was added meta-chloroperoxybenzoic acid (140 mg, 0.63 mmol, 2.50 equivalents, 77% pure) and stirred under nitrogen at room temperature overnight. The solution was diluted with ethyl acetate, washed with saturated sodium bicarbonate (2×), brine, dried over sodium sulfate and concentrated in vacuo to give the title compound as a semisolid (99 mg, 78% Yield). H¹NMR (400 MHz, CDCl₃): δ 2.16 (quintet, 2H), 2.21 (s, 3H), 3.05 (m, 2H), 3.43-3.48 (m, 3H), 3.76 (s, 3H), 3.80 (s, 3H), 3.77-3.9 (m, 2H), 4.48-4.65 (app q, 2H), 4.91 (m, 1H), 6.41-6.44 (m, 2H), 6.96 (d, J=7.9 Hz, 1H), 7.73 (d, J=8.7 Hz, 1H), 7.92 (d, J=2.5 Hz, 1H), 8.07 (dd, J=2.5, 8.7 Hz, 1H). LC-MS: m/z 517.6 (M+1), 561.6 (M−1+HCOOH).

B. N-[2-Oxo-3-(1,1,5-trioxo-2,3,4,5-tetrahydro-1H-1λ⁶-benzo[b]thiepin-8-yl)-oxazolidin-5-ylmethyl]-acetamide (Step-II, X-2)

The title compound was prepared from N-(2,4-Dimethoxy-benzyl)-N-[2-oxo-3-(1,1,5-trioxo-2,3,4,5-tetrahydro-1H-16-benzo[b]thiepin-8-yl)-oxazolidin-5-ylmethyl]-acetamide (V-1) (66 mg, 94% Yield). H¹NMR (500 MHz, CDCl₃): δ 2.06 (s, 3H), 2.21 (m, 2H), 3.10 (m, 2H), 3.51 (t, J=6 Hz, 2H), 3.6-3.9 (m, 3H), 4.2 (t, J=9 Hz, 1H), 6.10 (broad triplet, 1H, NH), 7.79 (d, J=9 Hz, 1H), 8.02 (d, J=2 Hz, 1H), 8.11 (dd, J=2 Hz, J=9 Hz, 1H). LC-MS: m/z 367.6 (M+1).

### EXAMPLE 11

### N-[2-Oxo-3-(5-oxo-2,3,4,5-tetrahydro-benzo[b]oxepin-7-yl)-oxazolidin-5-ylmethyl]-acetamide (XI-6)

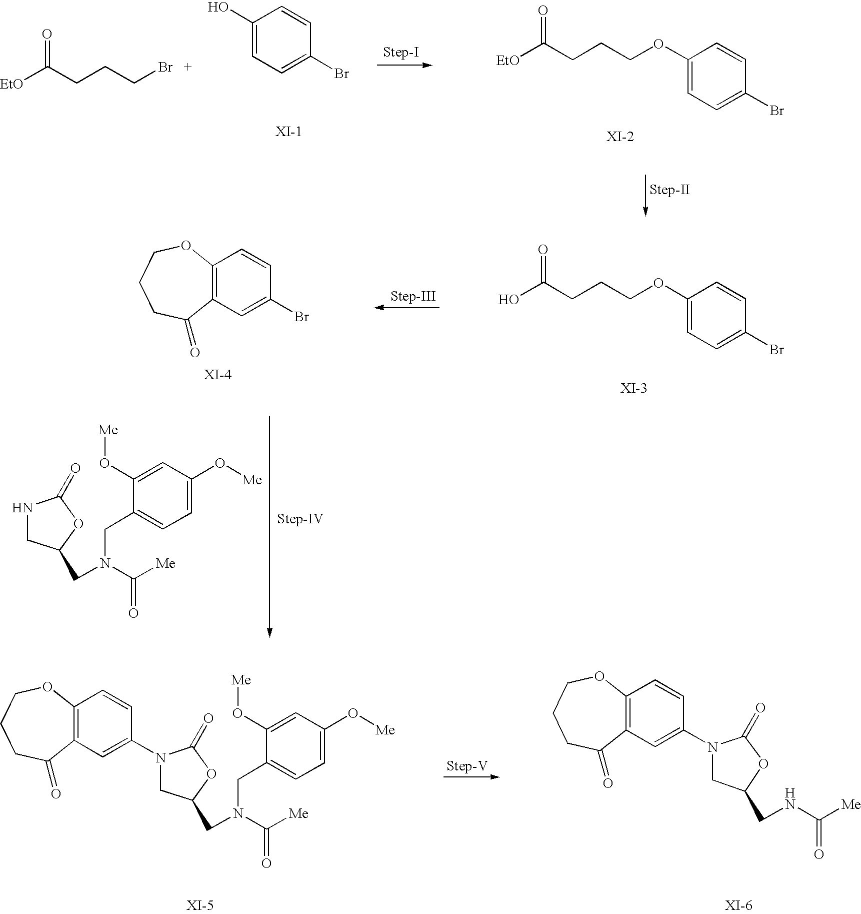

A. 4-(4-Bromo-phenoxy)-butyric acid ethyl ester (XI-2, Step I)

The title compound was prepared from p-bromophenol (XI-1) and ethyl 4-bromobutyrate according to the procedure as described in Example 9, step I.

Yield: 2.19 g (82%). H¹NMR (400 MHz, CDCl₃): δ 1.24 (t, 3H), 2.09 (quintet, 2H), 2.50 (t, 2H), 3.97 (t, 2H), 4.15 (q, 2H), 6.75 (d, 2H), 7.35 (d, 2H).

B. 4-(4-Bromo-phenoxy)-butyric acid (Step II, XI-3)

The title compound was prepared from 4-(4-bromo-phenoxy)-butyric acid ethyl ester (XI-2) according to the procedure as described in Example 9, step II. Yield: 1.86 g (94%). H¹NMR (400 MHz, CDCl₃): δ 2.1 (quintet, 2H), 2.58 (t, 2H), 3.97 (t, 2H), 6.75 (d, 2H), 7.35 (d, 2H).

C. 7-Bromo-3,4-dihydro-2H-benzo[b]oxepin-5-one (Step III, XI-4)

The title compound was prepared from 4-(4-bromo-phenoxy)-butyric acid ester (XI-3) according to the procedure as described in Example 9, step III. Yield: 0.17 g (36%). H₁NMR (400 MHz, CDCl₃): δ 2.2 (quintet, 2H), 2.88 (t, 2H), 4.22 (t, 2H), 6.95 (d, 1H), 7.49 (dd, 1H), 7.83 (d, 1H). GC-MS (EI): 240/242 (M+)

D. N-(2,4-Dimethoxy-benzyl)-N-[2-oxo-3-(5-oxo-2,3,4,5-tetrahydro-benzo[b]oxepin-7-yl)-oxazolidin-5-ylmethyl]-acetamide (Step 1V, XI-5)

The title compound was prepared from N-(2,4-dimethoxy-benzyl)-N-(2-oxo-oxazolidin-5-ylmethyl)-acetamide and 7-bromo-3,4-dihydro-2H-benzo[b]oxepin-5-one (XI-4) according to the procedure as described in Example p, step 1V (149 mg, 58% Yield). H¹NMR (400 MHz, CD₃OD): δ 2.15 (m, 2H), 2.21 (s, 3H), 2.82 (t, 2H), 3.50-3.55 (m, 1H), 3.62-3.8 (m, 3H), 3.74 (s, 3H), 3.80 (s, 3H), 3.98-4.1 (m, 2H), 4.18 (t, 2H), 4.49-4.69 (m, 2H), 4.83 (m, 1H), 6.4-6.54 (m, 2H), 7.0-7.1 (m, 2H), 7.65-7.75 (m, 2H). LC-MS: m/z 469.2 (M+1), 513.2 (M−1+HCOOH).

E. N-[2-Oxo-3-(5-oxo-2,3,4,5-tetrahydro-benzo[b]oxepin-7-yl)-oxazolidin-5-ylmethyl]-acetamide (Step V, XI-6)

The title compound was prepared from N-(2,4-dimethoxy-benzyl)-N-[2-oxo-3-(5-oxo-2,3,4,5-tetrahydro-benzo[b] oxepin-7-yl)-oxazolidin-5-ylmethyl]-acetamide (XI-5) according to the procedure as described in example 9, step V (39 mg, 39%). H₁NMR (400 MHz, CD₃OD): δ 1.95 (s, 3H), 2.15 (m, 2H), 2.82 (t, 2H), 3.55 (br s, 2H), 3.80 (t, 1H), 4.13 (t, 1H), 4.19 (t, 2H), 4.77 (m, 1H), 7.08 (d, 1H), 7.73 (m, 2H), 8.41 (br s, 1H, NH). LC-MS: m/z 319.3 (M+1).

### EXAMPLE 12

### 5 N-[2-Oxo-3-(5-oxo-2,3,4,5-tetrahydro-benzo[b]oxepin-8-yl)-oxazolidin-5-ylmethyl]-acetamide

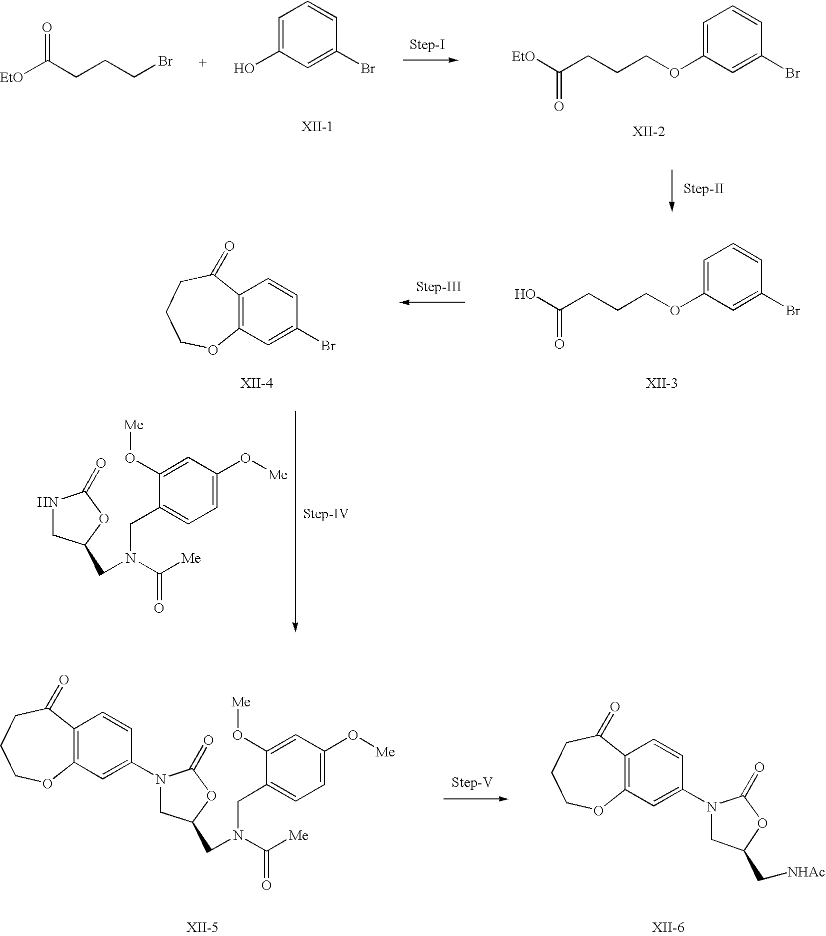

A. 4-(3-Bromo-phenoxy)-butyric Acid Ethyl Ester (Step I, XII-2)

The title compound was prepared from meta-bromophenol (VII-1) and ethyl 4-bromobutyrate according to the same procedure as described in Example 9, step I (Yield: 2.20 g, 78%). H¹NMR (500 MHz, CDCl₃): δ 1.27 (t, J=6.7 Hz, 3H), 2.12 (quintet, J=7.2 Hz, 2H), 2.52 (t, J=7.2 Hz, 2H), 4.00 (t, J=6.2 Hz, 2H), 4.15 (q, J=7.2 Hz, 2H), 6.83 (dd, J=2.1, 7.8 Hz, 1H), 7.05-7.16 (m, 3H). LC-MS: m/z 287.2/289.2 (M+1).

B. 4-(3-Bromo-phenoxy)-butyric acid (Step II, XII-3)

The title compound was prepared from 4-(3-bromo-phenoxy)-butyric acid ethyl ester (VII-2) according to the procedure as described in Example 9, step II (Yield: 1.68 g, 84%). H¹NMR (400 MHz, CDCl₃): δ 2.10 (quintet, J=7.2 Hz, 2H), 2.57 (t, J=7.2 Hz, 2H), 3.99 (t, J=6.2 Hz, 2H), 6.81 (dd, J=2.1, 7.8 Hz, 1H), 7.02-7.14 (m, 3H).

C. 8-Bromo-3,4-dihydro-2H-benzo[b]oxepin-5-one (Step III, XII-4)

The title compound was prepared from 4-(3-bromo-phenoxy)-butyric acid (See Example 7, step III) according to the procedure as described in Example 9, step III (Yield: 1.30 g, 83%). H¹ NMR (400 MHz, CDCl₃): δ 2.20 (quintet, J=7 Hz, 2H), 2.87 (t, J=7 Hz, 2H), 4.23 (t, J=6.6 Hz, 2H), 7.21-7.26(m, 2H), 7.62 (d,J=8.7 Hz, 1H).

D. N-(2,4-Dimethoxy-benzyl)-N-[2-oxo-3-(5-oxo-2,3,4,5-tetrahydro-benzo[b]oxepin-8-yl)-oxazolidin-5-ylmethyl]-acetamide (Step 1V, XII-5)

The title compound was prepared from N-(2,4-dimethoxy-benzyl)-N-(2-oxo-oxazolidin-5-ylmethyl)-acetamide and 8-bromo-3,4-dihydro-2H-benzo[b]oxepin-5-one (Example 9, step IV) according to the procedure as described in Example 4, step 1V (Yield: 61 mg). The crude product was used without further purification. LC-MS: m/z 469.2 (M+1), 513.2 (M+HCOOH-1).

E. N-[2-Oxo-3-(5-oxo-2,3,4,5-tetrahydro-benzo[b]oxepin-8-yl)-oxazolidin-5-ylmethyl]-acetamide (Step V, XII-6)

The title compound was prepared from N-(2,4-dimethoxy-benzyl)-N-[2-oxo-3-(5-oxo-2,3,4,5-tetrahydro-benzo[b]oxepin-8-yl)-oxazolidin-5-ylmethyl]-acetamide (Example 7, step V) according to the procedure as described in Example 4, step V (Yield: 102 mg, 41%, two steps). H¹NMR (400 MHz, CD₃OD): δ 1.95 (s, 3H), 2.16 (quintet, J=7 Hz, 2H), 2.81 (t, J=7 Hz, 2H), 3.55 (d, J=5 Hz, 2H), 3.79-3.83 (dd, J=6.2, 9 Hz, 1H), 4.14 (t, J=9 Hz, 1H), 4.21 (t, J=6.6 Hz, 2H), 4.744.82 (m, 1H), 7.27-7.33 (m, 2H), 7.69 (d, J=8.3 Hz, 1H). LC-MS: m/z 319.2 (M+1), 363.1 (M+HCOOH-1).

### EXAMPLE 13

### N-[2-Oxo-3-(2,3,4,5-tetrahydro-benzo[f][1,4]oxazepin-7-yl)-oxazolidin-5-ylmethyl]-acetamide hydrochloride (XIII-10)

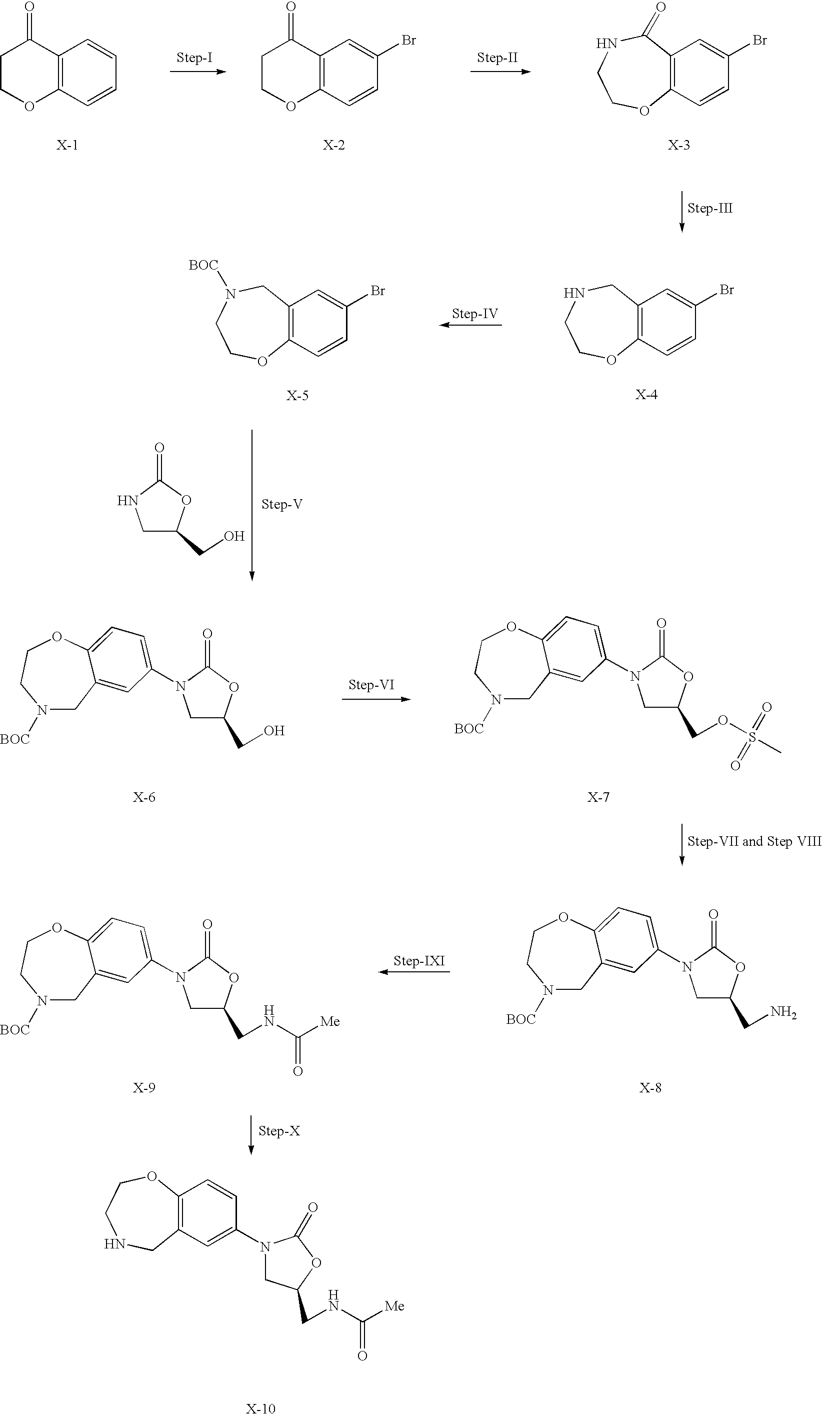

A. 6-Bromo-chroman-4-one (Step I, XIII-2)

To mechanically stirred neat aluminum trichloride (18.0 g, 135 mmol, 2.50 equiv) was added 4-chromanone (8.00 g, 54.0 mmol) portionwise at room temperature. The resulting brown oil was stirred for 10 minutes and bromine (3.34 mL, 65.8 mmol, 1.20 equiv) was added portionwise. The mixture was stirred for 10 minutes, heated to 80° C. for 10 minutes, cooled to 0° C. and quenched with careful addition of ice. The mixture was then diluted with ether and water, the organic layer was washed with 1 M hydrochloric acid (3×), brine, dried over sodium sulfate, filtered and concentrated. The resulting residue was purified on a 40M Biotage column (0 to 10% ethyl acetate in hexanes over 30 minutes) to give 7.61 g of 6-bromo-chroman-4-one (Yield: 62%). The isolated product contains 21% dibromide. H¹NMR (400 MHz, CDCl₃): δ 2.80 (t, 2H), 4.53 (t, 2H), 6.85 (d, 1H), 7.53 (dd, 1H), 7.98 (d, 1H).

B. 7-Bromo-3,4-dihydro-2H-benzo[f][1,4]oxazepin-5-one (Step II, XIII-3)

To a solution of 6-bromo-chroman-4-one (1.35 g, 5.95 mmol) in benzene (20 ml) was added sodium azide (1.55 g, 23.8 mmol, 4 eq) and cooled to 0° C. Concentrated sulfuric acid (4 ml) was added drop wise while maintaining the internal temperature below 5° C. and the reaction mixture was stirred at room temperature overnight. After the benzene layer was carefully decanted, the residue was dissolved in ethyl acetate, washed with water (2×), brine, dried over sodium sulfate and concentrated in vacuo to give (Yield: 1.17 g, 81%) 88:12 mixture of 7-bromo-3,4-dihydro-2H-benzo[f][1,4]oxazepin-5-one and its isomeric amide. The crude mixture was purified on a 40S Biotage (0 to 100% ethyl acetate in hexanes over 30 minutes) to give 0.97 g of the title compound (Yield: 67%). H¹ NMR (400 MHz, CDCl₃): δ 3.50 (q, 2H), 4.38 (t, 2H), 6.75 (broad s, 1H, NH), 6.88 (d, 1H), 7.50 (dd, 1H), 8.12 (d, 1H).

C. 7-Bromo-2,3,4,5-tetrahydro-benzo[f][1,4]oxazepine hydrochloride (Step IV, XIII-4)

To a solution of 7-bromo-3,4-dihydro-2H-benzo[f][1,4]oxazepin-5-one (710 mg, 2.93 mmol) in anhydrous ethylene glycol dimethyl ether (5 mL) under nitrogen was added borane-dimethyl sulfide complex solution (10.0 M, 0.59 mL, 5.87 mmol) and was refluxed overnight. The resulting solution was concentrated in vacuo, dissolved in anhydrous methanol and hydrogen chloride gas was bubbled through for five minutes. The flask was capped and stirred at room temperature for 20 minutes, after which the solution was concentrated in vacuo. The residual solid was triturated with anhydrous ether, filtered and dried under high vacuum to afford the title compound (Yield: 520 mg, 67%). H¹NMR (400 MHz, DMSO-D₆): δ 3.42 (br s, 2H), 4.18 (t, J=4 Hz, 2H), 4.28 (br s, 2H), 7.01(d, J=8.3 Hz, 1H), 7.48 (dd, J=2.5, 8.3 Hz, 1H), 7.67 (d, J=2.5 Hz, 1H), 9.63 (br s, 2H, NH₂). LC-MS (EI): m/z 228.1/230.1 (M+1).

D. 7-Bromo-2,3-dihydro-5H-benzo[f][1,4]oxazepine-4-carboxylic acid tert-butyl ester (Step IV, XIII-5)

To a suspension of 7-bromo-2,3,4,5-tetrahydro-benzo[f][1,4]oxazepine hydrochloride (519 mg, 1.96 mmol) in anhydrous dichloromethane (5 mL) was added diisopropylethylamine (0.75 mL, 4.32 mmol), followed by di-tert-butyl-dicarbonate (471 mg, 2.16 mmol) under nitrogen and stirred at room temperature for 1.5 hours. The solution was diluted with ethyl acetate, washed with 1 M hydrochloric acid (2×), brine, dried over anhydrous sodium sulfate and concentrated in vacuo. The residue was purified on a Biotage 40S column (0 to 100% ethyl acetate in hexanes, 30 minutes) to give the title compound (Yield: 572 mg, 89%). LC-MS (EI): m/z 313.2/315.2 (M—Me), 269.2/271.2 (M-tert-butyl), 228.1/230.1 (M-boc).

E. 7-(5-Hydroxymethyl-2-oxo-oxazolidin-3-yl)-2,3-dihydro-5H-benzo[f][1,4]oxazepine-4-carboxylic acid tert-butyl ester (Step V, XIII-6)

A suspension of 5-hydroxymethyl-oxazolidin-2-one (187 mg, 1.59 mmol), 7-bromo-2,3-dihydro-5H-benzo[f][1,4]oxazepine-4-carboxylic acid tert-butyl ester (523 mg, 1.59 mmol), potassium carbonate (461 mg, 3.34 mmol, 2.10 eq), and racemic trans-1,2-diaminocyclohexane (40 ul, 0.32 mmol, 0.20 eq) in anhydrous dimethylformamide (1.5 ml) was purged four times. Copper(I) iodide (61 mg, 0.32 mmol, 0.20 eq) was added, the mixture was purged with stirring another four times and heated to 105° C. overnight under nitrogen. The reaction mixture was diluted with ethyl acetate and water, the organic layer was washed with 1 M hydrochloric acid (2×), brine, dried over sodium sulfate and concentrated in vacuo to give 7-(5-hydroxymethyl-2-oxo-oxazolidin-3-yl)-2,3-dihydro-5H-benzo[f][1,4]oxazepine-4-carboxylic acid tert-butyl ester (Yield: 563 mg, 97%). The crude product was used in the next step without further purification. H¹NMR (400 MHz, CD₃OD): δ 1.40 (s, 9H), 3.65-4.15 (m, 8H), 4.45 (d, 2H), 4.72 (s, 1H), 6.90-7.0(m, 1H), 7.25-7.65 (m, 2H). LC-MS: m/z 382.3 (M+H₂O), 309.2 (M-tBu), 265.3 (M-boc), 409.3 (M−1+HCOOH).

F. 7-(5-Methanesulfonyloxymethyl-2-oxo-oxazolidin-3-yl)-2,3-dihydro-5H-benzo[f][1,4]oxazepine-4-carboxylic acid tert-butyl ester (Step VI, XIII-7)

To a solution of 7-(5-hydroxymethyl-2-oxo-oxazolidin-3-yl)-2,3-dihydro-5H-benzo[f][1,4]oxazepine-4-carboxylic acid tert-butyl ester (563 mg, 1.55 mmol) in dichloromethane (5 ml) was added diisopropylethylamine (0.30 ml, 1.71 mmol, 1.10 eq) and methanesulfonyl chloride (0.13 ml, 1.62 mmol, 1.05 eq) at room temperature and stirred overnight. The solution was diluted with ethyl acetate, washed with 1 M hydrochloric acid (2×), brine, dried over sodium sulfate and concentrated. The residue was triturated with diethyl ether (2×) and dried in vacuo to give 7-(5-methanesulfonyloxymethyl-2-oxo-oxazolidin-3-yl)-2,3-dihydro-5H-benzo[f][1,4]oxazepine-4-carboxylic acid tert-butyl ester (Yield: 500 mg, 73%). H¹NMR (400 MHz, CD₃OD): δ 1.40 (s, 9H), 3.08 (s, 3H), 3.80 (br s, 2H), 3.95 (t, 1H), 4.02 (m, 2H), 4.15 (m, 1H), 4.40-4.50 (m, 4H), 4.90 (m, 1H), 7.05 (d, 1H), 7.10-7.25 (m, 1H), 7.55(br s, 1H). LC-MS: m/z 460.2 (M+H2O), 387.1 (M−tBu), 343.2 (M-boc).

G. 7-(5-Aminomethyl-2-oxo-oxazolidin-3-yl)-2,3-dihydro-5H-benzo[f][1,4]oxazepine-4-carboxylic acid tert-butyl ester (Step VII and VIII, XIII-8)

To a solution of 7-(5-methanesulfonyloxymethyl-2-oxo-oxazolidin-3-yl)-2,3-dihydro-5H-benzo[f][1,4]oxazepine-4-carboxylic acid tert-butyl ester (500 mg, 1.13 mmol) in dimethylformamide (3 ml) was added sodium azide (808 mg, 1.24 mmol, 1.10 eq) and heated at 80° C. for 2 hours. The solution was cooled to room temperature and diluted with ethyl acetate. The organic phase was washed with water (2×), brine and transferred to a hydrogenation bottle. 10% Palladium on carbon (50 mg) was added and the mixture was hydrogenated at 50 psi for 2.5 hours. The solution was diluted with methanol, filtered through a pad of celite and concentrated in vacuo to give 7-(5-aminomethyl-2-oxo-oxazolidin-3-yl)-2,3-dihydro-5H-benzo[f][1,4]oxazepine-4-carboxylic acid tert-butyl ester (402 mg, 98%, two steps). LC-MS: m/z 308.6 (M−tBu), 264.6 (M-boc).

H. 7-[5-(Acetylamino-methyl)-2-oxo-oxazolidin-3-yl]-2,3-dihydro-5H-benzo[f][1,4]oxazepine-4-carboxylic acid tert-butyl ester (Step VIII, XIII-9)

To a solution of 7-(5-aminomethyl-2-oxo-oxazolidin-3-yl)-2,3-dihydro-5H-benzo[f][1,4]oxazepine-4-carboxylic acid tert-butyl ester (402 mg, 1.11 mmol) in dimethyl formamide (5 ml) was added diisopropylethylamine (0.29 ml, 1.67 mmol, 1.50 eq) and acetyl chloride (0.10 ml, 1.44 mmol, 1.30 eq) at room temperature and stirred for 20 minutes. The solution was diluted with ethyl acetate, washed with 1 M hydrochloric acid (2×), brine, dried over sodium sulfate and concentrated. The residue was dissolved in methanol and filtered through a DOWEX 1×4-100 ion exchange resin plug (strongly basic anion, 4% cross-linking) and concentrated in vacuo to give 7-[5-(acetylamino-methyl)-2-oxo-oxazolidin-3-yl]-2,3-dihydro-5H-benzo[f][1,4]oxazepine-4-carboxylic acid tert-butyl ester (Yield: 268 mg, 60%). H¹NMR (400 MHz, CD₃OD), rotamers: δ 1.40 (s, 9H), 3.55 (d, 1H), 3.70-3.8 (m, 3H), 4.0 (apparent s, 2H), 4.15 (m, 1H), 4.40-4.43 (s, 2H), 4.78 (m, 1H), 7.0-7.8 (br m, 3H). LC-MS: m/z 350.5 (M−tBu), 306.5 (M-boc).

I. N-[2-Oxo-3-(2,3,4,5-tetrahydro-benzo[f][1,4]oxazepin-7-yl)-oxazolidin-5-ylmethyl]-acetamide hydrochloride (Step IX, XIIII-10)

A solution of 7-[5-(acetylamino-methyl)-2-oxo-oxazolidin-3-yl]-2,3-dihydro-5H-benzo[f][1,4]oxazepine-4-carboxylic acid tert-butyl ester (234 mg, 0.58 mmol) in 4 N hydrochloric acid in dioxane was stirred at room temperature under nitrogen for 20 minutes. The solvent was decanted and the residue was dried under high vacuum to give N-[2-oxo-3-(2,3,4,5-tetrahydro-benzo[f][1,4]oxazepin-7-yl)-oxazolidin-5-ylmethyl]-acetamide hydrochloride (Yield: 183 mg, 92%). H¹NMR (500 MHz, DMSO-d₆): δ 1.84 (s, 3H), 3.4-3.5 (m, 4H), 3.74 (t, 1H), 4.10-4.22 (m, 3H), 4.34 (broad s, 2H), 4.75 (m, 1H), 7.13 (d, 1H), 7.52 (d, 1H), 7.62 (s, 1H), 8.32 (t, 1H, NH), 9.43 (br s, 2H, NH₂).

### EXAMPLE 14

### Preparation of N-[2-Oxo-3-(2,3,4,5-tetrahydro-1H-benzo[b]azepin-7-yl)-oxazolidin-5-ylmethyl]-acetamide (XIV-12)

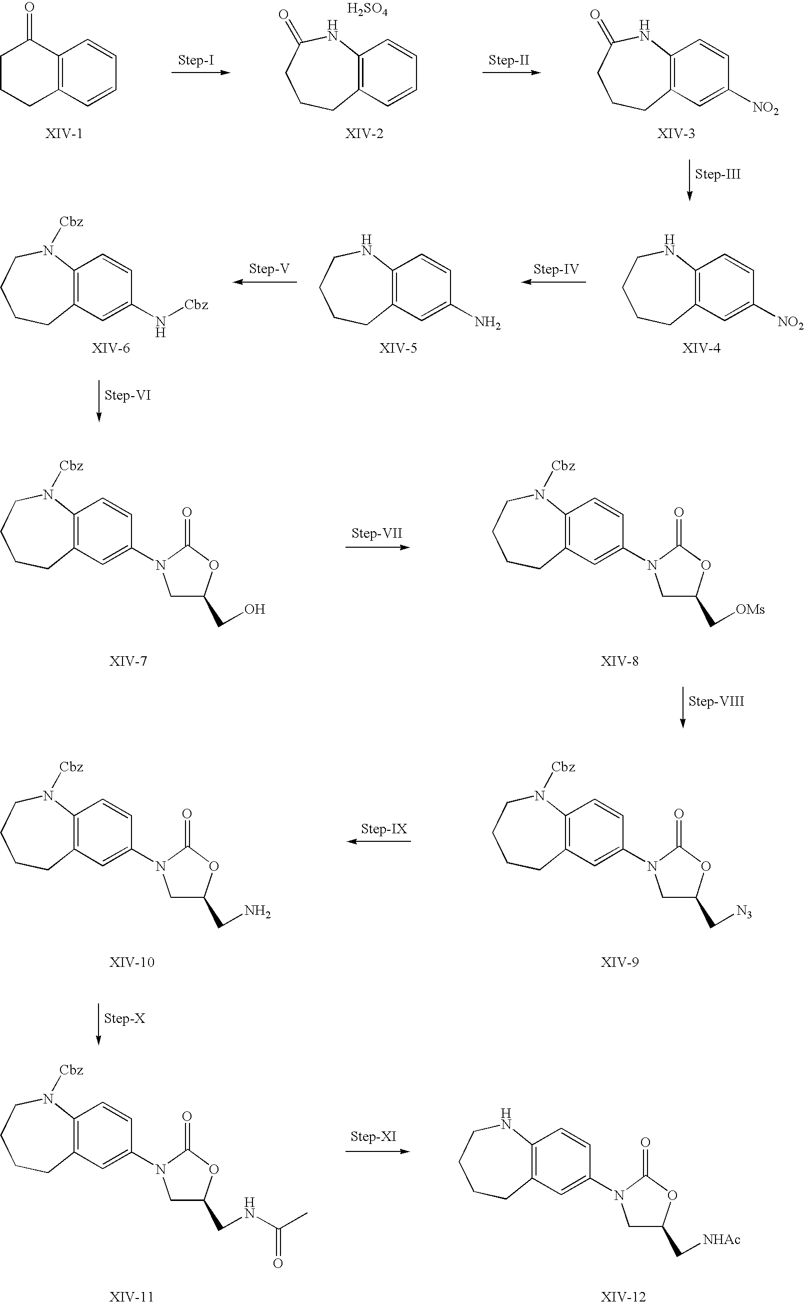

A. 1,3,4,5-Tetrahydro-benzo[b]azepin-2-one sulfate (Step-I, XIV-2)

To a solution of tetralone (7.60 g, 52.0 mmol) in benzene (100 ml) was added sodium azide (13.52 g, 208 mmol, 4.00 eq) and cooled to 0° C. Concentrated sulfuric acid (12 ml) was added dropwise while maintaining the internal temperature below 5° C. and the reaction mixture was stirred at room temperature for 4 hours. After the benzene layer was carefully decanted, the residue was triturated with ether. The solid that crushed out was filtered and washed repeatedly with 1:1 ethyl acetate/methanol. The ethyl acetate methanol layer was concentrated in vacuo to afford 1,3,4,5-tetrahydro-benzo[b]azepin-2-one sulfate (Yield: 6.61 g, 49%). H¹NMR (400 MHz, DMSO-d₆): δ 2.03 (m, 4H), 2.59 (t, 2H), 6.85 (d, 1H), 7.00 (t, 1H), 7.15 (m, 2H), 8.4-8.7 (br s, 2H), 9.42 (s, 1H, NH).

B. 7-Nitro-1,3,4,5-tetrahydro-benzo[b]azepin-2-one (Step II, XIV-3)

Concentrated nitric acid (0.95 ml) and concentrated sulfuric acid (10 ml) were mixed and cooled to 0-5° C. To a flask that was charged with 1,3,4,5-tetrahydro-benzo[b]azepin-2-one sulfate was added the nitric acid/sulfuric acid mixture drop wise over 10 minutes. The resulting mixture was stirred for 20 minutes at 5° C. and poured over ice. The solid that crushed out was filtered, rinsed with water, ether and dried under high vacuum to give 7-nitro-1,3,4,5-tetrahydro-benzo[b]azepin-2-one (Yield: 2.68 g, 82%). H¹NMR (400 MHz, CDCl₃): δ 2.28 (q, 2H), 2.42 (t, 2H), 2.90 (t, 2H), 7.05 (d, 1H), 8.12-8.15 (m, 2H). The product contains approximately 16% of a minor isomer.

C. 7-Nitro-2,3,4,5-tetrahydro-1H-benzo[b] azepine hydrochloride (Step III, XIV-4)

To a solution of 7-nitro-1,3,4,5-tetrahydro-benzo[b]azepin-2-one (3, 2.59 g, 12.6 mmol) in THF (10 ml) was added borane-dimethyl sulfide complex (10.0 M, 3.14 ml, 31.4 mmol, 2.5 eq) and the solution was heated to 45° C. under nitrogen overnight. The excess borane was quenched by slow addition of methanol. Hydrogen chloride gas was bubbled through the solution for 5 minutes and the solution was concentrated in vacuo to give 7-nitro-2,3,4,5-tetrahydro-1H-benzo[b]azepine hydrochloride (Yield: 2.61 g, 91%). H¹NMR (400 MHz, DMSO-d₆): δ 1.78 (m, 4H), 2.83 (m, 2H), 3.23 (m, 2H), 6.75 (d, 1H), 7.79 (dd, 1H), 7.82 (d, 1H). LC-MS: m/z 193.2 (M+1), 237.2 (M−1+HCOOH).

D. 2,3,4,5-Tetrahydro-1H-benzo[b]azepin-7-ylamine (Step 1V, XIV-5)

A mixture of 7-nitro-2,3,4,5-tetrahydro-1H-benzo[b]azepine hydrochloride (IX-4, 2.50 g, 8.55 mmol) in methanol (30 ml) and 10% palladium on carbon (200 mg) was hydrogenated at 45 psi overnight at room temperature. The reaction mixture was filtered through a pad of celite and concentrated to give 2,3,4,5-tetrahydro-1H-benzo[b]azepin-7-ylamine (Yield: 2.22 g, 99%). The crude product was used in the next step without any further purification. H¹NMR (400 MHz, CDCl₃): δ 1.60 (m, 2H), 1.82 (m, 2H), 2.85 (t, 2H), 3.02 (t, 2H), 6.41 (dd, 1H), 6.49 (d, 1H), 6.70 (d, 1H).

E. 7-Benzyloxycarbonylamino-2,3,4,5-tetrahydro-benzo[1]azepine-1-carboxylic acid benzyl ester (Step IV, XIV-6)

To a solution of 2,3,4,5-tetrahydro-1H-benzo[b]azepin-7-ylamine (2.17 g, 13.4 mmol) in dichloromethane (15 ml) was added diisopropylethylamine (9.6 ml, 54.9 mmol, 4.10 eq) and benzyloxycarbonyl chloride (3.82 ml, 26.8 mmol, 2.00 eq) at room temperature and stirred for 1.5 hours. The solution was diluted with ethyl acetate, washed with 1 M hydrochloric acid (2×), brine, dried over sodium sulfate, and concentrated in vacuo. The residue was purified on a 40M Biotage column (0 to 100% ethyl acetate in hexanes over 30 minutes) to give 7-benzyloxycarbonylamino-2,3,4,5-tetrahydro-benzo[b]azepine-1-carboxylic acid benzyl ester (3.93 g, 68%). LC-MS: m/z 431.3 (M+1), 429.3 (M−1).

F. 7-(5-Hydroxymethyl-2-oxo-oxazolidin-0.3-yl)-2,3,4,5-tetrahydro-benzo[b]azepine-1-carboxylic acid benzyl ester (Step VI, XIV-7)

To a flame dried flask charged with 7-benzyloxycarbonylamino-2,3,4,5-tetrahydro-benzo[b]azepine-1-carboxylic acid benzyl ester (3.93 g, 9.13 mmol) and THF (30.0 mL) was added n-Butyl Lithium (4.0 mL, 2.5 M in hexanes) drop wise at −78° C. The reaction stirred at −78° C. for 30 minutes after which R-glycidol butyrate (1.36 mL, 9.59 mmol) was added. The reaction warmed to room temperature and stirred over 72 hours. Diluting with saturated solution of ammonium chloride subsequently quenched the reaction. The aqueous mixture was extracted with ethyl acetate 3× and the combined organic fractions were washed with brine, dried over sodium sulfate, and concentrated. Purification using silica gel chromatography using gave the title compound (Yield: 100 g, 22%) along with starting material (1.89 g, 52%). MS (CI) m/z 397 (M+H).

G. 7-(5-Methanesulfonyloxymethyl-2-oxo-oxazolidin-3-yl)-2,3,4,5-tetrahydro-benzo[b]azepine-1-carboxylic acid benzyl ester (Step VII, XIV-8)

To a solution of 7-(5-hydroxymethyl-2-oxo-oxazolidin-3-yl)-2,3,4,5-tetrahydro-benzo[b]azepine-1-carboxylic acid benzyl ester (1.89 g, 0.36 mmol) in methylene chloride (20.0 mL) cooled to 0° C. was added diisopropylethyl amine (1.04 mL, 5.96 mmol) followed by mesyl chloride (0.41 mL, 5.24 mmol). The reaction warmed to room temperature and continued to stir for 16 hours after which the reaction was diluted with ethyl acetate. The mixture was washed with water 3×, 1N hydrochloric acid, brine, dried over sodium sulfate, and concentrated to give the title compound (Yield: 2.04 g). MS (CI) m/z 475 (M+H). The crude product was used directly in the next reaction without further purification.

H. 7-(5-Azidomethyl-2-oxo-oxazolidin-3-yl)-2,3,4,5-tetrahydro-benzo[b]azepine-1-carboxylic acid benzyl ester (Step VIII, XIV-9)

To a solution of 7-(5-methanesulfonyloxymethyl-2-oxo-oxazolidin-3-yl)-2,3,4,5-tetrahydro-benzo[b]azepine-1-carboxylic acid benzyl ester (2.04 g crude material from the previous step, 4.30 mmol) in DMF (10.0 mL) was added sodium azide (0.56 g, 8.6 mmol) and the reaction heated to 80° C. for 180 minutes. The reaction was diluted with ethyl acetate and washed with water 4×, brine, and dried over sodium sulfate and concentrated to the title compound (1.78 g, 98% yield). MS (CI) m/z 422 (M+H). The crude product was used directly in the next step without further purification.

I. 7-(5-Aminomethyl-2-oxo-oxazolidin-3-yl)-2,3,4,5-tetrahydro-benzo[b]azepine-1-carboxylic acid benzyl ester (Step IX, XIV-10)

To a solution of 7-(5-Azidomethyl-2-oxo-oxazolidin-3-yl)-2,3,4,5-tetrahydro-benzo[b]azepine-1-carboxylic acid benzyl ester (IX-9, 1.78 g crude material from the previous step, 4.22 mmol) in ethyl acetate (15 mL) was added 10% Pd/C (150 mg). The reaction was hydrogenated at 35 psi for 5 hours and then filtered through celite. The solvent was evaporated to give the title compound (Yield: 1.60 g). MS (CI) m/z 396 (M+H, 96% yield). The crude product was used directly in the next step without further purification.

J. 7-[5-(Acetylamino-methyl)-2-oxo-oxazolidin-3-yl]-2,3,4,5-tetrahydro-benzo[b]azepine-1-carboxylic acid benzyl ester (Step X, XIV-11)

To a flame dried flask charged with 7-(5-Aminomethyl-2-oxo-oxazolidin-3-yl)-2,3,4,5-tetrahydro-benzo[b]azepine-1-carboxylic acid benzyl ester (1.6 g crude material from the previous step, ˜4.05 mmol), and diisopropylethylamine (1.06 mL, 6.08 mmol) in methylene chloride (10.0 mL) at RT, was added acetic anhydride (0.4 mL, 4.25 mmol). The reaction was stirred for 3 hours. The reaction mixture was diluted with dichloromethane, washed with 1 N hydrochloric acid, brine and dried over sodium sulfate. MS (CI) m/z 438 (M+H), (1.68 g, 95% yield).

K. N-[2-Oxo-3-(2,3,4,5-tetrahydro-1H-benzo[b]azepin-7-yl)-oxazolidin-5-ylmethyl]-acetamide (Step XI, XIV-12)

7-[5-(Acetylamino-methyl)-2-oxo-oxazolidin-3-yl]-2,3,4,5-tetrahydro-benzo[b]azepine-1-carboxylic acid benzyl ester (1.59 g, 3.63 mmol) was taken in 15 mL of methanol. To it palladium hydroxide (100 mg) was added and kept under shaking under hydrogen pressure (50 psi). The catalyst was removed by filtering through celite and washed with methanol. The solvent was removed and purifed by silica gel chromatography. MS (CI) m/z 304 (M+H), (0.91 g, 83% yield).

### EXAMPLE 15

### N-[2-Oxo-3-(2,3,4,5-tetrahydro-1H-benzo [c] azepin-7-yl)-oxazolidin-5-ylmethyl]-acetamide and N-[2-Oxo-3-(2,3,4,5-tetrahydro-1H-benzo[d]azepin-7-yl)-oxazolidin-5-ylmethyl]-acetamide (X-10A and X-10B)

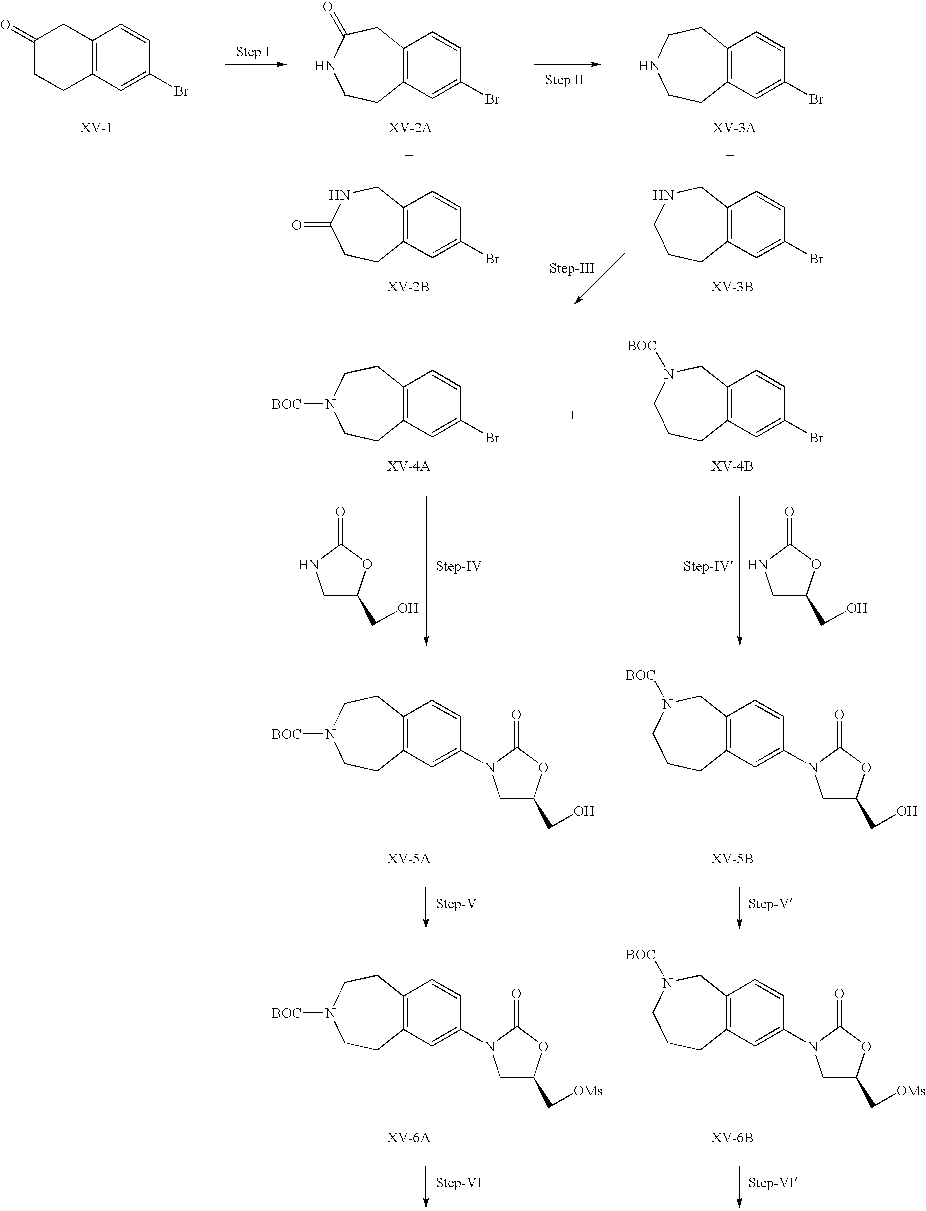

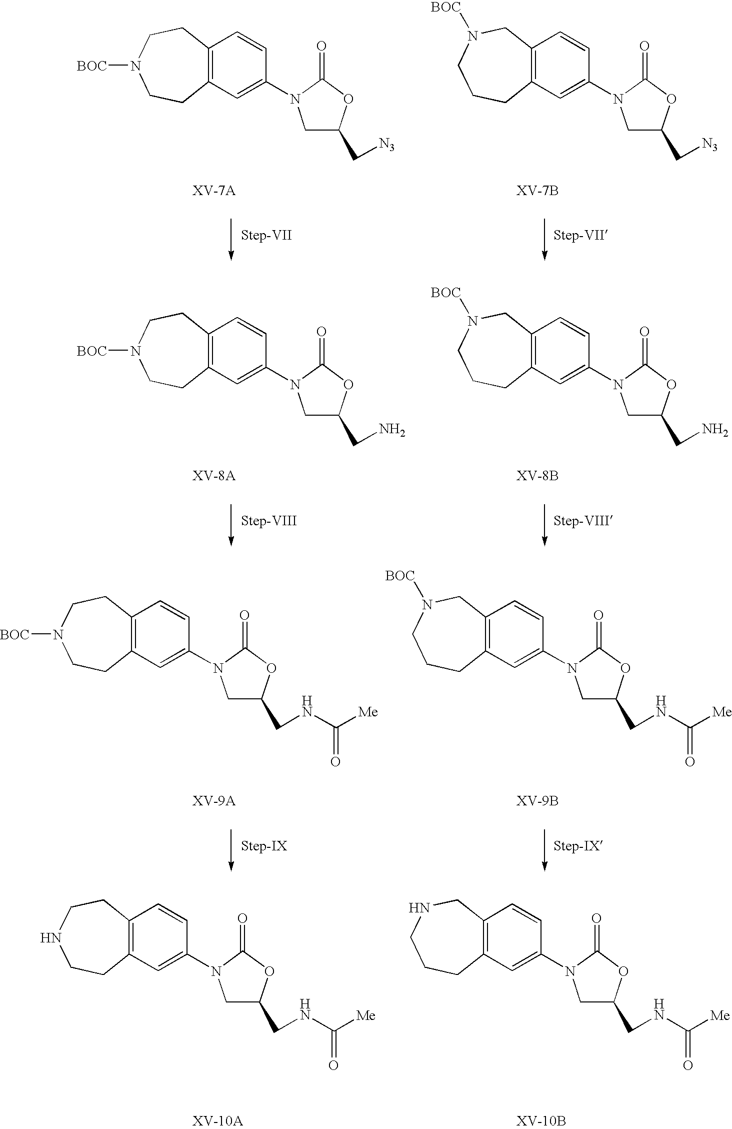

A. 7-Bromo-1,3,4,5-tetrahydro-benzo[d]azepin-2-one and 7-Bromo-1,2,4,5-tetrahydro-benzo[c]azepin-3-one (Step I, XV-2A and 2B)

6-Bromo-3,4-dihydro-1H-naphthalen-2-one (2.0 g, 8.89 mmol) was dissolved in benzene and cooled to 0° C. To it sodium azide (2.31 g, 35.5 mmol) was added followed by dropwise addition of concentration sulfuric acid (4 mL). The ice bath was removed and the reaction mixture was stirred at room temperature overnight. The reaction was diluted with ethyl acetate, washed with water (2×) and brine; dried over sodium sulfate. The solvents were evaporated to obtain the title compounds as mixture and are separable by crystallization with ethyl acetate. LC-MS m/z 240 and 242 (M+H) (Yield: 1.9 g). However, the mixture of regioisomers was taken together into the next step.

B. 7-Bromo-2,3,4,5-tetrahydro-1H-benzo[d]azepine and 7-Bromo-2,3,4,5-tetrahydro-1H-benzo[c]azepine (Step II, XV-3A and 3B)

The title compounds were prepared from the mixture of 7-Bromo-1,3,4,5-tetrahydro-benzo[d]azepin-2-one and 7-Bromo-1,2,4,5-tetrahydro-benzo[c]azepin-3-one via borane reduction as described in Example 13, step III. LC-MS: m/z 228/330.

C. 7-Bromo-2,3,4,5-tetrahydro-1H-benzo [d]azepine-2-carboxylic acid tert-butyl ester and 7-Bromo-2,3,4,5-tetrahydro-1H-benzo [c] azepine-2-carboxylic acid tert-butyl ester (Step III, XV 4A and 4B)

The title compounds were prepared from 7-Bromo-2,3,4,5-tetrahydro-1H-benzo[d]azepine and 7-Bromo-2,3,4,5-tetrahydro-1H-benzo[c]azepine using Boc anhydride as described in Example 13, Step IV. The crude product was subjected to flash chromatography, resulted in the separation of the title regioisomers, A and B. (42% isolated yield, overall 3 steps). LC-MS: m/z 311.7/313.7 (M-Me), 270.6/272.6 (M−tBu), 228.1/230.1 (M-boc).

D(i) 7-(5-Hyd roxymethyl-2-oxo-oxazolidin-3-yl)-1,3,4,5-tetrahydro-1H-benzo[d]azepine-2-carboxylic acid tert-butyl ester (Step IV, XV-5A)

The title compound was prepared from 7-Bromo-2,3,4,5-tetrahydro-1H-benzo[d]azepine-2-carboxylic acid tert-butyl ester as described in Example 13, step V. The crude product was purified by flash silica gel chromatography to yield the title compound. Yield: 89%. LC-MS: m/z 308 (M+18).

D(ii) 7-(5-Hydroxymethyl-2-oxo-oxazolidin-3-yl)-1,3,4,5-tetrahydro-1H-benzo[c]azepine-2-carboxylic acid tert-butyl ester (Step IV, XV-5B)

The title compound was prepared from 7-Bromo-2,3,4,5-tetrahydro-1H-benzo[c]azepine-2-carboxylic acid tert-butyl ester as described in Example 13, step V. Yield: 64%. LC-MS: m/z 308 (M+18).

E(i) 7-(5-Methanesulfonyloxymethyl-2-oxo-oxazolid in-3-yl)-1,3,4,5-tetrahydro-1H-benzo[d]azepine-2-carboxylic acid tert-butyl ester (Step V, XV-6A)

The title compound was prepared from 7-(5-hydroxymethyl-2-oxo-oxazolidin-3-yl)-1,3,4,5-tetrahydro-1H-benzo[d]azepine-2-carboxylic acid tert-butyl ester according to the procedure as Example 13, step VI. (Yield: 93%). LC-MS: m/z 458.3.

E(ii) 7-(5-Methanesulfonyloxymethyl-2-oxo-oxazolidin-3-yl)-1,3,4,5-tetrahydro-1H-benzo[c]azepine-2-carboxylic acid tert-butyl ester (Step V, XV-6B)

The title compound was prepared from 7-(5-hydroxymethyl-2-oxo-oxazol idin-3-yl)-1,3,4,5-tetrahydro-1H-benzo[c]azepine-2-carboxylic acid tert-butyl ester according to the procedure as Example 13, step VI. (Yield: 94%). H¹NMR (400 MHz, CDCl₃): δ1.38 (s, 9H), 1.75 (m, 2H), 2.93 (m, 2H), 3.63 (broad s, 2H), 3.92 (t, 1H), 4.12 (m, 1H), 4.32 (broad s, 2H), 4.38-4.49 (qd, 2H), 4.87 (m, 1H), 7.05-7.5 (broad m, 3H). LC-MS: m/z 458.3.

F(i) 7-(5-Azidomethyl-2-oxo-oxazolidin-3-yl)-1,3,4,5-tetrahydro-1H-benzo[d]azepine-2-carboxylic acid tert-butyl ester (Step VI, XV-7A)

The title compound was prepared from 7-(5-methanesulfonyloxymethyl-2-oxo-oxazolidin-3-yl)-1,3,4,5-tetrahydro-1H-benzo[d]azepine-2-carboxylic acid tert-butyl ester according to the same procedure as Example 13, step VII. (Yield: 163 mg, 95%). LC-MS: m/z 405.2.

F(ii) 7-(5-Azidomethyl-2-oxo-oxazolidin-3-yl)-1,3,4,5-tetrahydro-1H-benzo[c]azepine-2-carboxylic acid tert-butyl ester (Step VI, XV-7B)

The title compound was prepared from 7-(5-methanesulfonyloxymethyl-2-oxo-oxazolidin-3-yl)-1,3,4,5-tetrahydro-1H-benzo[c]azepine-2-carboxylic acid tert-butyl ester according to the procedure as Example 13, step-VII. (Yield: 163 mg, 94%). H¹NMR (400 MHz, CDCl₃): δ 1.40 (s, 9H), 1.75 (m, 2H), 2.93 (m, 2H), 3.58 (dd, 1H), 3.68 (m, 2H), 3.85 (m, 1H), 4.18 (t, 1H), 4.34 (broad s, 2H), 4.75 (m, 1H), 7.05-7.4 (broad m, 3H). LC-MS: m/z 405.2 (M+1), 432.2 (M-I+HCOOH).

G(i). 7-(5-Aminomethyl-2-oxo-oxazolidin-3-yl)-1,3,4,5-tetrahydro-benzo[d]azepine-2-carboxylic acid tert-butyl ester (Step VII, XV-8A)

7-(5-Azidomethyl-2-oxo-oxazolidin-3-yl)-1,3,4,5-tetrahydro-1H-benzo[d]azepine-2-carboxylic acid tert-butyl ester (505 mg, 0.42 mmol) was taken in 5 mL of methanol. To it palladium hydroxide (50 mg) was added and shaken under hydrogen atmosphere at 45 psi pressure. After 16 hours, the reaction mixture was diluted with ethyl acetate and filtered through a pad of celite. The solvents were evaporated to give the title compound. (Yield: 141 mg, 99%). LC-MS: m/z 262.6.

G(ii). 7-(5-Aminomethyl-2-oxo-oxazolidin-3-yl)-1,3,4,5-tetrahydro-benzo[c]azepine-2-carboxylic acid tert-butyl ester (Step VII, XV-8B) 7-(5-Azidomethyl-2-oxo-oxazolidin-3-yl)-1,3,4,5-tetrahydro-1H-benzo[c]azepine-2-carboxylic acid tert-butyl ester (163 mg, 0.42 mmol) was taken in 5 mL of methanol. To it palladium hydroxide (20 mg) was added and shaken under hydrogen atmosphere at 45 psi pressure. After 16 hours, the reaction mixture was diluted with ethyl acetate and filtered through a pad of celite. The solvents were evaporated to give the title compound. (Yield: 141 mg, 93%). LC-MS: m/z 262.6 (M−Boc).

H(i). 7-[5-(Acetylamino-methyl)-2-oxo-oxazolidin-3-yl]-1,3,4,5-tetrahydro-benzo[d]azepine-2-carboxylic acid tert-butyl ester (Step VIII, XV-9B)

7-(5-Aminomethyl-2-oxo-oxazolidin-3-yl)-1,3,4,5-tetrahydro-benzo[d]azepine-2-carboxylic acid tert-butyl ester (470 mg, 1.3 mmol) was taken in 5 mL of dichloromethane. To it diisopropylethylamine (0.28 mL, 1.63 mmol), followed by acetyl chloride (102 □L, 1.43 mmol) was added. The reaction was stirred at room temperature for 20 minutes. The reaction was diluted with ethyl acetate; washed with 1 N hydrochloric acid, brine; dried over sodium sulfate. The solvents were evaporated to obtain the title compound. (Yield: 150 mg, 88% yield). LC-MS: m/z 304.7 (M−Boc).

H(ii). 7-[5-(Acetylamino-methyl)-2-oxo-oxazolidin-3-yl]-1,3,4,5-tetrahydro-benzo[c]azepine-2-carboxylic acid tert-butyl ester (Step VIII, XV-9B)

7-(5-Aminomethyl-2-oxo-oxazolidin-3-yl)-1,3,4,5-tetrahydro-benzo[c]azepine-2-carboxylic acid tert-butyl ester (141 mg, 0.39 mmol) was taken in 3 mL of dichloromethane. To it diisopropylethylamine (0.1 mL, 0.55 mmol), followed by acetyl chloride (33 λL, 0.47 mmol) was added. The reaction was stirred at room temperature for 20 minutes. The reaction was diluted with ethyl acetate; washed with 1 N hydrochloric acid, brine; dried over sodium sulfate. The solvents were evaporated to obtain the title compound. (Yield: 150 mg, 96% yield). LC-MS: m/z 304.7 (M−Boc).

I(i). N-[2-Oxo-3-(2,3,4,5-tetrahydro-1H-benzo [d] azepin-7-yl)-oxazolidin-5-ylmethyl]-acetamide (Step IX, XV-10A)

Deprotection of Boc group was done as described in Example 13, step 1×using 7-[5-(Acetylamino-methyl)-2-oxo-oxazol idin-3-yl]-1,3,4,5-tetrahydro-benzo[d]azepine-2-carboxylic acid tert-butyl ester and 4 N hydrochloric acid. LC-MS: m/z 305 (M+H).

I(ii). N-[2-Oxo-3-(2,3,4,5-tetrahydro-1H-benzo [c] azepin-7-yl)-oxazolidin-5-ylmethyl]-acetamide (Step 1×, XV-10B)

Deprotection of Boc group was done as described in Example 13, step IX using 7-[5-(Acetylamino-methyl)-2-oxo-oxazolidin-3-yl]-1,3,4,5-tetrahydro-benzo[c]azepine-2-carboxylic acid tert-butyl ester and 4N hydrochloric acid. LC-MS: m/z 305 (M+H).

The following illustrates representative pharmaceutical dosage forms, containing a compound of Formula I (“Invention Compound”), for therapeutic or prophylactic use in humans.

| (i) Tablet | mg/tablet |
|---|---|
| ‘Invention Compound’ | 10-1000 |
| Lactose | 50.0 |
| Corn Starch (for mix) | 10.0 |
| Corn Starch (paste) | 10.0 |
| Magnesium Stearate (1%) | 3.0 |

The invention compound, lactose, and corn starch (for mix) are blended to uniformity. The corn starch (for paste) is suspended in 200 mL of water and heated with stirring to form a paste. The paste is used to granulate the mixed powders. The wet granules are passed through a No. 8 hand screen and dried at 80° C. The dry granules are lubricated with the 1% magnesium stearate and pressed into a tablet. Such tablets can be administered to a human from one to four times a day for treatment of pathogenic bacterial infections.

| (ii) Tablet | mg/capsule |
|---|---|
| ‘Invention Compound | 10-1000 |
| Colloidal Silicon Dioxide | 1.5 |
| Lactose | 465.5 |
| Pregelatinized Starch | 120.0 |
| Magnesium Stearate (1%) | 3.0 |

| (iii) Preparation for | |
|---|---|
| Oral Solution | Amount |
| ‘Invention Compound’ | 10-1000 |
| Sorbitol Solution (70% N.F.) | 40 mL |
| Sodium Benzoate | 20 mg |
| Saccharin | 5 mg |
| Cherry Flavor | 20 mg |
| Distilled Water q.s. | 100 mL |

The sorbitol solution is added to 40 mL of distilled water, and the invention compound is dissolved therein. The saccharin, sodium benzoate, flavor, and dye are added and dissolved. The volume is adjusted to 100 mL with distilled water. Each milliliter of syrup contains 4 mg of invention compound.

(iv) Parenteral Solution

In a solution of 700 mL of propylene glycol and 200 mL of water for injection is suspended 20 g of an invention compound. After suspension is complete, the pH is adjusted to 6.5 with 1 N hydrochloric acid, and the volume is made up to 1000 mL with water for injection. The Formulation is sterilized, filled into 5.0 mL ampoules each containing 2.0 mL, and sealed under nitrogen.

| (v) Injection 1 (1 mg/mL) | Amount |
|---|---|
| ‘Invention Compound’ | 10-1000 |
| Dibasic Sodium Phosphate | 12.0 |
| Monobasic Sodium Phosphate | 0.7 |
| Sodium Chloride | 4.5 |
| 1.0 N Sodium hydroxide solution | q.s. |
| (pH adjustment to 7.0-7.5) | |
| Water for injection | q.s. ad 1 mL |

| (vi) Injection 2 (10 mg/mL) | Amount |
|---|---|
| ‘Invention Compound’ | 10-1000 |
| Dibasic Sodium Phosphate | 1.1 |
| Monobasic Sodium Phosphate | 0.3 |
| Polyethylene glyco 400 | 200.0 |
| 0.1 N hydrochloric acid solution | q.s. |
| (pH adjustment to 7.0-7.5) | |
| Water for injection | q.s. ad 1 mL |

| (vii) Injection 2 (10 mg/mL) | Amount |
|---|---|
| ‘Invention Compound’ | 10-1000 |
| Oleic Acid | 10.0 |
| Trichloromonofluoromethane | 5,000.0 |
| Dichlorodifluoromethane | 10,000.0 |
| Dichlorotetrafluoroethane | 5,000.0. |

All patents, and patent documents are incorporated by reference herein, as though individually incorporated by reference. The invention and the manner and process of making and using it, are now described in such full, clear, concise and exact terms as to enable any person skilled in the art to which it pertains, to make and use the same. It is to be understood that the foregoing describes preferred embodiments of the present invention and that modifications may be made therein without departing from the spirit or scope of the present invention as set forth in the claims. To particularly point out and distinctly claim the subject matter regarded as invention, the following claims conclude this specification.

What is claimed is:

1. A compound of formula I: 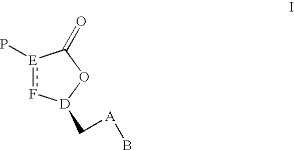
or a pharmaceutically acceptable salt thereof, wherein:
A is O,
NH, or
S;
B is
C(═O)R₁,
C(═S)R₁,
heterocylco,
heteroaryl,
C(═O)-heterocyclo,
C(═N)—CN, or
C(═O)-heteteroaryl;
either D is N, E is C, and F is CH when “------” is a bond, or D is CH, E is N, and F is CH₂ when “------” is absent; 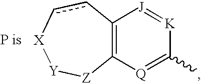 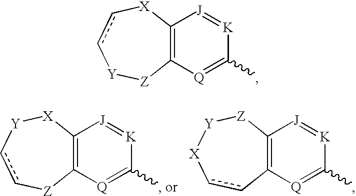
wherein “1” indicates the point of attachment;
J, K, Q independently are CR₂ or N, with the proviso that when any one of J, K, or Q is N, then the other two are CR₂;
“------” is absent; or is a bond; and
X, Y, Z independently are C═C—R₅,
O═C,
CH₂,
CHR₃,
CHR₄,
CR₃R₄,
NR₅,
N(C═O)R₅,
N(C═O)OR₅,
NSO₂R₅,
NSO₂NR₅,
O,
S,
SO, or
SO₂;
R₁ is H,
(C₁-C₈)alkyl,
(C₃-C₆)cycloalkyl,
O—(C₁-C₄)alkyl,
O—(C₃-C₆)cycloalkyl,
S—(C₁-C₄) alkyl,
S—(C₃-C₆)cycloalkyl,
NH₂,
NH(C₁-C₄)alkyl,
N((C₁-C₄)alkyl)₂, or
NH—(C₃-C₆)cycloalkyl;
R₂ is H,
halo,
(C₁-C₈)alkyl,
(C₃-C₆)cycloalkyl,
O—C₁-C₄)alkyl,
O—C₃-C₆)cycloalkyl,
S—C₁-C₄)alkyl,
S—(C₃-C₆)cycloalkyl,
NH₂,
NH(C₁-C₄)alkyl,
N((C₁-C₄)alkyl)₂, or
NH—(C₃-C₆)cycloalkyl;
R₃ and R₄ independently are halo,
(C₁-C₈)alkyl,
(C₃-C₆)cycloalkyl,
O—(C₁-C₄)alkyl,
O—(C₃-C₆)cycloalkyl,
S—(C₁-C₄) alkyl,
S—(C₃-C₆)cycloalkyl,
NH₂,
NH(C₁-C₄)alkyl,
N((C₁-C₄)alkyl)₂,
NH—(C₃-C₆)cycloalkyl;
aryl,
(CH₂)ₙ-aryl,
heterocyclo,
(CH₂)ₙ-heterocyclo,
heteroaryl, or
(CH₂)ₙ-heteroaryl,
wherein n is 0, 1, 2, or 3;
R₅ is H,
(C₁-C₈)alkyl,
(C₃-C₆)cycloalkyl,
aryl,
(CH₂)ₙ-aryl,
heterocyclo,
(CH₂)ₙ-heterocyclo,
heteroaryl, or
(CH₂)ₙ-heteroaryl,
wherein n is as defined above.

2. The compound of claim 1 as designated in formula IA.

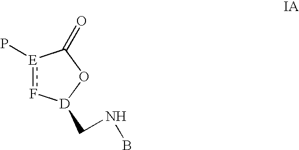

3. The compound of claim 1 as designated in formula IB.

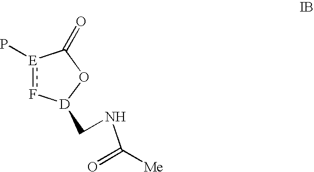

4. The compound of claim 1 as designated in formula IC,

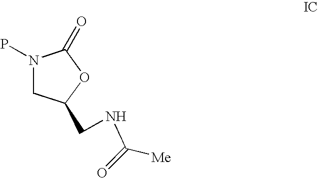

5. The compound of claim 1, wherein P is 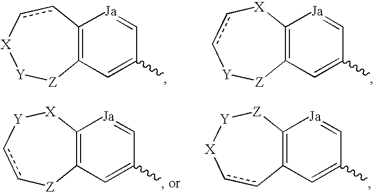
wherein “ [symbol] ”indicates the point of attachment, Jₐ is N or CR′, wherein R′ is H or F

6. The compound of claim 1, wherein P is

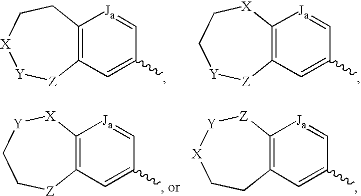

7. The compound of claim 6, wherein two of X, Y, or Z is C═C—R₅,
O═C,
NR₅,
N(C═O)R₅,
N(C═O)OR₅,
NSO₂R₅,
NSO₂NR₅,
O,
S,
SO, or
SO₂NR₅,
and the other of X, Y, or Z is CH₂ or CR₃R₄.

8. The compound of claim 7, wherein P is

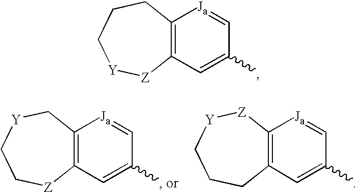

9. The compound of claim 7, wherein P is

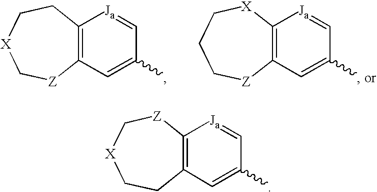

10. The compound of claim 7, wherein P is

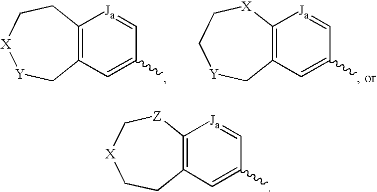

11. The compound of claim 6, wherein one of X, Y, or Z is C═C—R₅,
O═C,
NR₅,
N(C═O)R₅,
N(C═O)OR₅,
NSO₂R₅,
NSO₂NR₅,
O,
S,
SO, or
SO₂NR₅,
and the other of X, Y, or Z is CH₂.

12. The compound of claim 11, wherein P is

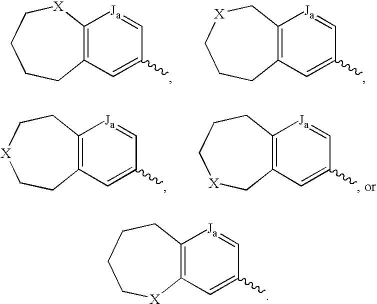

13. A compound of formula II 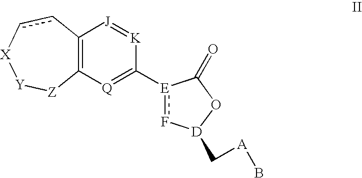
or a pharmaceutically acceptable salt thereof, wherein:
A is O,
NH, or
S;
B is
C(═O)R₁,
C(═S)R₁,
heterocylco,
heteroaryl,
C(═O)-heterocyclo,
C(═N)—CN, or
C(═O)-heteteroaryl;
either D is N, E is C, and F is CH when “------” is a bond, or D is CH, E is N, and F is CH₂ when “------” is absent;
J, K, Q independently are CR₂ or N, with the proviso that when any one of J, K, or Q is N, then the other two are CR₂;
“------” is absent; or is a bond; and
X, Y, Z independently are C═C—R₅,
O═C,
CH₂,
CHR₃,
CHR₄,
CR₃R₄,
NR₅,
N(C═O)R₅,
N(C═O)OR₅,
NSO₂R₅,
NSO₂NR₅,
O,
S,
SO, or
SO₂;
R₁ is H,
(C₁-C₈)alkyl,
(C₃-C₆)cycloalkyl,
O—(C₁-C₄)alkyl,
O—(C₃-C₆)cycloalkyl,
S—(C₁-C₄) alkyl,
S—(C₃-C₆)cycloalkyl,
NH₂,
NH(C₁-C₄)alkyl,
N((C₁-C₄)alkyl)₂, or
NH—(C₃-C₆)cycloalkyl,
R₂ is H,
halo,
(C₁-C₈)alkyl,
(C₃-C₆)cycloalkyl,
O—(C₁-C₄)alkyl,
O—(C₃-C₆)cycloalkyl,
S—(C₁-C₄) alkyl,
S—(C₃-C₆)cycloalkyl,
NH₂,
NH(C₁-C₄)alkyl,
N((C₁-C₄)alkyl)₂, or
NH—(C₃-C₆)cycloalkyl;
R₃ and R₄ independently are halo,
(C₁-C₈)alkyl,
(C₃-C₆)cycloalkyl,
C—(C₁-C₄)alkyl,
C—(C₃-C₆)cycloalkyl,
S—(C₁-C₄) alkyl,
S—(C₃-C₆)cycloalkyl,
NH₂,
NH(C₁-C₄)alkyl,
N((C₁-C₄)alkyl)₂,
NH—(C₃-C₆)cycloalkyl;
aryl,
(CH₂)ₙ-aryl,
heterocyclo,
(CH₂)ₙ-heterocyclo,
heteroaryl, or
(CH₂)ₙ-heteroaryl,
wherein n is 0, 1, 2, or 3;
R₅ is H,
(C₁-C₈)alkyl,
(C₃-C₆)cycloalkyl,
aryl,
(CH₂)ₙ-aryl,
heterocyclo,
(CH₂)ₙ-heterocyclo,
heteroaryl, or
(CH₂)ₙ-heteroaryl,
wherein n is as defined above.

13. The compound of claim 12 as designated in formula IIA.

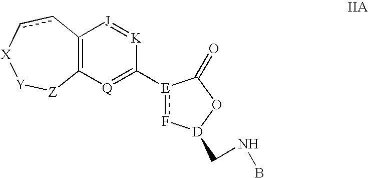

14. The compound of claim 12 as designated in formula IIB.

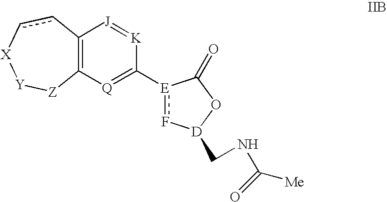

15. The compound of claim 12 as designated in formula IIC.

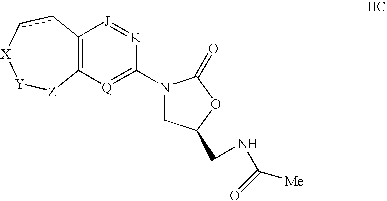

16. The compound of claim 12 as designated in formula IID. 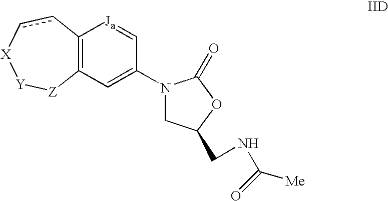
wherein Jₐ is N or CR₆, wherein R₆ is H or F.

17. The compound of claim 12 as designated in formula IIE.

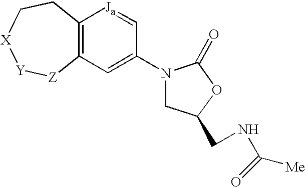

18. The compound of claim 17, wherein two of X, Y, or Z is C═C—R₅,
O═C,
NR₅,
N(C═O)R₅,
N(C═O)OR₅,
NSO₂R₅,
NSO₂NR₅,
O,
S,
SO, or
SO₂NR₅,
and the other of X, Y, or Z is CH₂ or CR₃R₄.

19. The compound of claim 18 as designated in formula IIF.

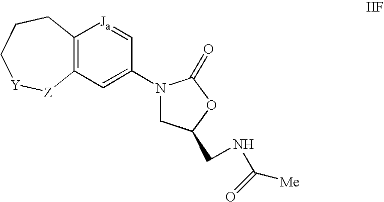

20. The compound of claim 18 as designated in formula IIG.

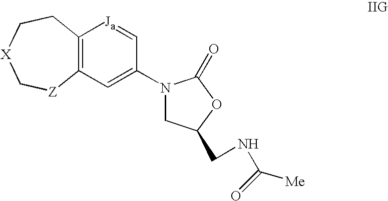

21. The compound of claim 20 as designated in formula IIH.

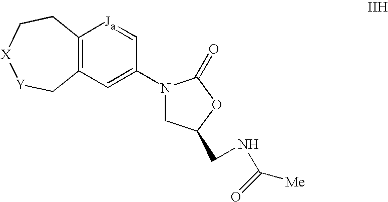

22. The compound of claim 20, wherein one of X, Y, or Z is C═C—R₅,
O═C,
NR₅,
N(C═O)R₅,
N(C═O)OR₅,
NSO₂R₅,
NSO₂NR₅,
O,
S,
SO, or
SO₂NR₅,
and the others of X, Y, or Z is CH₂.

23. A compound of formula III 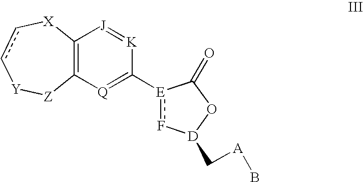
or a pharmaceutically acceptable salt thereof, wherein:
A is O,
NH, or
S;
B is C(═O)R₁,
C(═S)R₁,
heterocylco,
heteroaryl,
C(═O)-heterocyclo,
C(═N)—CN, or
C(═O)-heteteroaryl;
either D is N, E is C, and F is CH when “------” is a bond, or D is CH, E is N, and F is CH₂ when “------” is absent;
J, K, Q independently are CR₂ or N, with the proviso that when any one of J, K, or Q is N, then the other two are CR₂;
“------” is absent or is a bond;
X, Y, Z independently are C═C—R₅,
O═C,
CHR₃
CHR₄,
CR₃R₄,
NR₅,
N(C═O)R₅,
N(C═O)OR₅,
NSO₂R₅,
NSO₂NR₅,
O,
S,
SO, or
SO₂;
R₁ is H,
(C₁-C₈)alkyl,
(C₃-C₆)cycloalkyl,
O—(C₁-C₄)alkyl,
O—(C₃-C₆)cycloalkyl,
S—(C₁-C₄) alkyl,
S—(C₃-C₆)cycloalkyl,
NH₂,
NH(C₁-C₄)alkyl,
N((C₁-C₄)alkyl)₂, or
NH—(C₃-C₆)cycloalkyl;
R₂ is H,
halo,
(C₁-C₈)alkyl,
(C₃-C₆)cycloalkyl,
O—(C₁-C₄)alkyl,
O—(C₃-C₆)cycloalkyl,
S—(C₁-C₄) alkyl,
S—(C₃-C₆)cycloalkyl,
NH₂,
NH(C₁-C₄)alkyl,
N((C₁-C₄)alkyl)₂, or
NH—(C₃-C₆)cycloalkyl;
R₃ and R₄ independently are H,
halo,
(C₁-C₈)alkyl,
(C₃-C₆)cycloalkyl,
O—(C₁-C₄)alkyl,
O—(C₃-C₆)cycloalkyl,
S—(C₁-C₄)alkyl,
S—(C₃-C₆)cycloalkyl,
NH₂,
NH(C₁-C₄)alkyl,
N((C₁-C₄)alkyl)₂,
NH—(C₃-C₆)cycloalkyl;
aryl,
(CH₂)ₙ-aryl,
heterocyclo,
(CH₂)ₙ-heterocyclo,
heteroaryl, or
(CH₂)ₙ-heteroaryl,
wherein n is 0, 1, 2, or 3;
R₅ is H,
(C₁-C₈)alkyl,
(C₃-C₆)cycloalkyl,
aryl,
(CH₂)ₙ-aryl,
heterocyclo,
(CH₂)ₙ-heterocyclo,
heteroaryl, or
(CH₂)ₙ-heteroaryl,
wherein n is as defined above.

24. The compound of claim 23 as designated in formula IIIA.

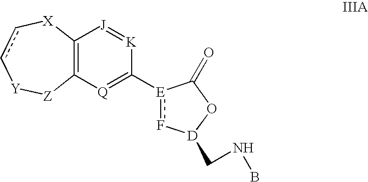

25. The compound of claim 23 as designated in formula IIIB.

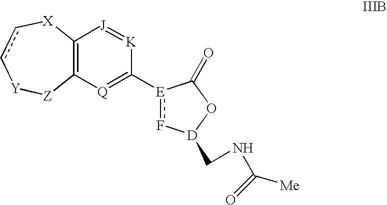

26. The compound of claim 23 as designated in formula IIIC.

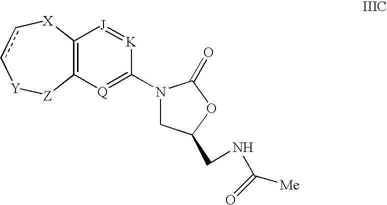

27. The compound of claim 27 as designated in formula IIID. 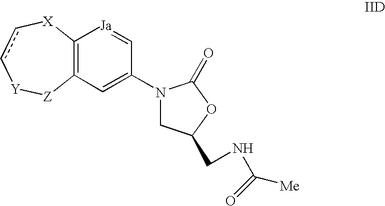
wherein Jₐ is N or CR₆, wherein R₆ is H or F.

28. The compound of claim 27 as designated in formula IIIE.

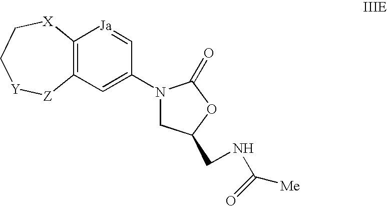

29. The compound of claim 28, wherein two of X, Y, or Z is C═C—R₅,
O═C,
NR₅,
N(C═O)R₅,
N(C═O)OR₅,
NSO₂R₅,
NSO₂NR₅,
O,
S,
SO, or
SO₂NR₅,
and the other of X, Y, or Z is CH₂ or CR₃R₄.

30. The compound of claim 29 as designated in formula IIIG.

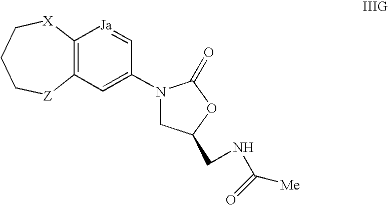

31. The compound of claim 29 as designated in formula IIIH.

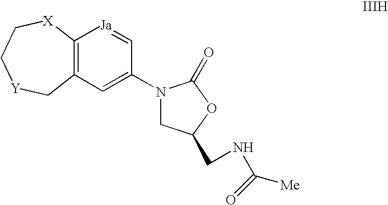

32. The compound of claim 28, wherein one of X, Y, or Z is C═C—R₅,
O═C,
NR₅,
N(C═O)R₅,
N(C═O)OR₅,
NSO₂R₅,
NSO₂NR₅,
O,
S,
SO, or
SO₂NR₅,
and the other of X, Y, or Z is CH₂.

33. A compound of formula IV: 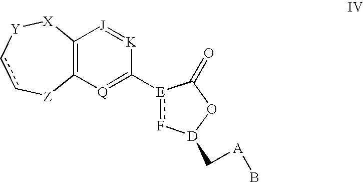
or a pharmaceutically acceptable salt thereof, wherein:
A is O,
NH, or
S;
B is
C(═O)R₁,
C(═S)R₁,
heterocylco,
heteroaryl,
C(═O)-heterocyclo,
C(═N)—CN, or
C(═O)-heteteroaryl;
either D is N, E is C, and F is CH when “------” is a bond, or D is CH, E is N, and F is CH₂ when “------” is absent;
J, K, Q independently are CR₂ or N, with the proviso that when any one of J, K, or Q is N, then the other two are CR₂;
“------” is absent; or is a bond; and
X, Y, Z independently are C═C—R₅,
O═C,
CH₂,
CHR₃,
CHR₄,
CR₃R₄,
NR₅,
N(C═O)R₅,
N(C═O)OR₅,
NSO₂R₅,
NSO₂NR₅,
O,
S,
SO, or
SO₂;
R₁ is H,
(C₁-C₈)alkyl,
(C₃-C₆)cycloalkyl,
O—(C₁-C₄)alkyl,
O—(C₃-C₆)cycloalkyl,
S—(C₁-C₄) alkyl,
S—(C₃-C₆)cycloalkyl,
NH₂,
NH(C₁-C₄)alkyl,
N((C₁-C₄)alkyl)₂, or
NH—(C₃-C₆)cycloalkyl,
R₂ is H,
halo,
(C₁-C₈)alkyl,
(C₃-C₆)cycloalkyl,
O—(C₁-C₄)alkyl,
O—(C₃-C₆)cycloalkyl,
S—(C₁-C₄) alkyl,
S—(C₃-C₆)cycloalkyl,
NH₂,
NH(C₁-C₄)alkyl,
N((C₁-C₄)alkyl)₂, or
NH—(C₃-C₆)cycloalkyl;
R₃ and R₄ independently are halo,
(C₁-C₈)alkyl,
(C₃-C₆)cycloalkyl,
O—(C₁-C₄)alkyl,
O—(C₃-C₆)cycloalkyl,
S—(C₁-C₄) alkyl,
S—(C₃-C₆)cycloalkyl,
NH₂,
NH(C₁-C₄)alkyl,
N((C₁-C₄)alkyl)₂,
NH—(C₃-C₆)cycloalkyl;
aryl,
(CH₂)ₙ-aryl,
heterocyclo,
(CH₂)ₙ-heterocyclo,
heteroaryl, or
(CH₂)ₙ-heteroaryl,
wherein n is 0, 1, 2, or 3;
R₅ is H,
(C₁-C₈)alkyl,
(C₃-C₆)cycloalkyl,
aryl,
(CH₂)ₙ-aryl,
heterocyclo,
(CH₂)ₙ-heterocyclo,
heteroaryl, or
(CH₂)ₙ-heteroaryl,
wherein n is as defined above.

34. The compound of claim 33 as designated in formula IVA.

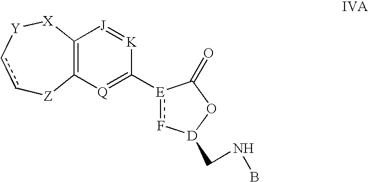

35. The compound of claim 33 as designated in formula IVB.

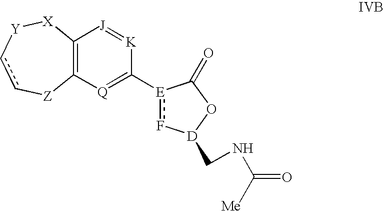

36. The compound of claim 33 as designated in formula IVC.

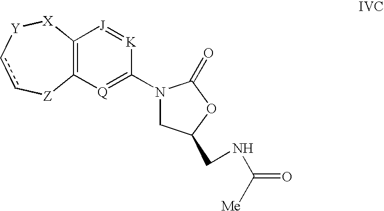

37. The compound of claim 33 as designated in formula IVD. 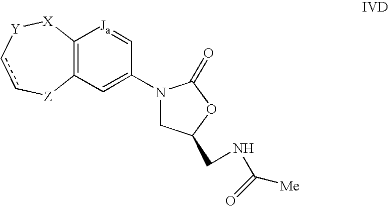
wherein Jₐ is N or CR₆, wherein R₆ is H or F.

38. The compound of claim 33 as designated in formula IVE.

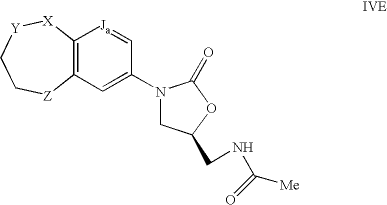

39. The compound of claim 38, wherein two of X, Y, or Z is C═C—R₅,
O═C,
NR₅,
N(C═O)R₅,
N(C═O)OR₅,
NSO₂R₅,
NSO₂NR₅,
O,
S,
SO, or
SO₂NR₅,
and the other of X, Y, or Z is CH₂ or CR₃R₄.

40. The compound of claim 39 as designated in formula IVF.

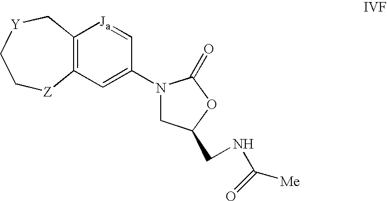

41. The compound of claim 39 as designated in formula IVG.

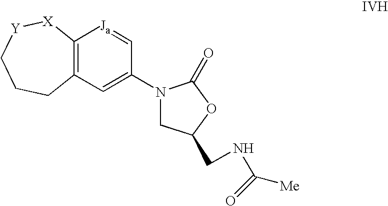

42. The compound of claim 38, wherein one of X, Y, or Z is C═C—R₅,
O═C,
NR₅,
N(C═O)R₅,
N(C═O)OR₅,
NSO₂R₅,
NSO₂NR₅,
O,
S,
SO, or
SO₂NR₅,
and the others of X, Y, or Z is CH₂.

43. A compound of formula V 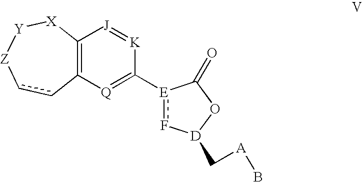
or a pharmaceutically acceptable salt thereof wherein:
A is O,
NH, or
S;
B is
C(═O)R₁,
C(═S)R₁,
heterocylco,
heteroaryl,
C(═O)-heterocyclo,
C(═N)—CN, or
C(═O)-heteteroaryl;
either D is N, E is C, and F is CH when “------” is a bond, or D is CH, E is N, and F is CH₂ when “------” is absent;
J, K, Q independently are CR₂ or N, with the proviso that when any one of J, K, or Q is N, then the other two are CR₂;
“------” is absent; or is a bond; and
X, Y, Z independently are C═C—R₅,
O═C,
CH₂,
CHR₃,
CHR₄,
CR₃R₄,
NR₅,
N(C═O)R₅,
N(C═O)OR₅,
NSO₂R₅,
NSO₂NR₅,
O,
S,
SO, or
SO₂;
R₁ is H,
(C₁-C₈)alkyl,
(C₃-C₆)cycloalkyl,
O—(C₁-C₄)alkyl,
O—(C₃-C₆)cycloalkyl,
S—(C₁-C₄) alkyl,
S—(C₃-C₆)cycloalkyl,
NH₂,
NH(C₁-C₄)alkyl,
N((C₁-C₄)alkyl)₂, or
NH—(C₃-C₆)cycloalkyl,
R₂ is H,
halo,
(C₁-C₈)alkyl,
(C₃-C₆)cycloalkyl,
O—(C₁-C₄)alkyl,
O—(C₃-C₆)cycloalkyl,
S—(C₁-C₄)alkyl,
S—(C₃-C₆)cycloalkyl,
NH₂,
NH(C₁-C₄)alkyl,
N((C₁-C₄)alkyl)₂, or
NH—(C₃-C₆)cycloalkyl;
R₃ and R₄ independently are halo,
(C₁-C₈)alkyl,
(C₃-C₆)cycloalkyl,
O—(C₁-C₄)alkyl,
O—(C₃-C₆)cycloalkyl,
S—(C₁-C₄) alkyl,
S—(C₃-C₆)cycloalkyl,
NH₂,
NH(C₁-C₄)alkyl,
N((C₁-C₄)alkyl)₂,
NH—(C₃-C₆)cycloalkyl;
aryl,
(CH₂)ₙ-aryl,
heterocyclo,
(CH₂)ₙ-heterocyclo,
heteroaryl, or
(CH₂)ₙ-heteroaryl,
wherein n is 0, 1, 2, or 3;
R₅ is H,
(C₁-C₈)alkyl,
(C₃-C₆)cycloalkyl,
aryl,
(CH₂)ₙ-aryl,
heterocyclo,
(CH₂)ₙ-heterocyclo,
heteroaryl, or
(CH₂)ₙ-heteroaryl,
wherein n is as defined above.

44. The compound of claim 43 as designated in formula VA.

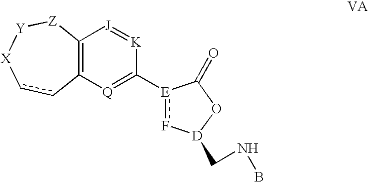

45. The compound of claim 43 as designated in formula VB.

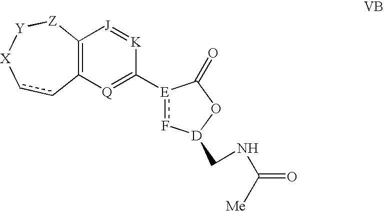

46. The compound of claim 43 as designated in formula VC.

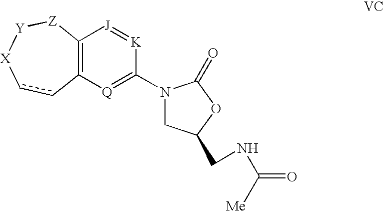

47. The compound of claim 43 as designated in formula VD 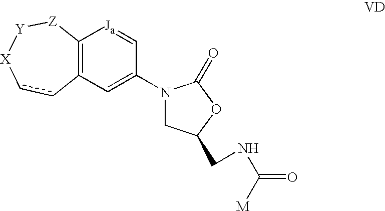
wherein Jₐ is N or CR₆, wherein R₆ is H or F.

48. The compound of claim 43 as designated in formula VE.

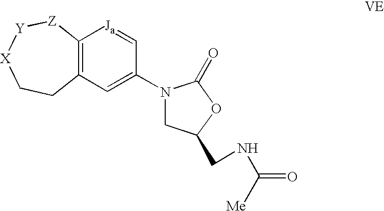

49. The compound of claim 48, wherein two of X, Y, or Z is C═C—R₅,
O═C,
NR₅,
N(C═O)R₅,
N(C═O)OR₅,
NSO₂R₅,
NSO₂NR₅,
O,
S,
SO, or
SO₂NR₅,
and the other of X, Y, or Z is CH₂ or CR₃R₄.

50. The compound of claim 51 as designated in formula VF.

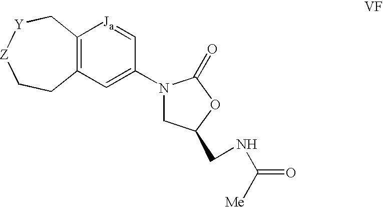

53. The compound of claim 51 as designated in formula VG.

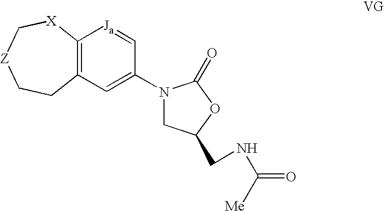

54. The compound of claim 48, wherein one of X, Y, or Z is C═C—R₅,
O═C,
NR₅,
N(C═O)R₅,
N(C═O)OR₅,
NSO₂R₅,
NSO₂NR₅,
O,
S,
SO, or
SO₂NR₅,
and the others of X, Y, or Z is CH₂.

55. A compound which is:
(S)-N-[2-Oxo-3-(6, 7,8,9-tetrahydro-5H-benzocyclohepten-2-yl)-oxazolidin-5-ylmethyl]-acetamide;
(S)-N-[2-Oxo-3-(5-oxo-6,7,8,9-tetrahydro-5H-benzocyclohepten-2-yl)-oxazolidin-5-ylmethyl]-acetamide;
(S)-N-[3-(6-Bromo-5-oxo-6,7,8,9-tetrahydro-5H-benzocyclohepten-2-yl)-2-oxo-oxazolidin-5-ylmethyl]-acetamide;
(S)-N-[3-(6-Dimethylaminomethylene-5-oxo-6,7,8,9-tetrahydro-5H-benzocyclohepten-2-yl)-2-oxo-oxazolidin-5-ylmethyl]-acetamide;
(S)-N-[2-Oxo-3-(5-oxo-6-pyridin-4-ylmethylene-6,7,8,9-tetrahydro-5H-benzocyclohepten-2-yl)-oxazolidin-5-ylmethyl]-acetamide;
(S)-N-[3-(6-Benzyl idene-5-oxo-6,7, 8,9-tetrahydro-5H-benzocyclohepten-2-yl)-2-oxo-oxazolidin-5-ylmethyl]-acetamide;
(S)-N-{3-[6-(4-Fluoro-benzylidene)-5-oxo-6,7,8,9-tetrahydro-5H-benzocyclohepten-2-yl]-2-oxo-oxazolidin-5-ylmethyl}-acetamide; (S)-N-[2-Oxo-3-(5-oxo-6-thiophen-3-ylmethylene-6,7,8,9-tetrahydro-5H-benzocyclohepten-2-yl)-oxazolidin-5-ylmethyl]-acetamide;
(S)-N-[3-(6-Furan-3-ylmethylene-5-oxo-6,7,8,9-tetrahydro-5H-benzocyclohepten-2-yl)-2-oxo-oxazolidin-5-ylmethyl]-acetamide;
(S)-N-[2-Oxo-3-(6-oxo-6,7,8,9-tetrahydro-5H-benzocyclohepten-2-yl)-oxazolidin-5-ylmethyl]-acetamide;
(S)-N-[2-Oxo-3-(7-oxo-6,7, 8,9-tetrahydro-5H-benzocyclohepten-2-yl)-oxazolidin-5-ylmethyl]-acetamide;
(S)-N-[2-Oxo-3-(8-oxo-6,7,8,9-tetrahydro-5H-benzocyclohepten-2-yl)-oxazolidin-5-ylmethyl]-acetamide;
(S)-N-[2-Oxo-3-(9-oxo-6,7, 8,9-tetrahydro-5H-benzocyclohepten-2-yl)-oxazolidin-5-ylmethyl]-acetamide;
(S)-N-[3-(8,9-Dihydro-7H-benzocyclohepten-2-yl)-2-oxo-oxazolidin-5-ylmethyl]-acetamide;
(S)-N-[3-(8,9-Dihydro-5H-benzocyclohepten-2-yl)-2-oxo-oxazol idin-5-ylmethyl]-acetamide;
(S)-N-[3-(6,9-Dihydro-5H-benzocyclohepten-2-yl)-2-oxo-oxazolidin-5-ylmethyl]-acetamide;
(S)-N-[3-(6,7-Dihydro-5H-benzocyclohepten-2-yl)-2-oxo-oxazol idin-5-ylmethyl]-acetamide; (S)-N-[2-Oxo-3-(2,3,4,5-tetrahydro-1H-benzo[b]azepin-7-yl)-oxazolidin-5-ylmethyl]-acetamide;
(S)-N-[2-Oxo-3-(2,3,4,5-tetrahydro-1H-benzo[c]azepin-7-yl)-oxazolidin-5-ylmethyl]-acetamide;
(S)-N-[2-Oxo-3-(2,3,4,5-tetrahydro-1H-benzo[d]azepin-7-yl)-oxazolidin-5-ylmethyl]-acetamide;
(S)-N-[2-Oxo-3-(2,3,4,5-tetrahydro-1H-benzo[c]azepin-8-yl)-oxazolidin-5-ylmethyl]-acetamide;
(S)-N-[2-Oxo-3-(2,3,4,5-tetrahydro-1H-benzo[b]azepin-8-yl)-oxazolidin-5-ylmethyl]-acetamide;
(S)-N-[2-Oxo-3-(2,3,4,5-tetrahydro-benzo[b]oxepin-7-yl)-oxazolidin-5-ylmethyl]-acetamide;
(S)-N-[2-Oxo-3-(1,3,4,5-tetrahydro-benzo[c]oxepin-7-yl)-oxazolidin-5-ylmethyl]-acetamide;
(S)-N-[2-Oxo-3-(5,6,8,9-tetrahydro-7-oxa-benzocyclohepten-2-yl)-oxazolidin-5-ylmethyl]-acetamide;
(S)-N-[2-Oxo-3-(1,3,4,5-tetrahydro-benzo[c]oxepin-8-yl)-oxazolidin-5-ylmethyl]-acetamide;
(S)-N-[2-Oxo-3-(2,3,4,5-tetrahydro-benzo[b]oxepin-8-yl)-oxazolidin-5-ylmethyl]-acetamide;
(S)-N-[2-Oxo-3-(6,7,8,9-tetrahydro-5-oxa-7-aza-benzocyclohepten-2-yl)-oxazolidin-5-ylmethyl]-acetamide;
(S)-N-[2-Oxo-3-(2,3,4,5-tetrahydro-benzo[b]thiepin-7-yl)-oxazolidin-5-ylmethyl]-acetamide;
(S)-N-[2-Oxo-3-(1,3,4,5-tetrahydro-benzo[c]thiepin-7-yl)-oxazol idin-5-ylmethyl]-acetamide;
(S)-N-[2-Oxo-3-(1,2,4,5-tetrahydro-benzo[d]thiepin-7-yl)-oxazolidin-5-ylmethyl]-acetamide;
(S)-N-[2-Oxo-3-(1,3,4,5-tetrahydro-benzo[c]thiepin-8-yl)-oxazolidin-5-ylmethyl]-acetamide;
(S)-N-[2-Oxo-3-(2,3,4,5-tetrahydro-benzo[b]thiepin-8-yl)-oxazol id in-5-ylmethyl]-acetamide;
(S)-N-[2-Oxo-3-(5-oxo-2,3,4, 5-tetrahydro-benzo[b]oxepin-8-yl)-oxazolidin-5-ylmethyl]-acetamide;
(S)-N-[2-Oxo-3-(5-oxo-2,3,4,5-tetrahydro-benzo[b]thiepin-8-yl)-oxazol idin-5-ylmethyl]-acetamide;
(S)-N-[2-Oxo-3-(1,1,5-trioxo-2,3,4,5-tetrahydro-1H-116-benzo[b]thiepin-8-yl)-oxazolidin-5-ylmethyl]-acetamide;
(S)-N-[2-Oxo-3-(5-oxo-2,3,4,5-tetrahydro-benzo[b]oxepin-7-yl)-oxazolidin-5-ylmethyl]-acetamide;
(S)-N-[3-(6,6-Difluoro-5-oxo-6,7, 8,9-tetrahydro-5H-benzocyclohepten-2-yl)-2-oxo-oxazolidin-5-ylmethyl]-acetamide;
(S)-N-[3-(6-Benzylidene-5-oxo-6,7,8,9-tetrahydro-5H-benzocyclohepten-2-yl)-2-oxo-oxazolidin-5-ylmethyl]-acetamide;
(S)-N-[3-(2-Methyl-2,3,4,5-tetrahydro-1H-benzo[c]azepin-7-yl)-2-oxo-oxazolidin-5-ylmethyl]-acetamide; or
(S)-N-[3-(3-Methyl-2,3,4,5-tetrahydro-1H-benzo[d]azepin-7-yl)-2-oxo-oxazolidin-5-ylmethyl]-acetamide.

56. A pharmaceutical formulation comprising a compound of claim 1 admixed with a pharmaceutically acceptable diluent, carrier, or excipient.

57. A method of treating a bacterial infection in a mammal, comprising administering to a mammal in need thereof an effective amount of a compound of claim 1.
